SEQUENCE LISTING (35 sequences)

SEQ ID NO: 1 - RNA, 30 nt, synthetic construct
  source 1..30: mol_type = other RNA
gtgctgtaggaagctcatctctcctatgtg

SEQ ID NO: 2 - RNA, 30 nt, synthetic construct
  source 1..30: mol_type = other RNA
ggtactcctggaagatgtccaccagggtct

SEQ ID NO: 3 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 4 - DNA, 250 bp, synthetic construct
  source 1..250: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 5 - RNA, 36 nt, synthetic construct
  source 1..36: mol_type = other RNA
gctgtgatagacctcgatttgtggggtagtaacagc

SEQ ID NO: 6 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 7 - skipped

SEQ ID NO: 8 - DNA, 54 bp, synthetic construct
  source 1..54: mol_type = other DNA
aagcgcacagccgacggcagcgagtttgaaagccctaagaagaaaagaaaggtg

SEQ ID NO: 9 - DNA, 2370 bp, synthetic construct
  source 1..2370: mol_type = other DNA
atgaacggaatcgagctgaagaaggaggaagccgccttttacttcaatcaggccgagctgaacttaaaggccatcgaggacaacatctttgacaaggagcggagaaagaccctgctgaataaccctcagatcctggccaagatggagaacttcatattcaacttcagagatgtgacaaagaacgccaagggcgagattgactgcctgctgctgaaactgagagaactgagaaacttctacagccactacgtgcacaagagagacgtgcgggaactatctaaaggcgagaagcctatcctggagaagtattaccagtttgccatcgaaagcaccggcagcgaaaacgtcaagctggaaatcatcgagaatgacgcttggctggctgatgccggggtgctgtttttcctgtgcatcttcctgaagaagtcccaagccaacaagctcattagcggcatcagcggcttcaagagaaacgacgctaccggccagccaagaagaaatctgttcacctacttcagcatcagagaaggctacaaggtggtgcccgagatgcagaagcacttcctgctgttctctctggtgaaccacctgagcaatcaggacgactatatcgagaaggcccaccaaccctacgacatcggagagggcctgttcttccacagaatcgccagcaccttcctgaacatcagcggcatcctgagaaacatgaaattctacacataccaaagcaaaagactggtcgagcagagaggcgagctgaaaagggagaaggacattttcgcctgggaagagcctttccagggcaacagctacttcgagatcaatggccacaaaggagttatcggcgaagatgaactgaaggaactgtgttacgccttcctgatcggcaatcaagacgccaacaaggtagaaggcagaatcacacagttcctggaaaagttcagaaacgccaacagcgtgcagcaggtgaaggacgatgaaatgctgaagcctgaatactttcctgcaaactactttgccgagagcggtgtgggcagaatcaaggaccgggtgctgaaccggctgaacaaggccataaagagcaataaagctaagaagggcgaaatcatcgcctacgataagatgcgggaagtgatggccttcatcaacaacagcctaccagtggatgagaagctgaagcccaaggattacaagcggtacctgggaatggtccggttctgggatagagagaaggataacatcaagcgcgaattcgagacaaaagagtggtccaaatacctgcctagcaatttctggaccgccaagaacctggagcgggtgtacggactggcccgtgagaaaaacgcagagctgttcaacaagctgaaagctgacgtggaaaaaatggatgagagagaactggagaaataccagaagatcaacgacgccaaagacctggctaacctgcggcggctggcctctgacttcggcgtgaagtgggaagagaaggactgggacgagtactctggccagatcaagaagcagatcaccgacagccagaagctgaccatcatgaagcagagaattaccgcaggcctgaaaaaaaagcacggcatcgaaaacctgaacctgagaatcaccatcgacatcaataagagcagaaaagccgtgctgaatagaattgccatccctagaggctttgtgaagagacatatcctcggatggcaggagtctgagaaggtgtccaaaaaaatcagagaagccgagtgcgagatcctgctctccaaggagtatgaagagctgagcaaacagttcttccagagcaaggactacgacaaaatgaccagaatcaacggcctgtacgagaaaaacaagcttatcgccctgatggccgtgtacctgatgggccagctgcggatcctgtttaaggaacacacaaagctggctgatatcacaaaaaccacagtggatttcaagatcagcgacaaggtgacagtgaagattcccttcagcaacgctcctagcctggtgtataccatgagcagcaaggccgtggacaacatcggaaattacggcttcagcaacaaagacaaggataagcccatcctgggcaagatcgatgtgatcgagaagcagcggatggagtttatcaaggaagtcctgggattcgagaagtacctgttcgatgataagatcatcgacaagagcaagttcgccgacaccgccacccacatctctttcgccgagatcgttgaagagctggtggaaaagggatgggacaaagatcggctgaccaagctgaaggacgctagaaacaaggctctgcatggcgaaatcctgaccggcaccagcttcgacgagacaaagtccctgatcaacgagctgaagaag

SEQ ID NO: 10 - DNA, 54 bp, synthetic construct
  source 1..54: mol_type = other DNA
aagagaaccgccgacggctctgagttcgaaagtcctaagaagaagcggaaggtg

SEQ ID NO: 11 - DNA, 248 bp, synthetic construct
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 12 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 13 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 4516 bp, synthetic construct
  source 1..4516: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccatcgatactagtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctgtgatagacctcgatttgtggggtagtaacagcgtgctgtaggaagctcatctctcctatgtggctgtgatagacctcgatttgtggggtagtaacagcggtactcctggaagatgtccaccagggtctgctgtgatagacctcgatttgtggggtagtaacagcttttttttacgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgaagcgcacagccgacggcagcgagtttgaaagccctaagaagaaaagaaaggtgaacggaatcgagctgaagaaggaggaagccgccttttacttcaatcaggccgagctgaacttaaaggccatcgaggacaacatctttgacaaggagcggagaaagaccctgctgaataaccctcagatcctggccaagatggagaacttcatattcaacttcagagatgtgacaaagaacgccaagggcgagattgactgcctgctgctgaaactgagagaactgagaaacttctacagccactacgtgcacaagagagacgtgcgggaactatctaaaggcgagaagcctatcctggagaagtattaccagtttgccatcgaaagcaccggcagcgaaaacgtcaagctggaaatcatcgagaatgacgcttggctggctgatgccggggtgctgtttttcctgtgcatcttcctgaagaagtcccaagccaacaagctcattagcggcatcagcggcttcaagagaaacgacgctaccggccagccaagaagaaatctgttcacctacttcagcatcagagaaggctacaaggtggtgcccgagatgcagaagcacttcctgctgttctctctggtgaaccacctgagcaatcaggacgactatatcgagaaggcccaccaaccctacgacatcggagagggcctgttcttccacagaatcgccagcaccttcctgaacatcagcggcatcctgagaaacatgaaattctacacataccaaagcaaaagactggtcgagcagagaggcgagctgaaaagggagaaggacattttcgcctgggaagagcctttccagggcaacagctacttcgagatcaatggccacaaaggagttatcggcgaagatgaactgaaggaactgtgttacgccttcctgatcggcaatcaagacgccaacaaggtagaaggcagaatcacacagttcctggaaaagttcagaaacgccaacagcgtgcagcaggtgaaggacgatgaaatgctgaagcctgaatactttcctgcaaactactttgccgagagcggtgtgggcagaatcaaggaccgggtgctgaaccggctgaacaaggccataaagagcaataaagctaagaagggcgaaatcatcgcctacgataagatgcgggaagtgatggccttcatcaacaacagcctaccagtggatgagaagctgaagcccaaggattacaagcggtacctgggaatggtccggttctgggatagagagaaggataacatcaagcgcgaattcgagacaaaagagtggtccaaatacctgcctagcaatttctggaccgccaagaacctggagcgggtgtacggactggcccgtgagaaaaacgcagagctgttcaacaagctgaaagctgacgtggaaaaaatggatgagagagaactggagaaataccagaagatcaacgacgccaaagacctggctaacctgcggcggctggcctctgacttcggcgtgaagtgggaagagaaggactgggacgagtactctggccagatcaagaagcagatcaccgacagccagaagctgaccatcatgaagcagagaattaccgcaggcctgaaaaaaaagcacggcatcgaaaacctgaacctgagaatcaccatcgacatcaataagagcagaaaagccgtgctgaatagaattgccatccctagaggctttgtgaagagacatatcctcggatggcaggagtctgagaaggtgtccaaaaaaatcagagaagccgagtgcgagatcctgctctccaaggagtatgaagagctgagcaaacagttcttccagagcaaggactacgacaaaatgaccagaatcaacggcctgtacgagaaaaacaagcttatcgccctgatggccgtgtacctgatgggccagctgcggatcctgtttaaggaacacacaaagctggctgatatcacaaaaaccacagtggatttcaagatcagcgacaaggtgacagtgaagattcccttcagcaacgctcctagcctggtgtataccatgagcagcaaggccgtggacaacatcggaaattacggcttcagcaacaaagacaaggataagcccatcctgggcaagatcgatgtgatcgagaagcagcggatggagtttatcaaggaagtcctgggattcgagaagtacctgttcgatgataagatcatcgacaagagcaagttcgccgacaccgccacccacatctctttcgccgagatcgttgaagagctggtggaaaagggatgggacaaagatcggctgaccaagctgaaggacgctagaaacaaggctctgcatggcgaaatcctgaccggcaccagcttcgacgagacaaagtccctgatcaacgagctgaagaagtccggcggctctaagagaaccgccgacggctctgagttcgaaagtcctaagaagaagcggaaggtgtgagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - protein, 18 aa, synthetic construct
KRTADGSEFESPKKKRKV

SEQ ID NO: 16 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDATGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLADITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 17 - RNA, 168 nt, synthetic construct
  source 1..168: mol_type = other RNA
gctgtgatagacctcgatttgtggggtagtaacagcgtgctgtaggaagctcatctctcctatgtggctgtgatagacctcgatttgtggggtagtaacagcggtactcctggaagatgtccaccagggtctgctgtgatagacctcgatttgtggggtagtaacagc

SEQ ID NO: 18 - DNA, 261 bp, synthetic construct
  source 1..261: mol_type = other DNA
taggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggttgg

SEQ ID NO: 19 - DNA, 21 bp, synthetic construct
  source 1..21: mol_type = other DNA
cccaagaagaagcggaaggtg

SEQ ID NO: 20 - DNA, 2325 bp, synthetic construct
  source 1..2325: mol_type = other DNA
atggcccaggtgagcaagcagacctccaagaagagggagctgagcatcgacgagtaccagggcgcccggaagtggtgcttcaccattgccttcaacaaggccctggtgaaccgggacaagaacgacggcctgttcgtggaaagcctgctgagacacgagaagtacagcaagcacgactggtacgacgaagatacccgggccctgatcaagtgcagcacccaggccgccaacgccaaggctgaagccctgcggaactacttcagtcactaccggcatagccctggctgcctgaccttcaccgccgaggacgaactgcggaccatcatggagagagcctatgagcgggccatcttcgagtgcagaagaagagagacagaggtgatcatcgagtttcccagcctgttcgagggcgaccggatcaccaccgccggcgtggtgtttttcgtgagctttttcgtggaaagaagagtgctggatcggctgtatggagccgtgtccggcctgaagaagaatgagggacagtacaagctgacccggaaggccctgagcatgtactgcctgaaggacagcagattcaccaaggcctgggataagcgggtgctgctgttcagagacatcctggcccagctgggaagaatccccgccgaggcctacgagtactaccacggcgagcagggtgataagaagagagctaacgacaatgagggcacaaatcccaagcggcacaaggacaagttcatcgaatttgcactgcactacctggaagcccagcacagcgagatctgcttcggcagacgccacatcgtgcgggaagaggccggcgccggcgatgagcacaagaagcaccggaccaagggaaaggtggtggtggacttcagcaagaaggacgaggaccagagctactatatctccaagaacaacgtgatcgtgcggatcgacaagaacgccggccctagaagctaccggatgggcctgaacgagctgaagtacctcgtgctgctgagcctgcaggggaagggcgacgatgccatcgccaagctgtacagatacagacagcacgtggagaacatcctggatgtggtgaaggtgaccgataaggataaccacgtgttcctgccccgcttcgtgctggagcagcacggcatcggcagaaaggccttcaagcagcggatcgatggacgggtgaagcacgtgcggggcgtgtgggagaagaagaaggccgccaccaatgaaatgaccctgcacgagaaggccagagacatcctgcagtacgtgaacgaaaactgcacccggtccttcaaccctggcgaatacaacagactgctggtgtgcctggtgggcaaggacgtggagaactttcaggccggcctgaagcggctgcagctggccgaaaggatcgatggccgggtgtactccatcttcgcccagaccagcaccatcaatgagatgcaccaggtggtgtgcgaccagatcctgaaccggctgtgcagaatcggcgaccagaagctgtacgattacgtgggactgggcaagaaggacgaaatcgactacaagcagaaggtggcctggttcaaggagcacatcagcatccggagaggattcctgagaaagaagttctggtacgatagcaagaagggattcgcaaagctggtggaggaacacctggagtccggcggcggccagcgcgacgtgggcctggacaagaagtactaccacatcgacgccatcggcagattcgagggcgccaaccccgccctgtacgagaccctggccagagatcggctgtgcctcatgatggcccagtacttcctgggcagcgtgagaaaggaactgggcaacaagattgtgtggagcaacgacagcatcgaactgcctgtggaaggctctgtgggaaatgagaagagcatcgtgttctccgtgtctgactacggcaagctgtacgtgctggacgatgccgaattcctgggccggatctgcgaatacttcatgccccacgaaaagggcaagatccgggcccacacagtggccgaaaagggctttagagcatacaacgacctgcagaagaagtgcgtggaggccgtgctggctttcgaagagaaggtggtgaaggccaagaagatgagcgagaaggaaggcgcccactacatcgacttccgggagatcctggcccagaccatgtgcaaggaggccgaaaagaccgcagtgaacaaggtgagacgcgccttcttccaccaccacctgaagttcgtgattgacgagttcggcctgttcagcgacgtgatgaagaagtacggcatcgagaaggaatggaagttccctgtcaag

SEQ ID NO: 21 - DNA, 135 bp, synthetic construct
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 22 - DNA, 250 bp, synthetic construct
  source 1..250: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 23 - RNA, 36 nt, synthetic construct
  source 1..36: mol_type = other RNA
gctggagcagcccccgatttgtggggtgattacagc

SEQ ID NO: 24 - DNA, 3569 bp, synthetic construct
  source 1..3569: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccatcgatggcgcgccggattcgacattgattattgataggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggttggaccggtgccaccatgcccaagaagaagcggaaggtggcccaggtgagcaagcagacctccaagaagagggagctgagcatcgacgagtaccagggcgcccggaagtggtgcttcaccattgccttcaacaaggccctggtgaaccgggacaagaacgacggcctgttcgtggaaagcctgctgagacacgagaagtacagcaagcacgactggtacgacgaagatacccgggccctgatcaagtgcagcacccaggccgccaacgccaaggctgaagccctgcggaactacttcagtcactaccggcatagccctggctgcctgaccttcaccgccgaggacgaactgcggaccatcatggagagagcctatgagcgggccatcttcgagtgcagaagaagagagacagaggtgatcatcgagtttcccagcctgttcgagggcgaccggatcaccaccgccggcgtggtgtttttcgtgagctttttcgtggaaagaagagtgctggatcggctgtatggagccgtgtccggcctgaagaagaatgagggacagtacaagctgacccggaaggccctgagcatgtactgcctgaaggacagcagattcaccaaggcctgggataagcgggtgctgctgttcagagacatcctggcccagctgggaagaatccccgccgaggcctacgagtactaccacggcgagcagggtgataagaagagagctaacgacaatgagggcacaaatcccaagcggcacaaggacaagttcatcgaatttgcactgcactacctggaagcccagcacagcgagatctgcttcggcagacgccacatcgtgcgggaagaggccggcgccggcgatgagcacaagaagcaccggaccaagggaaaggtggtggtggacttcagcaagaaggacgaggaccagagctactatatctccaagaacaacgtgatcgtgcggatcgacaagaacgccggccctagaagctaccggatgggcctgaacgagctgaagtacctcgtgctgctgagcctgcaggggaagggcgacgatgccatcgccaagctgtacagatacagacagcacgtggagaacatcctggatgtggtgaaggtgaccgataaggataaccacgtgttcctgccccgcttcgtgctggagcagcacggcatcggcagaaaggccttcaagcagcggatcgatggacgggtgaagcacgtgcggggcgtgtgggagaagaagaaggccgccaccaatgaaatgaccctgcacgagaaggccagagacatcctgcagtacgtgaacgaaaactgcacccggtccttcaaccctggcgaatacaacagactgctggtgtgcctggtgggcaaggacgtggagaactttcaggccggcctgaagcggctgcagctggccgaaaggatcgatggccgggtgtactccatcttcgcccagaccagcaccatcaatgagatgcaccaggtggtgtgcgaccagatcctgaaccggctgtgcagaatcggcgaccagaagctgtacgattacgtgggactgggcaagaaggacgaaatcgactacaagcagaaggtggcctggttcaaggagcacatcagcatccggagaggattcctgagaaagaagttctggtacgatagcaagaagggattcgcaaagctggtggaggaacacctggagtccggcggcggccagcgcgacgtgggcctggacaagaagtactaccacatcgacgccatcggcagattcgagggcgccaaccccgccctgtacgagaccctggccagagatcggctgtgcctcatgatggcccagtacttcctgggcagcgtgagaaaggaactgggcaacaagattgtgtggagcaacgacagcatcgaactgcctgtggaaggctctgtgggaaatgagaagagcatcgtgttctccgtgtctgactacggcaagctgtacgtgctggacgatgccgaattcctgggccggatctgcgaatacttcatgccccacgaaaagggcaagatccgggcccacacagtggccgaaaagggctttagagcatacaacgacctgcagaagaagtgcgtggaggccgtgctggctttcgaagagaaggtggtgaaggccaagaagatgagcgagaaggaaggcgcccactacatcgacttccgggagatcctggcccagaccatgtgcaaggaggccgaaaagaccgcagtgaacaaggtgagacgcgccttcttccaccaccacctgaagttcgtgattgacgagttcggcctgttcagcgacgtgatgaagaagtacggcatcgagaaggaatggaagttccctgtcaagcccaagaagaagcggaaggtgtagaagcttaacttgtttattgcagcttataatggttacaaataaagcaatagcatcacaaatttcacaaataaagcatttttttcactgcattctagttgtggtttgtccaaactcatcaatgtatcttatcatgtctggatcactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctggagcagcccccgatttgtggggtgattacagcgtgctgtaggaagctcatctctcctatgtggctggagcagcccccgatttgtggggtgattacagcggtactcctggaagatgtccaccagggtctgctggagcagcccccgatttgtggggtgattacagctttttttacgcgttagttattaatagtgcgtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - RNA, 168 nt, synthetic construct
  source 1..168: mol_type = other RNA
gctggagcagcccccgatttgtggggtgattacagcgtgctgtaggaagctcatctctcctatgtggctggagcagcccccgatttgtggggtgattacagcggtactcctggaagatgtccaccagggtctgctggagcagcccccgatttgtggggtgattacagc

SEQ ID NO: 26 - protein, 7 aa, synthetic construct
PKKKRKV

SEQ ID NO: 27 - protein, 775 aa, synthetic construct
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRAHTVAEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 28 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 29 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - RNA, 102 nt, synthetic construct
  source 1..102: mol_type = other RNA
gctgtgatagacctcgatttgtggggtagtaacagcgtgctgtaggaagctcatctctcctatgtggctgtgatagacctcgatttgtggggtagtaacagc

SEQ ID NO: 31 - RNA, 102 nt, synthetic construct
  source 1..102: mol_type = other RNA
gctgtgatagacctcgatttgtggggtagtaacagcggtactcctggaagatgtccaccagggtctgctgtgatagacctcgatttgtggggtagtaacagc

SEQ ID NO: 32 - protein, 789 aa, synthetic construct
NGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDATGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLADITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 33 - DNA, 2367 bp, synthetic construct
  source 1..2367: mol_type = other DNA
aacggaatcgagctgaagaaggaggaagccgccttttacttcaatcaggccgagctgaacttaaaggccatcgaggacaacatctttgacaaggagcggagaaagaccctgctgaataaccctcagatcctggccaagatggagaacttcatattcaacttcagagatgtgacaaagaacgccaagggcgagattgactgcctgctgctgaaactgagagaactgagaaacttctacagccactacgtgcacaagagagacgtgcgggaactatctaaaggcgagaagcctatcctggagaagtattaccagtttgccatcgaaagcaccggcagcgaaaacgtcaagctggaaatcatcgagaatgacgcttggctggctgatgccggggtgctgtttttcctgtgcatcttcctgaagaagtcccaagccaacaagctcattagcggcatcagcggcttcaagagaaacgacgctaccggccagccaagaagaaatctgttcacctacttcagcatcagagaaggctacaaggtggtgcccgagatgcagaagcacttcctgctgttctctctggtgaaccacctgagcaatcaggacgactatatcgagaaggcccaccaaccctacgacatcggagagggcctgttcttccacagaatcgccagcaccttcctgaacatcagcggcatcctgagaaacatgaaattctacacataccaaagcaaaagactggtcgagcagagaggcgagctgaaaagggagaaggacattttcgcctgggaagagcctttccagggcaacagctacttcgagatcaatggccacaaaggagttatcggcgaagatgaactgaaggaactgtgttacgccttcctgatcggcaatcaagacgccaacaaggtagaaggcagaatcacacagttcctggaaaagttcagaaacgccaacagcgtgcagcaggtgaaggacgatgaaatgctgaagcctgaatactttcctgcaaactactttgccgagagcggtgtgggcagaatcaaggaccgggtgctgaaccggctgaacaaggccataaagagcaataaagctaagaagggcgaaatcatcgcctacgataagatgcgggaagtgatggccttcatcaacaacagcctaccagtggatgagaagctgaagcccaaggattacaagcggtacctgggaatggtccggttctgggatagagagaaggataacatcaagcgcgaattcgagacaaaagagtggtccaaatacctgcctagcaatttctggaccgccaagaacctggagcgggtgtacggactggcccgtgagaaaaacgcagagctgttcaacaagctgaaagctgacgtggaaaaaatggatgagagagaactggagaaataccagaagatcaacgacgccaaagacctggctaacctgcggcggctggcctctgacttcggcgtgaagtgggaagagaaggactgggacgagtactctggccagatcaagaagcagatcaccgacagccagaagctgaccatcatgaagcagagaattaccgcaggcctgaaaaaaaagcacggcatcgaaaacctgaacctgagaatcaccatcgacatcaataagagcagaaaagccgtgctgaatagaattgccatccctagaggctttgtgaagagacatatcctcggatggcaggagtctgagaaggtgtccaaaaaaatcagagaagccgagtgcgagatcctgctctccaaggagtatgaagagctgagcaaacagttcttccagagcaaggactacgacaaaatgaccagaatcaacggcctgtacgagaaaaacaagcttatcgccctgatggccgtgtacctgatgggccagctgcggatcctgtttaaggaacacacaaagctggctgatatcacaaaaaccacagtggatttcaagatcagcgacaaggtgacagtgaagattcccttcagcaacgctcctagcctggtgtataccatgagcagcaaggccgtggacaacatcggaaattacggcttcagcaacaaagacaaggataagcccatcctgggcaagatcgatgtgatcgagaagcagcggatggagtttatcaaggaagtcctgggattcgagaagtacctgttcgatgataagatcatcgacaagagcaagttcgccgacaccgccacccacatctctttcgccgagatcgttgaagagctggtggaaaagggatgggacaaagatcggctgaccaagctgaaggacgctagaaacaaggctctgcatggcgaaatcctgaccggcaccagcttcgacgagacaaagtccctgatcaacgagctgaagaag

SEQ ID NO: 34 - protein, 774 aa, synthetic construct
AQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRAHTVAEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 35 - DNA, 2322 bp, synthetic construct
  source 1..2322: mol_type = other DNA
gcccaggtgagcaagcagacctccaagaagagggagctgagcatcgacgagtaccagggcgcccggaagtggtgcttcaccattgccttcaacaaggccctggtgaaccgggacaagaacgacggcctgttcgtggaaagcctgctgagacacgagaagtacagcaagcacgactggtacgacgaagatacccgggccctgatcaagtgcagcacccaggccgccaacgccaaggctgaagccctgcggaactacttcagtcactaccggcatagccctggctgcctgaccttcaccgccgaggacgaactgcggaccatcatggagagagcctatgagcgggccatcttcgagtgcagaagaagagagacagaggtgatcatcgagtttcccagcctgttcgagggcgaccggatcaccaccgccggcgtggtgtttttcgtgagctttttcgtggaaagaagagtgctggatcggctgtatggagccgtgtccggcctgaagaagaatgagggacagtacaagctgacccggaaggccctgagcatgtactgcctgaaggacagcagattcaccaaggcctgggataagcgggtgctgctgttcagagacatcctggcccagctgggaagaatccccgccgaggcctacgagtactaccacggcgagcagggtgataagaagagagctaacgacaatgagggcacaaatcccaagcggcacaaggacaagttcatcgaatttgcactgcactacctggaagcccagcacagcgagatctgcttcggcagacgccacatcgtgcgggaagaggccggcgccggcgatgagcacaagaagcaccggaccaagggaaaggtggtggtggacttcagcaagaaggacgaggaccagagctactatatctccaagaacaacgtgatcgtgcggatcgacaagaacgccggccctagaagctaccggatgggcctgaacgagctgaagtacctcgtgctgctgagcctgcaggggaagggcgacgatgccatcgccaagctgtacagatacagacagcacgtggagaacatcctggatgtggtgaaggtgaccgataaggataaccacgtgttcctgccccgcttcgtgctggagcagcacggcatcggcagaaaggccttcaagcagcggatcgatggacgggtgaagcacgtgcggggcgtgtgggagaagaagaaggccgccaccaatgaaatgaccctgcacgagaaggccagagacatcctgcagtacgtgaacgaaaactgcacccggtccttcaaccctggcgaatacaacagactgctggtgtgcctggtgggcaaggacgtggagaactttcaggccggcctgaagcggctgcagctggccgaaaggatcgatggccgggtgtactccatcttcgcccagaccagcaccatcaatgagatgcaccaggtggtgtgcgaccagatcctgaaccggctgtgcagaatcggcgaccagaagctgtacgattacgtgggactgggcaagaaggacgaaatcgactacaagcagaaggtggcctggttcaaggagcacatcagcatccggagaggattcctgagaaagaagttctggtacgatagcaagaagggattcgcaaagctggtggaggaacacctggagtccggcggcggccagcgcgacgtgggcctggacaagaagtactaccacatcgacgccatcggcagattcgagggcgccaaccccgccctgtacgagaccctggccagagatcggctgtgcctcatgatggcccagtacttcctgggcagcgtgagaaaggaactgggcaacaagattgtgtggagcaacgacagcatcgaactgcctgtggaaggctctgtgggaaatgagaagagcatcgtgttctccgtgtctgactacggcaagctgtacgtgctggacgatgccgaattcctgggccggatctgcgaatacttcatgccccacgaaaagggcaagatccgggcccacacagtggccgaaaagggctttagagcatacaacgacctgcagaagaagtgcgtggaggccgtgctggctttcgaagagaaggtggtgaaggccaagaagatgagcgagaaggaaggcgcccactacatcgacttccgggagatcctggcccagaccatgtgcaaggaggccgaaaagaccgcagtgaacaaggtgagacgcgccttcttccaccaccacctgaagttcgtgattgacgagttcggcctgttcagcgacgtgatgaagaagtacggcatcgagaaggaatggaagttccctgtcaag

(19) United States
(12) Patent Application Publication
(10) Pub. No.: US 2026/0286348 A1
(43) Pub. Date: Sep. 24, 2026

(54) GUIDE NUCLEIC ACIDS TARGETING VEGFA AND USES THEREOF

(71) Applicant: HuidaGene Therapeutics (Singapore) Pte. Ltd., Singapore (SG)

(72) Inventor: Xing Wang, Shanghai (CN)

(21) Appl. No.: 19/474,214

(22) PCT Filed: Apr. 12, 2024
(86) PCT No.: PCT/CN2024/087570

§ 371 Date: Oct. 10, 2025

(30) Foreign Application Priority Data
Apr. 12, 2023 (WO) PCT/CN2023/087915
Apr. 21, 2023 (WO) PCT/CN2023/089908

(51) Int. Cl. C12N 15/11; C12N 9/22; C12N 15/86

(52) U.S. Cl. CPC C12N 15/11; C12N 9/226; C12N 15/86; C12N 2310/20; C12N 2750/14143; C12N 2750/14151; C12N 2830/48; C12N 2830/50

(57) ABSTRACT

Provided are systems and methods for modifying VEGFA expression.

### REFERENCE TO RELATED APPLICATIONS

The instant application claims the priority to and the benefit of the filing date (is related to) of PCT/CN2023/087915, filed on Apr. 12, 2023, and PCT/CN2023/089908, filed on Apr. 21, 2023, the entire contents of which, including any drawings and sequence listing, are incorporated herein by reference.

### REFERENCE TO AN ELECTRONIC SEQUENCE LISTING

The disclosure contains a Sequence Listing XML file which has been submitted electronically in XML format and is hereby incorporated by reference in its entirety. Said XML copy, created on Apr. 12, 2024, by software “WIPO Sequence” according to WIPO Standard ST.26, is named HGP024PCT.xml, and is 54,485 bytes in size.

According to WIPO Standard ST.26, symbol “t” is used to denote both T in DNA and U in RNA. Thus, in the instant sequence listing prepared according to ST.26, wherever a sequence is an RNA, the T in the sequence shall be deemed as U.

### BACKGROUND

VEGFA gene (also known as VPF, VEGF, or MVCD1) encodes vascular endothelial growth factor A (VEGFA), which plays a vital role in both physiological development of vascular structures and pathological angiogenesis. It has been reported that VEGFA is associated with diseases, such as, neovascular age-related macular degeneration (nAMD) (also known as wet AMD) and Diabetic Macular Edema (DME). AMD is a major cause of visual dysfunction for adults over age 50 of which nAMD accounted for 90% blindness in AMD. Choroidal neovascularization (CNV) is the hallmark of nAMD. Overexpression of VEGFA promotes CNV. WO2022/188797 (PCT/CN2022/079890), incorporated herein by reference in its entirety, discloses knockdown of VEGFA mRNA by AAV9-delivered hfCas13X.1 (also known as hfCas13e.1) system. It would be desired to develop tools and methods to knockdown VEGFA mRNA and/or treat VEGFA associated diseases with improved efficiency and/or safety.

Citation or identification of any document in the disclosure is not an admission that such a document is available as prior art to the disclosure. Each of the references mentioned or cited in the disclosure is incorporated by reference in its entirety.

### SUMMARY

The disclosure provides, in some aspects, tools and methods to downregulate VEGFA expression with improved efficiency and/or safety. FIG. 12 illustrates a representative high-fidelity Cas13Y (hfCas13Y) endonuclease based therapeutic system provided in some aspects of the disclosure. FIG. 12A shows schematic of the representative therapeutic system HG202 based on the high fidelity Cas13Y endonuclease developed by the Applicant for specific and efficient knockdown of a target RNA. FIG. 12B illustrates the mechanism of the therapeutic system HG202, where HG202 downregulates the expression of VEGFA by hfCas13Y based VEGFA mRNA knockdown with improved efficiency and/or safety.

In an aspect, the disclosure provides a system comprising:
(1) a guide nucleic acid comprising
   a) a scaffold sequence capable of forming a complex with a Cas13 endonuclease, and
   b) a guide sequence capable of hybridizing to a target sequence on a transcript of a VEGFA gene, thereby guiding the complex to the transcript, or a polynucleotide encoding the guide nucleic acid; and
(2) the Cas13 endonuclease or a polynucleotide encoding the Cas13 endonuclease;
   wherein the Cas13 endonuclease comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 16 or 32, comprises substitutions D160A, D642A, Y666A, and Y677A relative to the wild type of the Cas13 endonuclease of SEQ ID NO: 16, and having substantially the same (e.g., at least about 80%, 90%, 95%, 99%, or more) guide sequence-specific RNA endonuclease activity as the Cas13 endonuclease of SEQ ID NO: 16;
   wherein the guide sequence comprises (1) a sequence of SEQ ID NO: 1 or 2 or a 5′ or 3′ end truncation thereof with 1, 2, 3, 4, 5, or 6 nucleotides truncated at the 5′ or 3′ end; or (2) a sequence having a sequence identity of at least about 70%, 75%, 80%, 85%, 90%, 95%, or 100% to SEQ ID NO: 1 or 2 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 1 or 2.

In some embodiments, the scaffold sequence has substantially the same secondary structure as the secondary structure of the sequence of SEQ ID NO: 5; or wherein the scaffold sequence comprises (1) a sequence of SEQ ID NO: 5 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 5 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 5.

In some embodiments, the guide nucleic acid comprises, from 5′ to 3′, one said scaffold sequence, one said guide sequence, and one said scaffold sequence, wherein scaffold sequences are the same or different.

In some embodiments, the guide nucleic acid comprises, from 5′ to 3′, one said scaffold sequence, one said guide sequence, one said scaffold sequence, one said guide sequence, and one said scaffold sequence, wherein scaffold sequences are the same or different, and wherein guide sequences are the same or different.

In some embodiments, the guide nucleic acid comprises a sequence having a sequence identity of at least about 80% (e.g., at least about 80%, 85%, 90%, 91%, 92%, 93%, 94%, 95%, 96%, 97%, 98%, 99%, or 100%) to the sequence of any one of SEQ ID NOs: 17 and 30-31; or a sequence having at most 1, 2, 3, 4, 5, 6, 7, 8, 9, or 10 nucleotide differences, whether consecutive or not, compared to the sequence of any one of SEQ ID NOs: 17 and 30-31.

In some embodiments, the transcript of the VEGFA gene is a VEGFA pre-mRNA or a VEGFA mRNA.

In some embodiments, the VEGFA gene is human VEGFA gene.

In another aspect, the disclosure provides a recombinant adeno-associated virus (rAAV) vector genome comprising:
(1) a first polynucleotide sequence comprising a sequence comprising or encoding the guide nucleic acid of the disclosure; and
(2) a second polynucleotide sequence comprising a sequence encoding the Cas13 endonuclease of the disclosure,
   wherein the rAAV vector genome is adapted to be encapsulated into a rAAV particle.

In some embodiments, the first polynucleotide sequence comprises a U6 promoter (e.g., SEQ ID NO: 4 or 22) operably linked to the sequence comprising or encoding the guide nucleic acid.

In some embodiments, the second polynucleotide sequence comprises a Cbh promoter (e.g., SEQ ID NO: 6) operably linked to the sequence encoding the Cas13 endonuclease.

In some embodiments, the second polynucleotide comprises a first sequence encoding a first NLS at the 5′ end of the sequence encoding the Cas13 endonuclease and/or a second sequence encoding a second NLS at the 3′ end of the sequence encoding the Cas13 endonuclease.

In some embodiments, the first NLS and/or the second NLS is a SV40 NLS (e.g., SEQ ID NO: 26), a bpSV40 NLS (e.g., SEQ ID NO: 15), or a Nucleoplasmin NLS (npNLS).

In some embodiments, the second polynucleotide sequence comprises a WPRE sequence downstream of the sequence encoding the Cas13 endonuclease; optionally wherein the WPRE sequence is selected from the group consisting of Woodchuck Hepatitis Virus (WHP) Posttranscriptional Regulatory Element (WPRE), WPRE3 (e.g., SEQ ID NO: 11), and a functional variant (e.g., a functional truncation) thereof.

In some embodiments, the second polynucleotide sequence comprises a sequence encoding a bGH polyA signal (e.g., SEQ ID NO: 12) downstream of the sequence encoding the Cas13 endonuclease.

In some embodiments, the second polynucleotide sequence comprises, from 5′ to 3′, the promoter of SEQ ID NO: 6, Kozak sequence of gccacc, an in-frame start codon ATG, the first sequence encoding the first NLS of SEQ ID NO: 15, the sequence encoding the Cas13 endonuclease of SEQ ID NO: 16 or 32, the second sequence encoding the second NLS of SEQ ID NO: 15, an in-frame stop codon, the WPRE sequence of SEQ ID NO: 11, and the sequence encoding the polyA signal of SEQ ID NO: 12.

In some embodiments, the rAAV vector genome comprises, from 5′ to 3′, the first polynucleotide sequence and the second polynucleotide sequence; or comprises, from 5′ to 3′, the second polynucleotide sequence and the first polynucleotide sequence.

In some embodiments, the rAAV vector genome comprises, from the 5′ to 3′,
(1) the 5′ ITR of SEQ ID NO: 3;
(2) the promoter of SEQ ID NO: 4;
(3) a sequence encoding the scaffold sequence of SEQ ID NO: 5;
(4) a sequence encoding the guide sequence of SEQ ID NO: 1;
(5) a sequence encoding the scaffold sequence of SEQ ID NO: 5;
(6) a sequence encoding the guide sequence of SEQ ID NO: 2;
(7) a sequence encoding the scaffold sequence of SEQ ID NO: 5;
(8) the promoter of SEQ ID NO: 6;
(9) Kozak sequence of gccacc;
(10) in-frame start codon ATG;
(11) the first sequence of SEQ ID NO: 8 encoding the first NLS of SEQ ID NO: 15;
(12) the sequence of SEQ ID NO: 33 encoding the Cas13 endonuclease of SEQ ID NO: 32;
(13) the second sequence SEQ ID NO: 10 encoding the second NLS of SEQ ID NO: 15;
(14) an in-frame stop codon;
(15) the WPRE sequence of SEQ ID NO: 11;
(16) the sequence encoding a polyA signal of SEQ ID NO: 12; and
(17) the 3′ ITR of SEQ ID NO: 13;
   wherein the rAAV vector genome comprises an optional linker between any two adjacent components of the components (1) to (17).

In some embodiments, the rAAV vector genome comprises, consists essentially of, or consists of a polynucleotide having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 14.

In some embodiments, the rAAV vector genome comprises, from the 5′ to 3′,
(1) the 5′ ITR of SEQ ID NO: 3;
(2) the promoter of SEQ ID NO: 4;
(3) a sequence encoding the scaffold sequence of SEQ ID NO: 5;
(4) a sequence encoding the guide sequence of SEQ ID NO: 1;
(5) a sequence encoding the scaffold sequence of SEQ ID NO: 5;
(6) a sequence encoding the guide sequence of SEQ ID NO: 2;
(7) a sequence encoding the scaffold sequence of SEQ ID NO: 5;
(8) the promoter of SEQ ID NO: 6;
(9) Kozak sequence of gccacc;
(10) in-frame start codon ATG;
(11) the first sequence of SEQ ID NO: 8 encoding the first NLS of SEQ ID NO: 15;
(12) the sequence of SEQ ID NO: 33 encoding the Cas13 endonuclease of SEQ ID NO: 32;
(13) the second sequence SEQ ID NO: 10 encoding the second NLS of SEQ ID NO: 15;
(14) an in-frame stop codon;
(15) the WPRE sequence of SEQ ID NO: 11;
(16) the sequence encoding a polyA signal of SEQ ID NO: 12; and
(17) the 3′ ITR of SEQ ID NO: 13;
   wherein the rAAV vector genome comprises a sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 14;
   wherein the rAAV vector genome does not contain any nucleotide difference from the sequence of SEQ ID NO: 14 in any one of the components (1) to (17);
   wherein the rAAV vector genome contains one or more nucleotide difference or does not contain any nucleotide difference from the sequence of SEQ ID NO: 14 between any two adjacent components of the components (1) to (17).

In some embodiments, the rAAV vector genome comprises, consists essentially of, or consists of the sequence of SEQ ID NO: 14.

In yet another aspect, the disclosure provides a recombinant AAV (rAAV) particle comprising the rAAV vector genome of the disclosure.

In some embodiments, the rAAV particle comprising a capsid with a serotype of AAV8.

In some embodiments, the AAV8 is wild type AAV8 or a functional variant thereof.

In yet another aspect, the disclosure provides a method for production of a rAAV particle, comprising culturing in a host cell a transgene plasmid comprising the rAAV vector genome of the disclosure, thereby encapsulating the rAAV vector genome into a capsid with a serotype of AAV8.

In yet another aspect, the disclosure provides a cell comprising a transgene plasmid comprising the rAAV vector genome of the disclosure.

In yet another aspect, the disclosure provides use of the cell comprising a transgene plasmid comprising the rAAV vector genome of the disclosure for the production of rAAV particles.

In yet another aspect, the disclosure provides a pharmaceutical composition comprising (1) the system of the disclosure or the rAAV particle of the disclosure and (2) a pharmaceutically acceptable excipient.

In yet another aspect, the disclosure provides a method of downregulating expression of a VEGFA gene, comprising contacting the transcript of the VEGFA gene with the system of the disclosure, the rAAV particle of the disclosure, or the pharmaceutical composition of the disclosure.

In yet another aspect, the disclosure provides a cell or a progeny thereof comprising the system of the disclosure, or the rAAV particle of the disclosure.

In yet another aspect, the disclosure provides a cell or a progeny thereof comprising a VEGFA transcript modified by the system of the disclosure, the rAAV particle of the disclosure, or the method of the disclosure.

In yet another aspect, the disclosure provides a method for preventing, diagnosing, and/or treating a VEGFA associated disease in a subject in need thereof, comprising administering to the subject the system of the disclosure, the rAAV particle of the disclosure, or the pharmaceutical composition of the disclosure, wherein the Cas13 endonuclease modifies the transcript of VEGFA gene, leading to downregulated expression of VEGFA gene, thereby treating the VEGFA associated disease.

In some embodiments, the VEGFA associated disease is neovascular age-related macular degeneration (nAMD), Diabetic Macular Edema (DME), or a cancer.

In some embodiments, the subject is not responsive to anti-VEGF therapy, such as, anti-VEGF antibody.

The details of one or more embodiments of the disclosure are set forth in the description below. Other features or advantages of the disclosure will be apparent from the following drawings and detailed description of several embodiments, and also from the appended claims. It is understood that any aspect or embodiment of the disclosure can be combined with any other one or more aspects or embodiments of the disclosure, including aspects or embodiments only described in one sub-section, only in the examples, or only in the claims, to constitute another embodiment explicitly or implicitly disclosed herein unless otherwise indicated.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1 is a schematic showing an exemplary target dsDNA, an exemplary guide RNA, and an exemplary napDNAbp.

FIG. 2 is another schematic showing an exemplary target dsDNA, an exemplary guide RNA, and an exemplary napDNAbp.

FIG. 3 is a schematic showing an exemplary target dsDNA, an exemplary transcript (target RNA) transcribed from the target dsDNA, an exemplary guide RNA, and an exemplary napRNAbp.

FIG. 4 is another schematic showing an exemplary target dsDNA, an exemplary transcript (target RNA) transcribed from the target dsDNA, an exemplary guide RNA, and an exemplary napRNAbp, wherein the guide sequence contains a mismatch with the target sequence.

FIG. 5 shows comparison of in vivo VEGFA knockdown efficiency of HG202 and HG203.

FIG. 6 shows the results of VEGFA-targeting gRNA screening in vitro.

FIG. 7 shows in vitro expression and VEGFA knockdown of hfCas13Y and gRNA (SEQ ID NO: 17).

FIG. 8 shows off-target analysis in vitro of hfCas13Y and gRNA (SEQ ID NO: 17).

FIG. 9 shows VEGFA knockdown in vivo of HG202.

FIG. 10 shows reduction of CNV in vivo by HG202.

FIG. 11 shows safety of HG202.

FIG. 12 shows a representative high-fidelity Cas13Y (hfCas13Y) endonuclease based therapeutic system.

FIG. 13 shows schematic of animal modelling and treatment.

The figures herein are for illustrative purposes only and are not necessarily drawn to scale.

### Definitions

The disclosure will be described with respect to particular embodiments, but the disclosure is not limited thereto in any respect. Unless defined otherwise, all technical and scientific terms used herein have the same meaning as is commonly understood by one of ordinary skill in the art to which this disclosure belongs. Terms as set forth hereinafter are generally to be understood in their plain and ordinary meaning or common sense unless indicated otherwise.

### Overview

Nucleic acid programmable binding protein (napBP), for example, nucleic acid programmable DNA binding protein, (napDNAbp), such as Cas9, Cas12, IscB, nucleic acid programmable RNA binding protein (napRNAbp), such as, Cas13, is capable of binding to a target nucleic acid (e.g., dsDNA, mRNA) as guided by a guide nucleic acid (e.g., a guide RNA) comprising a guide sequence targeting the target nucleic acid. In some embodiments, the target nucleic acid is eukaryotic.

Without wishing to be bound by theory, in some embodiments, the guide nucleic acid comprises a scaffold sequence responsible for forming a complex with the napBP, and a guide sequence that is intentionally designed to be responsible for hybridizing to a target sequence of the target nucleic acid, thereby guiding the complex comprising the napBP and the guide nucleic acid to the target nucleic acid.

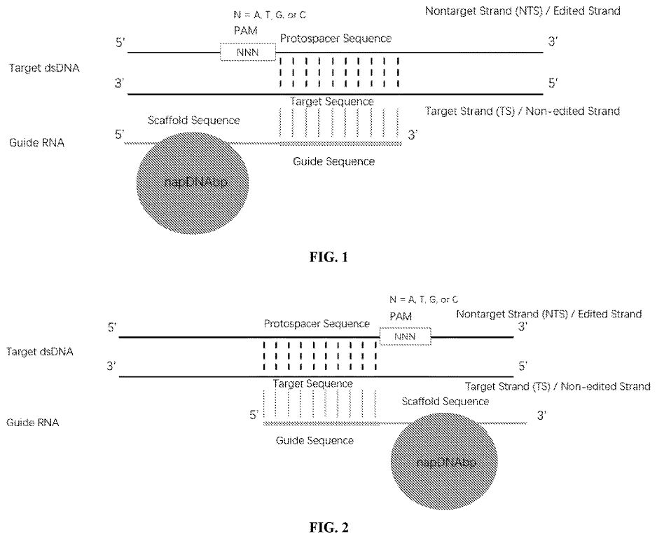

Referring to FIG. 1, an exemplary target dsDNA (e.g., VEGFA gene) is depicted to comprise a 5′ to 3′ single DNA strand and a 3′ to 5′ single DNA strand.

An exemplary guide nucleic acid (e.g., a guide RNA) is depicted to comprise a guide sequence and a scaffold sequence. The guide sequence is designed to hybridize to a part of the 3′ to 5′ single DNA strand, and so the guide sequence “targets” that part. And thus, the 3′ to 5′ single DNA strand is referred to as a “target strand (TS)” of the target dsDNA, while the opposite 5′ to 3′ single DNA strand is referred to as a “nontarget strand (NTS)” of the target dsDNA. That part of the target strand based on which the guide sequence is designed and to which the guide sequence may hybridize is referred to as a “target sequence”, while the opposite part on the nontarget strand corresponding to that part is referred to as the “protospacer sequence”, which is 100% (fully) reversely complementary to the target sequence and is said to be “corresponding to” the target sequence in the disclosure.

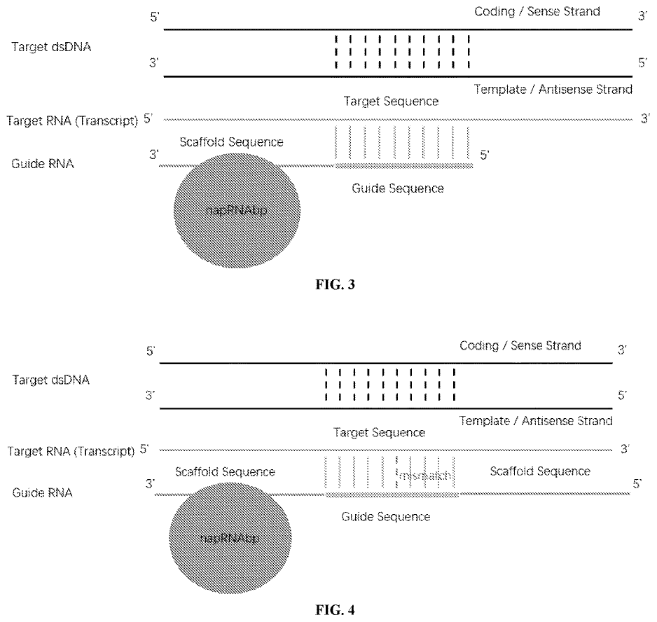

Referring to FIG. 3, an exemplary target dsDNA (e.g., VEGFA gene) is depicted to comprise a 5′ to 3′ single DNA strand and a 3′ to 5′ single DNA strand. According to conventional transcription process, an exemplary target RNA (transcript, e.g., a pre-mRNA) may be transcribed using the 3′ to 5′ single DNA strand as a synthesis template, and thus the 3′ to 5′ single DNA strand is referred to as a “template strand” or a “antisense strand”. The transcript so transcribed has the same primary sequence as the 5′ to 3′ single DNA strand except for the replacement of T with U, and thus the 5′ to 3′ single DNA strand is referred to as a “coding strand” or a “sense strand”.

An exemplary guide nucleic acid (e.g., a guide RNA) is depicted to comprise a guide sequence and a scaffold sequence. The guide sequence is designed to hybridize to a part of the transcript (target RNA), and so the guide sequence “targets” that part. And thus, that part of the target RNA based on which the guide sequence is designed and to which the guide sequence may hybridize is referred to as a “target sequence”. In some embodiments, the guide sequence is 100% (fully) reversely complementary to the target sequence. In some other embodiments, the guide sequence is reversely complementary to the target sequence and contains a mismatch with the target sequence (as exemplified in FIG. 4).

Generally, as is conventional in the art, a nucleic acid sequence (e.g., a DNA sequence, an RNA sequence) is written in 5′ to 3′ direction/orientation unless explicitly indicated otherwise.

For example, for a DNA sequence of ATGC, it is usually understood as 5′-ATGC-3′ unless otherwise indicated. Its reverse sequence is 5′-CGTA-3′. Its fully complementary sequence is 5′-TACG-3′. Its fully reverse complementary sequence is 5′-GCAT-3′. Note that the fully complementary sequence usually does not have the ability to base-pair/hybridize with the original sequence.

Generally, the double-strand sequence of a dsDNA may be represented with the sequence of its 5′ to 3′ single DNA strand conventionally written in 5′ to 3′ direction/orientation unless otherwise indicated.

For example, for a dsDNA having a 5′ to 3′ single DNA strand of 5′-ATGC-3′ and a 3′ to 5′ single DNA strand of 3′-TACG-5′, the dsDNA may be simply represented as 5′-ATGC-3′.

5′-----ATGC-----3′
3′-----TACG-----5′

It should be noted that either the 5′ to 3′ single DNA strand or the 3′ to 5′ single DNA strand of a dsDNA can be a nontarget strand from which a protospacer sequence is selected.

Generally, for a gene as a dsDNA, the 5′ to 3′ single DNA strand is the sense strand of the gene, and the 3′ to 5′ single DNA strand is the antisense strand of the gene. It should be noted that either the sense strand or the antisense strand of a gene can be a nontarget strand from which a protospacer sequence is selected.

Normally, the transcript (target RNA) transcribed from the dsDNA then has a (target) sequence of 5′-AUGC-3′.

To hybridize to a target dsDNA, in one embodiment, the guide sequence of a guide nucleic acid is designed to have a sequence of 5′-AUGC-3′ that is fully reversely complementary to the 3′ to 5′ strand of the target dsRNA, which would be set forth in ATGC in the electric sequence listing but marked as an RNA sequence; and in another embodiment, the guide sequence of a guide nucleic acid is designed to have a sequence of 5′-GCAU-3′ that is fully reversely complementary to the 5′ to 3′ strand of the target dsRNA, which would be set forth in GCAT in the electric sequence listing but marked as an RNA sequence.

In the case that the guide sequence of a guide nucleic acid is fully reversely complementary to the target sequence and the target sequence is fully reversely complementary to the protospacer sequence, the guide sequence is identical to the protospacer sequence except for the U in the guide sequence due to its RNA nature and correspondingly the T in the protospacer sequence due to its DNA nature. According to WIPO standard ST.26, symbol “t” is used to denote both T in DNA and U in RNA (See “Table 1: List of nucleotides symbols”, the definition of symbol “t” is “thymine in DNA/uracil in RNA (t/u)”). Thus, in the electronic sequence listing of the disclosure prepared according to ST.26, such a guide sequence could be set forth in the same sequence as a corresponding protospacer sequence. For convenience, a single SEQ ID NO in the electronic sequence listing can be used to denote both such guide sequence and protospacer sequence, regardless whether such a single SEQ ID NO is marked as DNA or RNA in the electronic sequence listing. When a reference is made to such a SEQ ID NO that sets forth a protospacer/guide sequence, it refers to either a protospacer sequence that is a DNA sequence or a guide sequence that is an RNA sequence depending on the context, no matter whether it is marked as a DNA or an RNA in the electronic sequence listing.

To hybridize to the target RNA, in one embodiment, the guide sequence of a guide nucleic acid is designed to have a sequence of 5′-GCAU-3′ that is fully reversely complementary to the (target) sequence of the target RNA, which would be set forth in GCAT in the electric sequence listing but marked as an RNA sequence.

### Term

As used herein, if a DNA sequence, for example, 5′-ATGC-3′ is transcribed to an RNA sequence, with each dT (deoxythymidine, or “T” for short) in the primary sequence replaced with a U (uridine) and other dA (deoxyadenosine, or “A” for short), dG (deoxyguanosine, or “G” for short), and dC (deoxycytidine, or “C” for short) replaced with A (adenosine), G (guanosine), and C (cytidine), respectively, for example, 5′-AUGC-3′, it is said in the disclosure that the DNA sequence “encodes” the RNA sequence.

As used herein, the term “activity” refers to a biological activity. In some embodiments, the activity includes enzymatic activity, e.g., catalytic ability of an effector. For example, the activity can include nuclease activity, e.g., dsDNA endonuclease activity, RNA endonuclease activity.

As used herein, the term “nucleic acid programmable binding protein (napBP)” may be used interchangeably with “nucleic acid programmable binding domain (napBD)” to refer to a protein that can associate (e.g., bind) with a programmable nucleic acid (e.g., DNA or RNA), such as a guide nucleic acid (e.g., gRNA), that is able to be programmed to guide the protein to a specific sequence of a target nucleic acid via the interaction (e.g., hybridization) between the programmable nucleic acid (e.g., the guide sequence of the programmable nucleic acid) and the target nucleic acid (e.g., the target sequence of the target nucleic acid). The napBP may be indirectly associated with (e.g., bound to) the target nucleic acid via the interaction (e.g., binding) between the napBP and the programmable nucleic acid (e.g., scaffold sequence of the programmable nucleic acid) and the interaction (e.g., hybridization) between the programmable nucleic acid (e.g., the guide sequence of the programmable nucleic acid) and the target nucleic acid (e.g., the target sequence of the target nucleic acid). In some embodiments, the napBP is a nucleic acid programmable DNA binding protein (napDNAbp). In some embodiments, the napBP is a nucleic acid programmable RNA binding protein (napRNAbp).

As used herein, the term “complex” refers to a grouping of two or more molecules. In some embodiments, the complex comprises a polypeptide and a nucleic acid interacting with (e.g., binding to, coming into contact with, adhering to) one another. As used herein, the term “complex” can refer to a grouping of a guide nucleic acid and a polypeptide (e.g., a napBP). As used herein, the term “complex” can refer to a grouping of a guide nucleic acid, a polypeptide (e.g., a napBP), and a target nucleic acid.

As used herein, the term “protospacer adjacent motif” or “PAM” refers to a short DNA sequence (or a DNA motif) adjacent to a protospacer sequence on the nontarget strand of a dsDNA. As used herein, the term “adjacent” includes instances wherein there is no nucleotide between the protospacer sequence and the PAM and also instances wherein there are a small number (e.g., 1, 2, 3, 4, or 5) of nucleotides between the protospacer sequence and the PAM. As used herein, A “immediately adjacent (to)” B, A “immediately 5′ to” B, and A “immediately 3′ to” B mean that there is no nucleotide between A and B. In some embodiments, the PAM is immediately 5′ to a protospacer sequence. In some embodiments, the PAM is immediately 3′ to a protospacer sequence.

As used herein, the term “guide nucleic acid” refers to any nucleic acid that facilitates the targeting of a napBP to a target nucleic acid. For this purpose, the guide nucleic acid may be designed to include a guide sequence capable of hybridizing to a specific sequence of a target nucleic acid, and the guide nucleic acid may also comprise a scaffold sequence facilitating the guiding of a napBP to the target nucleic acid. In some embodiments, the guide nucleic acid is a guide RNA. In some embodiments, the guide nucleic acid is a nucleic acid encoding a guide RNA.

As used herein, the terms “nucleic acid”, “polynucleotide”, and “nucleotide sequence” are used interchangeably to refer to a polymeric form of nucleotides of any length, including deoxyribonucleotides, ribonucleotides, combinations thereof, and analogs or modifications thereof.

As used in the context of CRISPR-Cas techniques (e.g., CRISPR-Cas12 techniques), the term “guide RNA” is used interchangeably with the term “CRISPR RNA (crRNA)”, “single guide RNA (sgRNA)”, or “RNA guide”, the term “guide sequence” is used interchangeably with the term “spacer sequence”, and the term “scaffold sequence” is used interchangeably with the term “direct repeat sequence”.

As described herein, the guide sequence is so designed to be capable of hybridizing to a target sequence. As used herein, the term “hybridize”, “hybridizing”, or “hybridization” refers to a reaction in which one or more polynucleotide sequences react to form a complex that is stabilized via hydrogen bonding between the bases of the polynucleotide sequences. The hydrogen bonding may occur by Watson Crick base pairing, Hoogstein binding, or in any other sequence specific manner. A polynucleotide sequence capable of hybridizing to a given polynucleotide sequence is referred to as the “complement” of the given polynucleotide sequence. As used herein, the hybridization of a guide sequence and a target sequence is so stabilized to permit an effector polypeptide (e.g., a napBP) that is complexed with a nucleic acid comprising the guide sequence or a function domain associated (e.g., fused) with the effector polypeptide to act (e.g., cleave, deaminize) on the target sequence or its complement or nearby sequence.

For the purpose of hybridization, in some embodiments, the guide sequence is reversely complementary to a target sequence. As used herein, the term “reverse complementary” refers to the ability of nucleobases of a first polynucleotide sequence, such as a guide sequence, to base pair with nucleobases of a second polynucleotide sequence, such as a target sequence, by traditional Watson-Crick base-pairing. Two reverse complementary polynucleotide sequences are able to non-covalently bind under appropriate temperature and solution ionic strength conditions. In some embodiments, a first polynucleotide sequence (e.g., a guide sequence) comprises 100% (fully) reverse complementarity to a second nucleic acid (e.g., a target sequence). In some embodiments, a first polynucleotide sequence (e.g., a guide sequence) is reverse complementary to a second polynucleotide sequence (e.g., a target sequence) if the first polynucleotide sequence comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid (i.e., at least about 80%, 81%, 82%, 83%, 84%, 85%, 86%, 87%, 88%, 89%, 90%, 91%, 92%, 93%, 94%, 95%, 96%, 97%, 98%, or 99% of the nucleotides of the first polynucleotide sequence can base-pair with the nucleotides of the second polynucleotide sequence). As used herein, the term “substantially complementary” refers to a first polynucleotide sequence (e.g., a guide sequence) that has a certain level of complementarity to a second polynucleotide sequence (e.g., a target sequence) (e.g., at least about 80%, 81%, 82%, 83%, 84%, 85%, 86%, 87%, 88%, 89%, 90%, 91%, 92%, 93%, 94%, 95%, 96%, 97%, 98%, or 99% of the nucleotides of the first polynucleotide sequence can base-pair with the nucleotides of the second polynucleotide sequence, or at most 1, 2, 3, 4, or 5 contiguous or non-contiguous nucleotides of the first polynucleotide sequence mismatch the nucleotides of the second polynucleotide sequence). In some embodiments, the level of complementarity is such that the first polynucleotide sequence (e.g., a guide sequence) can hybridize to the second polynucleotide sequence (e.g., a target sequence) with sufficient affinity to permit an effector polypeptide (e.g., a napBP) that is complexed with a nucleic acid comprising the first polynucleotide sequence or a function domain associated (e.g., fused) with the effector polypeptide to act (e.g., cleave, deaminize) on the target sequence or its complement or nearby sequence. In some embodiments, a guide sequence that is substantially complementary to a target sequence has less than 100% complementarity to the target sequence. In some embodiments, a guide sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence, and/or has at most 1, 2, 3, 4, or 5 contiguous or non-contiguous nucleotide mismatches from the target sequence.

As used herein, the term “sequence identity” is related to sequence homology. Homology comparisons may be conducted by eye, or more usually, with the aid of readily available sequence comparison programs. These commercially available computer programs may calculate percentage sequence identity (%) between two or more sequences (polypeptide or polynucleotide sequences). Sequence homologies may be generated by any of a number of computer programs known in the art, for example, BLAST, FASTA. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410), and the GENEWORKS suite of comparison tools. Both BLAST and FASTA are available for offline and online searching (see Ausubel et al., 1999 ibid, pages 7-58 to 7-60). A commonly used online tool to calculate percentage sequence identity between two or more sequences (polypeptide or polynucleotide sequences) is available on the website of EMBL's European Bioinformatics Institute (www dot ebi dot ac dot uk slash jdispatcher slash), allowing fast online calculation of percentage sequence identity by global alignment or local alignment.

As used herein, the terms “polypeptide” and “peptide” are used interchangeably herein to refer to polymers of amino acids of any length. A protein may have one or more polypeptides. An amino acid polymer can also be modified, for example, by disulfide bond formation, glycosylation, lipidation, acetylation, phosphorylation, or any other manipulation, such as conjugation with a labeling component.

As used herein, a “variant” is interpreted to mean a polynucleotide or polypeptide that differs from a reference polynucleotide or polypeptide, respectively, but retains essential properties, e.g., binding property of a napBP. A typical variant of a polynucleotide differs in nucleic acid sequence from another reference polynucleotide. A change in the nucleic acid sequence of the polynucleotide variant may or may not alter the amino acid sequence of a polypeptide encoded by the reference polynucleotide. A change in the nucleic acid sequence of the polynucleotide variant may result in an amino acid substitution, addition, and/or deletion in the polypeptide encoded by the reference polynucleotide. A typical variant of a polypeptide differs in amino acid sequence from another reference polypeptide. Generally, the difference is limited so that the sequences of the reference polypeptide and the polypeptide variant are closely similar overall and, in many regions, identical. The polypeptide variant and reference polypeptide may differ in amino acid sequence by one or more substitutions, additions, and/or deletions in any combination. A variant of a polynucleotide or polypeptide may be naturally occurring, such as, an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the terms “upstream” and “downstream” refer to the relative positions of two or more elements within a nucleic acid in 5′ to 3′ direction. A first sequence is upstream of a second sequence when the 3′ end of the first sequence is present at the left side of the 5′ end of the second sequence. A first sequence is downstream of a second sequence when the 5′ end of the first sequence is present at the right side of the 3′ end of the second sequence. In some embodiments, the PAM is upstream of a napBP-induced indel, and a napBP-induced indel is downstream of the PAM. In some embodiments, the PAM is downstream of a napBP-induced indel, and a napBP-induced indel is upstream of the PAM.

As used herein, the term “wild type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the terms “non-naturally occurring” and “engineered” are used interchangeably and refer to artificial participation. When these terms are used to describe a nucleic acid or a polypeptide, it is meant that the nucleic acid or polypeptide is at least substantially freed from at least one other component of its association in nature or as found in nature.

As used herein, the term “regulatory element” is intended to include promoters, enhancers, internal ribosome entry sites (IRES), and other expression control elements (e.g., transcription termination signals, such as, polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cells and those that direct expression of a nucleotide sequence only in certain host cells (e.g., tissue-specific regulatory sequences). Regulatory elements may also direct expression in a time-dependent manner, e.g., in a cell cycle-dependent or developmental stage-dependent manner, which may or may not be tissue or cell type specific.

As used herein, the term “cell” is understood to refer not only to a particular individual cell, but to the progeny or potential progeny of the cell. Because certain modifications may occur in succeeding generations due to either mutation or environmental influences, such progeny may not, in fact, be identical to the parent cell, but are still included within the scope of the term.

As used herein, the term “in vivo” refers to inside the body of an organism, and the terms “ex vivo” or “in vitro” means outside the body of an organism.

As used herein, the term “treat”, “treatment”, or “treating” is an approach for obtaining beneficial or desired results including clinical results. For purposes of the disclosure, the beneficial or desired clinical results include, but are not limited to, one or more of the following: alleviating one or more symptoms resulting from a disease, diminishing the extent of a disease, stabilizing a disease (e.g., delaying the worsening of a disease), delaying the spread (e.g., metastasis) of a disease, delaying the recurrence of a disease, reducing recurrence rate of a disease, delay or slowing the progression of a disease, ameliorating a disease state, providing a remission (partial or total) of a disease, decreasing the dose of one or more other medications required to treat a disease, delaying the progression of a disease, increasing the quality of life, and/or prolonging survival. Also encompassed by “treatment” is a reduction of pathological consequence of a disease (such as cancer). The methods of the disclosure contemplate any one or more of these aspects of treatment.

As used herein, the term “disease” includes the terms “disorder” and “condition” and is not limited to those have been specifically medically defined.

As used herein, reference to “not” a value or parameter generally means and describes “other than” a value or parameter. For example, the method is not used to treat cancer of type X means the method may be used to treat cancer of types other than X.

As used herein, the singular forms “a”, “an”, and “the” include plural referents unless the context clearly dictates otherwise.

As used herein, the term “and/or” in a phrase such as “A and/or B” is intended to include both A and B; A or B; A (alone); and B (alone). Likewise, the term “and/or” in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, when the term “about” is ahead of a serious of numbers (for example, about 1, 2, 3), it is understood that each of the serious of numbers is modified by the term “about” (that is, about 1, about 2, about 3). The term “about X-Y” or “about X to Y” used herein has the same meaning as “about X to about Y.”

It is understood that embodiments of the disclosure described herein include “consisting” and/or “consisting essentially of” embodiments.

It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

### DETAILED DESCRIPTION

### Overview

VEGFA gene (also known as VPF, VEGF, or MVCD1) encodes vascular endothelial growth factor A (VEGFA), which plays a vital role in both physiological development of vascular structures and pathological angiogenesis. It has been reported that VEGFA is associated with diseases, such as, neovascular age-related macular degeneration (nAMD) (also known as wet AMD) and Diabetic Macular Edema (DME). AMD is a major cause of visual dysfunction for adults over age 50 of which nAMD accounted for 90% blindness in AMD. Choroidal neovascularization (CNV) is the hallmark of nAMD. Overexpression of VEGFA promotes CNV. WO2022/188797 (PCT/CN2022/079890), incorporated herein by reference in its entirety, discloses knockdown of VEGFA mRNA by AAV9-delivered hfCas13X.1 (also known as hfCas13e.1) system. It would be desired to develop tools and methods to knockdown VEGFA mRNA and/or treat VEGFA associated diseases with improved efficiency and/or safety.

In some aspects, the disclosure provides tools and methods for treating VEGFA associated diseases, such as, nAMD and DME.

As used herein, the term “VEGFA associated diseases” includes diseases that are associated with VEGFA gene, the transcription (including transcript; e.g., increased or decreased transcription, nonfunctional or dysfunctional transcript) thereof, and/or the expression (including expression and expression product; e.g., increased or decreased expression, nonfunctional or dysfunctional expression product) thereof, including those that are directly or indirectly caused by the transcription (including transcription and transcript, e.g., VEGFA mRNA) of VEGFA gene, and those that are directly or indirectly caused by the expression (including expression product, e.g., vascular endothelial growth factor A) of VEGFA gene. In some embodiments, the term “VEGFA associated disease” refers to a disease that is caused by abnormal expression (e.g., overexpression or deficient expression) of VEGFA gene. In some embodiments, the term “VEGFA associated disease” refers to a disease that is caused by the deficiency of a functional expression product of VEGFA gene, e.g., vascular endothelial growth factor A.

As used herein, the term “transcript” includes any transcription product by transcription from a gene, including mRNA, non-coding RNA, and any variants, derivatives, or ancestors thereof, for example, pre-mRNA, and any transcripts or isoforms produced from the gene or the pre-mRNA by, e.g., alternative promoter usage, alternative splicing, alternative initiation, and any naturally occurring variants thereof or processed products therefrom. The transcript of the VEGFA gene is also termed as VEGFA transcript in the disclosure.

In some embodiments, the VEGFA gene is human VEGFA gene. Human wild type VEGFA gene has NCBI Gene ID: 7422. In some embodiments, the transcript is human VEGFA mRNA. Human VEGFA mRNA has NCBI Accession No.: NM_001025366. In some embodiments, the expression product is human vascular endothelial growth factor A. Human vascular endothelial growth factor A has NCBI Accession No.: NP_001020537.

The disclosure provides, in some embodiments, tools for decreasing/downregulating the expression of VEGFA gene by cleaving (e.g., relying on the function of Cas13 endonuclease) (knocking down) VEGFA transcript (e.g., VEGFA mRNA). The tools can be delivered, for example, by rAAV vectors to subjects in need. In some embodiments, the tools include guide nucleic acids comprising a guide sequence designed to be capable of hybridizing to VEGFA mRNA, thereby guiding a complex comprising the guide nucleic acid and a napRNAn to the VEGFA mRNA and enabling the napRNAn to modify (specifically cleave) the VEGFA mRNA, leading to degradation of the VEGFA mRNA. In the case that VEGFA expression is properly downregulated, it is reasonably expected that the syndromes (e.g., syndromes of nAMD) caused by the abnormal VEGFA expression can be alleviated.

### Guide Nucleic Acid

In an aspect, the disclosure provides a guide nucleic acid comprising a guide sequence capable of hybridizing to a target sequence on a target strand of a VEGFA gene or a target sequence on a transcript of a VEGFA gene.

In some embodiments, the guide nucleic acid comprises a scaffold sequence capable of forming a complex with a nucleic acid programmable binding protein (napBP), and wherein the hybridization of the guide sequence to the target sequence guides the complex to the VEGFA gene or the transcript.

The components of the guide nucleic acid are described more specifically in the other sub-sections herein.

### System

In another aspect, the disclosure provides a system comprising:
(1) a guide nucleic acid, or a polynucleotide (e.g., a DNA, an RNA, a DNA/RNA mixture) encoding the guide nucleic acid, comprising:
   (a) a scaffold sequence capable of forming a complex with a nucleic acid programmable binding protein (napBP), and
   (b) a guide sequence capable of hybridizing to a target sequence on a target strand of a VEGFA gene or a target sequence on a transcript of a VEGFA gene, thereby guiding the complex to the VEGFA gene or the transcript; and
(2) the napBP or a polynucleotide encoding the napBP.

In some embodiments, the system is a complex comprising the napBP complexed with the guide nucleic acid. In some embodiments, the complex further comprises the VEGFA gene or transcript thereof hybridized with the guide sequence.

In some embodiments, the system is a composition comprising the component (1) and the component (2).

The components of the system are described more specifically in the other sub-sections herein.

rAAV Vector Genome

The disclosure provides various delivery of the system of the disclosure, for example, delivery via a rAAV vector. In some embodiments, the disclosure provides a recombinant adeno-associated virus (rAAV) vector genome encoding or comprising the system of the disclosure. In some embodiments, the rAAV vector genome is a DNA (e.g., a ssDNA, a dsDNA) or an RNA.

Thus, in yet another aspect, the disclosure provides a recombinant adeno-associated virus (rAAV) vector genome (e.g., a DNA rAAV vector genome, an RNA rAAV vector genome) comprising:
(1) a first polynucleotide sequence comprising a sequence encoding a guide nucleic acid comprising:
   (a) a scaffold sequence capable of forming a complex with a nucleic acid programmable binding protein (napBP), and
   (b) a guide sequence capable of hybridizing to a target sequence on a target strand of a VEGFA gene or a target sequence on a transcript of a VEGFA gene, thereby guiding the complex to the VEGFA gene or the transcript; and
(2) a second polynucleotide sequence comprising a sequence encoding the napBP,
   wherein the rAAV vector genome is adapted to be encapsulated into a rAAV particle (e.g., a DNA-encapsulated rAAV particle, an RNA-encapsulated rAAV particle).

The components of the rAAV vector genome are described more specifically in the other sub-sections herein.

### Modification Method

In yet another aspect, the disclosure provides a method of modifying (e.g., downregulating) expression of a VEGFA gene, comprising contacting the VEGFA gene or transcript thereof with a system comprising:
(1) a guide nucleic acid, or a polynucleotide (e.g., a DNA, an RNA, a DNA/RNA mixture) encoding the guide nucleic acid, comprising:
   (a) a scaffold sequence capable of forming a complex with a nucleic acid programmable binding protein (napBP), and
   (b) a guide sequence capable of hybridizing to a target sequence on a target strand of a VEGFA gene or a target sequence on a transcript of a VEGFA gene, thereby guiding the complex to the VEGFA gene or the transcript and modifying the expression of the VEGFA gene; and
(2) the napBP or a polynucleotide encoding the napBP.

In some embodiments, the modification of the expression of the VEGFA gene treats, detects, or diagnose a VEGFA associated disease. In some embodiments, the modification of the expression of the VEGFA gene detects or diagnose the presence, progress, or development of a VEGFA associated disease.

The components of the method are described more specifically in the other sub-sections herein.

### Mechanism

The disclosure provides various mechanisms for modifying expression of a VEGFA gene for various purposes, including, but not limited to, treatment or diagnosis of VEGFA associated diseases. The modification could be on gene level, transcription level, transcript level, or translation level. In some embodiments, the modification leads to downregulated expression of VEGFA gene so as to treat VEGFA associated diseases.

As used herein, the phrase “modifying expression of a VEGFA gene” includes, but not limited to, modifications of any one or more stages of the transcription and translation procedure starting from the VEGFA gene to an expression product of the VEGFA gene, e.g., vascular endothelial growth factor A. Non-limiting examples of the modifications include:
(1) modifying the VEGFA gene, such as, cleaving the VEGFA gene (and thereby introducing an indel mutation into the VEGFA gene), changing (e.g., adding, deleting, substituting) any one or more nucleotides of the VEGFA gene by, for example, base editing, prime editing;
(2) modifying the transcription of the VEGFA gene, such as, increasing or decreasing the transcription by, for example, methylating or demethylating the VEGFA gene by epigenetic modification;
(3) modifying the transcript of the VEGFA gene, such as, a VEGFA mRNA, for example, cleaving (and typically degrading) the transcript, changing (e.g., adding, deleting, substituting) any one or more nucleotides of the transcript by, for example, base editing; and
(4) modifying the translation of the transcript (e.g., a VEGFA mRNA) of the VEGFA gene, such as, increasing or decreasing the translation of the transcript by, for example, epigenetic modification.

### DNA Modification

Provided in the disclosure are multiple ways to modify the expression of VEGFA gene on DNA level. In some embodiments, the VEGFA gene comprises a mutation. In some embodiments, the VEGFA gene is a human VEGFA gene comprising a mutation. Various VEGFA mutation that causes a VEGFA associated disease is known in the art. In some embodiments, the napBP is a nucleic acid programmable DNA binding protein (napDNAbp).

### DNA Cleavage

Provided in the disclosure are tools and methods using DNA cleavage to modify the expression of VEGFA gene.

In some embodiments, the napDNAbp is a nucleic acid programmable dsDNA endonuclease (napDNAn).

In some embodiments, guiding the complex to the VEGFA gene enables the napDNAn to specifically cleave the VEGFA gene in a guide sequence-specific manner.

In some embodiments, the specific cleavage of the VEGFA gene leads to incorporation of an insertion and/or deletion (an indel) mutation into the VEGFA gene.

In some embodiments, the specific cleavage of the VEGFA gene or the insertion and/or deletion mutation generates an in-frame stop codon in the VEGFA gene. As is well understood, the presence of an in-frame stop codon in a gene and/or in the transcript transcribed from a gene typically stops the translation of a protein from the transcript. Typically, stop codon is TAG, TAA, or TGA in DNA, and UAG, UAA, or UGA in RNA.

In some embodiments, the specific cleavage of the VEGFA gene or the insertion and/or deletion mutation generates a 3n+1 frameshift mutation, a 3n+2 frameshift mutation, a 3n−1 frameshift mutation, or a 3n−2 frameshift mutation in the VEGFA gene, wherein n is 0 or a positive integer (e.g., 1, 2, 3).

In some embodiments, the frameshift mutation decreases or eliminates transcription of the VEGFA gene and/or translation of a transcript (e.g., mRNA) of the VEGFA gene. In some embodiments, the frameshift mutation generates an in-frame stop codon in the VEGFA gene.

In some embodiments, the frameshift mutation leads to expression of a mutant of vascular endothelial growth factor A. In some embodiments, the mutant is functional. In some embodiments, the mutant is non-functional.

In some embodiments, the frameshift mutation leads to expression of a functional mutant of vascular endothelial growth factor A. As used herein, the term “functional mutant” of a protein refers to a mutant of the protein that substantially has one or more functions (e.g., all known functions) of the protein, for example, a mutant having at least about 35%, 40%, 45%, 50%, 55%, 60%, 65%, 70%, 75%, 80%, 85%, 90%, or 95% of the function of the protein or having about 100% function of the protein.

In some embodiments, the frameshift mutation leads to expression of a non-functional mutant of vascular endothelial growth factor A. As used herein, the term “non-functional mutant” of a protein refers to a mutant of the protein that substantially lacks one or more functions (e.g., all known functions) of the protein, for example, a mutant having at most about 35%, 30%, 20%, 15%, 10%, 5%, or 1% of the function of the protein or having about 0% function of the protein.

In some embodiments, the frameshift mutation leads to expression of a non-pathogenic mutant of vascular endothelial growth factor A. As used herein, the term “non-pathogenic mutant” of a protein refers to a mutant of the protein that is not pathogenic or has not been reported to cause any disease.

In some embodiments, the frameshift mutation does not lead to expression of a pathogenic mutant of vascular endothelial growth factor A. As used herein, the term “pathogenic mutant” of a protein refers to a mutant of the protein that is pathogenic or has been reported to cause any disease.

In some embodiments, the system of disclosure is combined with a supplement to deliver the wild type expression product of the VEGFA gene. For example, in some embodiments, a cDNA encoding the wild type expression product of VEGFA gene is delivered in combination with the system of disclosure delivered in rAAV particles in the same or separate rAAV particles.

### DNA Base Editing

Also provided in the disclosure are tools and methods using DNA base editing to modify the expression of VEGFA gene.

In some embodiments, the napDNAbp is a nickase and further comprises a base editing domain. As used herein, the term “nickase” means that it substantially lacks dsDNA endonuclease activity (i.e., it is substantially incapable of cleaving both strands of a dsDNA) and is substantially capable of nicking one strand of a dsDNA. In some embodiments, the one strand is the target strand of the dsDNA. In some embodiments, the one strand is the sense strand or the antisense strand of the dsDNA.

In some embodiments, the napDNAbp is an endonuclease deficient napDNAbp and further comprises a base editing domain. As used herein, the term “endonuclease deficient napDNAbp” is used interchangeably with “endonuclease inactive napDNAbp” or “dead napDNAbp”, meaning that it substantially lacks dsDNA endonuclease activity (i.e., it is substantially incapable of cleaving double strands of a dsDNA), and it substantially lacks nickase activity (i.e., it is substantially incapable of nicking either strand of a dsDNA).

In some embodiments, guiding the complex to the VEGFA gene enables the napDNAbp that is a nickase or dead napDNAbp and further comprises the base editing domain to specifically base edit the VEGFA gene in a guide sequence-specific manner.

As used herein, the term “base edit(ing)” means editing the base of a nucleotide (e.g., a ribonucleotide, a deoxyribonucleotide), including but not limited to inserting (adding), deleting (excising), and substituting the base.

In some embodiments, the base editing of the VEGFA gene substitutes a nucleotide of the VEGFA gene to a different nucleotide. In some embodiments, the base editing of the VEGFA gene substitutes one nucleotide of the VEGFA gene to one different nucleotide, which is also known as single base editing. In some embodiments, the base editing of the VEGFA gene substitutes two or three or more nucleotides, whether consecutive or not, of the VEGFA gene to two or three or more different nucleotides, which is also known as multiple or multiplexed base editing.

As well known in the art and shown in the tables below, codon includes 61 non-stop codons encoding 22 amino acids and three stop codons (i.e., TAA, TAG, TGA). Each codon is composed of three nucleotides. A nucleotide change in a non-stop codon may convert the non-stop codon to another non-stop codon encoding the same or different amino acid, or to a stop codon. A nucleotide change in a stop codon may convert the stop codon or another stop codon, or to a non-stop codon. Unless otherwise indicated, the codon in the disclosure, e.g., a non-stop codon, a stop codon, a start codon, is an in-frame codon.

Nucleotide position in codon

| | Second | | | | |
|---|---|---|---|---|---|
| First | T | C | A | G | Third |
| T | TTT - Phe | TCT - Ser | TAT - Tyr | TGT - Cys | T |
| | TTC - Phe | TCC - Ser | TAC - Tyr | TGC - Cys | C |
| | TTA - Leu | TCA - Ser | TAA - * | TGA - * | A |
| | TTG - Leu | TCG - Ser | TAG - * | TGG - Trp | G |
| C | CTT - Leu | CCT - Pro | CAT - His | CGT - Arg | T |
| | CTC - Leu | CCC - Pro | CAC - His | CGC - Arg | C |
| | CTA - Leu | CCA - Pro | CAA - Gln | CGA - Arg | A |
| | CTG - Leu | CCG - Pro | CAG - Gln | CGG - Arg | G |
| A | ATT - Ile | ACT - Thr | AAT - Asn | AGT - Ser | T |
| | ATC - Ile | ACC - Thr | AAC - Asn | AGC - Ser | C |
| | ATA - Ile | ACA - Thr | AAA - Lys | AGA - Arg | A |
| | ATG - Met | ACG - Thr | AAG - Lys | AGG - Arg | G |
| G | GTT - Val | GCT - Ala | GAT - Asp | GGT - Gly | T |
| | GTC - Val | GCC - Ala | GAC - Asp | GGC - Gly | C |
| | GTA - Val | GCA - Ala | GAA - Glu | GGA - Gly | A |
| | GTG - Val | GCG - Ala | GAG - Glu | GGG - Gly | G |

| One | Three | | |
|---|---|---|---|
| letter | letter | | |
| code | code | Amino acid | Codons |
| A | Ala | Alanine | GCA, GCC, GCG, GCT |
| B | Asx | Asparagine or | AAC, AAT, GAC, GAT |
| | | Aspartic acid | |
| C | Cys | Cysteine | TGC, TGT |
| D | Asp | Aspartic acid | GAC, GAT |
| E | Glu | Glutamic acid | GAA, GAG |
| F | Phe | Phenylalanine | TTC, TTT |
| G | Gly | Glycine | GGA, GGC, GGG, GGT |
| H | His | Histidine | CAC, CAT |
| I | Ile | Isoleucine | ATA, ATC, ATT |
| K | Lys | Lysine | AAA, AAG |
| L | Leu | Leucine | CTA, CTC, CTG, CTT, TTA, TTG |
| M | Met | Methionine | ATG |
| | | (start codon) | |
| N | Asn | Asparagine | AAC, AAT |
| P | Pro | Proline | CCA, CCC, CCG, CCT |
| Q | Gln | Glutamine | CAA, CAG |
| R | Arg | Arginine | AGA, AGG, CGA, CGC, CGG, CGT |
| S | Ser | Serine | AGC, AGT, TCA, TCC, TCG, TCT |
| T | Thr | Threonine | ACA, ACC, ACG, ACT |
| V | Val | Valine | GTA, GTC, GTG, GTT |
| W | Trp | Tryptophan | TGG |
| X | X | any codon | NNN |
| Y | Tyr | Tyrosine | TAC, TAT |
| Z | Glx | Glutamine or | CAA, CAG, GAA, GAG |
| | | Glutamic acid | |
| | * | stop codon (*) | TAA, TAG, TGA |

From a view of process, in some embodiments, the base editing substitutes A in a codon of the VEGFA gene with T, C, or G. In some embodiments, base editing substitutes T in a codon of the VEGFA gene with A, C, or G. In some embodiments, base editing substitutes C in a codon of the VEGFA gene with A, T, or G. In some embodiments, base editing substitutes G in a codon of the VEGFA gene with A, T, or C.

From a view of outcome, in some embodiments, the base editing substitutes a stop codon of the VEGFA gene with a non-stop codon. In some embodiments, base editing substitutes a non-stop codon of the VEGFA gene with a different non-stop codon. In some embodiments, base editing substitutes a non-stop codon of the VEGFA gene with a stop codon.

The VEGFA gene may contain a pathogenic mutation, which may be a pathogenic codon substitution where a wild type codon of the VEGFA gene is substituted with a pathogenic codon. In some embodiments, the wild type codon is a stop codon, and the pathogenic codon is a non-stop codon. The pathogenic codon substitution of a wild type stop codon to a non-stop codon may lead to the expression of a mutant of the expression product of the VEGFA gene containing additional C-terminal amino acids, which mutant may be pathogenic (e.g., non-functional). In some embodiments, the wild type codon is a non-stop codon, and the pathogenic codon is a stop codon or a non-stop codon different from the wild type non-stop codon. The pathogenic codon substitution of a wild type non-stop codon with a stop codon may leads to prematurely terminated (partial) translation of the transcript of the VEGFA gene, which may generate a N-terminal truncation of the expression product of the VEGFA gene or generate no expression product. The N-terminal truncation of the expression product may be pathogenic (e.g., non-functional) and may be stable or unstable (e.g., quickly degraded by cellular enzyme). The pathogenic codon substitution of a wild type non-stop codon with a non-stop codon different from the wild type non-stop codon may lead to the expression of a mutant of the expression product of the VEGFA gene, which mutant may be pathogenic (e.g., non-functional), and which mutant may be stable or unstable (e.g., quickly degraded by cellular enzyme).

In some embodiments, the base editing of the VEGFA gene decreases or eliminates transcription of the VEGFA gene and/or translation of a transcript (e.g., mRNA) of the VEGFA gene. For example, the base editing of the VEGFA gene eliminates full expression of the VEGFA gene by substituting a non-stop codon in the VEGFA gene with a stop codon, leaving no translation or prematurely terminated (partial) translation of the transcript of the VEGFA gene.

In some embodiments, the base editing of the VEGFA gene generates an in-frame stop codon in the VEGFA gene.

In some embodiments, the base editing of the VEGFA gene leads to expression of a functional expression product of the VEGFA gene, e.g., a functional mutant of vascular endothelial growth factor A.

In some embodiments, the base editing of the VEGFA gene leads to expression of a mutant of vascular endothelial growth factor A, for example, by substituting a non-wild type stop codon with a non-wild type, non-stop codon or substituting a non-wild type, non-stop codon with a different non-wild type, non-stop codon. In some embodiments, the mutant of vascular endothelial growth factor A is functional. In some embodiments, the mutant of vascular endothelial growth factor A is non-functional.

In some embodiments, the base editing of the VEGFA gene leads to expression of a non-functional mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the base edited VEGFA gene is a non-functional mutant of vascular endothelial growth factor A.

In some embodiments, the base editing of the VEGFA gene leads to expression of wild type vascular endothelial growth factor A, for example, by substituting a non-wild type stop codon or a non-wild type, non-stop codon with a wild type non-stop codon, and the resulting expression product of the base edited VEGFA gene is wild type vascular endothelial growth factor A.

In some embodiments, the base editing of the VEGFA gene leads to expression of a functional mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the base edited VEGFA gene is a functional mutant of vascular endothelial growth factor A.

In some embodiments, the base editing of the VEGFA gene leads to expression of a non-pathogenic mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the base edited VEGFA gene is non-pathogenic.

In some embodiments, the base editing of the VEGFA gene does not lead to expression of a pathogenic mutant of vascular endothelial growth factor A.

### DNA Prime Editing

Also provided in the disclosure are tools and methods using DNA prime editing to modify the expression of VEGFA gene.

In some embodiments, the napDNAbp is a nickase and further comprises a reverse transcriptase domain, and the guide nucleic acid is a prime editing guide nucleic acid (e.g., pegRNA). As used herein, the term “nickase” means that it substantially lacks dsDNA endonuclease activity (i.e., it is substantially incapable of cleaving both strands of a dsDNA) and is substantially capable of nicking one strand of a dsDNA. In some embodiments, the one strand is the nontarget strand of the dsDNA. In some embodiments, the one strand is the sense strand or the antisense strand of the dsDNA.

In some embodiments, guiding the complex to the VEGFA gene enables the napDNAbp that is a nickase and further comprises a reverse transcriptase domain to specifically prime edit the VEGFA gene in a guide sequence-specific manner.

A general introduction of the prime editing technique may be referred to “Prime Editing: Adding Precision and Flexibility to CRISPR Editing” (blog dot addgene dot org slash prime-editing-crisp-cas-reverse-transcriptase).

Prime editing may allow insertion (addition), deletion, or substitution of one or more nucleotides of the VEGFA gene.

In some embodiments, the prime editing of the VEGFA gene decreases or eliminates transcription of the VEGFA gene and/or translation of a transcript (e.g., mRNA) of the VEGFA gene.

In some embodiments, the prime editing of the VEGFA gene leads to expression of a functional expression product of the VEGFA gene, e.g., a functional mutant of vascular endothelial growth factor A.

In some embodiments, the prime editing of the VEGFA gene generates an in-frame stop codon in the VEGFA gene, for example, by inserting a stop codon, by deleting one or more nucleotides to generate a frameshift mutation that further generates a stop codon, or substituting a non-stop codon with a stop codon.

In some embodiments, the prime editing of the VEGFA gene leads to expression of a mutant of vascular endothelial growth factor A, for example, by substituting a non-wild type stop codon with a non-wild type, non-stop codon or substituting a non-wild type, non-stop codon with a different non-wild type, non-stop codon. In some embodiments, the mutant of vascular endothelial growth factor A is functional. In some embodiments, the mutant of vascular endothelial growth factor A is non-functional.

In some embodiments, the prime editing of the VEGFA gene leads to expression of a non-functional mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the prime edited VEGFA gene is a non-functional mutant of vascular endothelial growth factor A.

In some embodiments, the prime editing of the VEGFA gene leads to expression of wild type vascular endothelial growth factor A, for example, by substituting a non-wild type stop codon or a non-wild type, non-stop codon with a wild type non-stop codon, and the resulting expression product of the prime edited VEGFA gene is wild type vascular endothelial growth factor A.

In some embodiments, the prime editing of the VEGFA gene leads to expression of a functional mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the prime edited VEGFA gene is a functional mutant of vascular endothelial growth factor A.

In some embodiments, the prime editing of the VEGFA gene leads to expression of a non-pathogenic mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the prime edited VEGFA gene is non-pathogenic.

In some embodiments, the prime editing of the VEGFA gene does not lead to expression of a pathogenic mutant of vascular endothelial growth factor A.

### DNA Epigenomic Modification

In some embodiments, the napDNAbp is an endonuclease deficient napDNAbp and further comprises epigenomic modification domain, e.g., methylation domain, demethylation domain. As used herein, the term “endonuclease deficient napDNAbp” is used interchangeably with “endonuclease inactive napDNAbp” or “dead napDNAbp”, meaning that it substantially lacks dsDNA endonuclease activity (i.e., it is substantially incapable of cleaving double strands of a dsDNA), and it substantially lacks nickase activity (i.e., it is substantially incapable of nicking either strand of a dsDNA).

In some embodiments, guiding the complex to the VEGFA gene enables the napDNAbp that is an endonuclease deficient napDNAbp and further comprises epigenomic modification domain, e.g., methylation domain, demethylation domain, to specifically epigenetically modify the VEGFA gene in a guide sequence-specific manner.

In some embodiments, the epigenomic modification of the VEGFA gene decreases or eliminates transcription of the VEGFA gene and/or translation of a transcript (e.g., mRNA) of the VEGFA gene.

In some embodiments, the epigenomic modification of the VEGFA gene increases transcription of the VEGFA gene and/or translation of a transcript (e.g., mRNA) of the VEGFA gene.

### RNA Modification

Provided in the disclosure are multiple ways to modify the expression of VEGFA gene on RNA level. In some embodiments, the transcript of the VEGFA gene is a VEGFA pre-mRNA or a VEGFA mRNA. In some embodiments, the transcript comprises a mutation. In some embodiments, the transcript is a transcript of human VEGFA gene comprising a mutation. In some embodiments, the napBP is a nucleic acid programmable RNA binding protein (napRNAbp).

### RNA Cleavage

Provided in the disclosure are tools and methods using RNA cleavage to modify the expression of VEGFA gene.

In some embodiments, the napRNAbp is a nucleic acid programmable RNA endonuclease (napRNAn).

In some embodiments, guiding the complex to the transcript of the VEGFA gene enables the napRNAn to specifically cleave the transcript of the VEGFA gene in a guide sequence-specific manner. Typically, the cleavage of a transcript by an RNA endonuclease leads to degradation of the transcript.

### RNA Base Editing

Also provided in the disclosure are tools and methods using RNA base editing to modify the expression of VEGFA gene.

In some embodiments, the napRNAbp is an endonuclease deficient napRNAbp and further comprises a base editing domain. As used herein, the term “endonuclease deficient napRNAbp” is used interchangeably with “endonuclease inactive napRNAbp” or “dead napRNAbp”, meaning that it substantially lacks RNA endonuclease activity (i.e., it is substantially incapable of cleaving an RNA).

In some embodiments, guiding the complex to the transcript of the VEGFA gene enables the napRNAbp that is an endonuclease deficient napRNAbp and further comprises the base editing domain to specifically base edit the transcript in a guide sequence-specific manner.

In some embodiments, the base editing of the transcript substitutes a nucleotide of the transcript to a different nucleotide. In some embodiments, the base editing of the transcript substitutes one nucleotide of the transcript to one different nucleotide, which is also known as single base editing. In some embodiments, the base editing of the transcript substitutes two or three or more nucleotides, whether consecutive or not, of the transcript to two or three or more different nucleotides, which is also known as multiple or multiplexed base editing.

From a view of process, in some embodiments, the base editing substitutes A in a codon of the transcript with U, C, or G; substitutes U in a codon of the transcript with A, C, or G; substitutes C in a codon of the transcript with A, U, or G; or substitutes G in a codon of the transcript with A, U, or C.

From a view of outcome, in some embodiments, the base editing substitutes a stop codon of the transcript with a non-stop codon; substitutes a non-stop codon of the transcript with a different non-stop codon; or substitutes a non-stop codon of the transcript with a stop codon.

The transcript may contain a pathogenic mutation, which may be a pathogenic codon substitution where a wild type codon of the transcript is substituted with a pathogenic codon. In some embodiments, the wild type codon is a stop codon, and the pathogenic codon is a non-stop codon. The pathogenic codon substitution of a wild type stop codon to a non-stop codon may lead to the translation of a mutant of the expression product of the VEGFA gene containing additional C-terminal amino acids, which mutant may be pathogenic (e.g., non-functional). In some embodiments, the wild type codon is a non-stop codon, and the pathogenic codon is a stop codon or a non-stop codon different from the wild type non-stop codon. The pathogenic codon substitution of a wild type non-stop codon with a stop codon may leads to prematurely terminated (partial) translation of the transcript, which may generate a N-terminal truncation of the expression product of the VEGFA gene or generate no expression product. The N-terminal truncation of the expression product may be pathogenic (e.g., non-functional) and may be stable or unstable (e.g., quickly degraded by cellular enzyme). The pathogenic codon substitution of a wild type non-stop codon with a non-stop codon different from the wild type non-stop codon may lead to the translation of a mutant of the expression product of the VEGFA gene, which mutant may be pathogenic (e.g., non-functional), and which mutant may be stable or unstable (e.g., quickly degraded by cellular enzyme).

In some embodiments, the base editing of the transcript decreases or eliminates translation of the transcript. For example, the base editing of the transcript eliminates full translation of the transcript by substituting a non-stop codon in the transcript with a stop codon, leaving no translation or prematurely terminated (partial) translation of the transcript.

In some embodiments, the base editing of the transcript generates an in-frame stop codon in the transcript.

In some embodiments, the base editing of the transcript leads to translation of a functional expression product of the transcript, e.g., a functional mutant of vascular endothelial growth factor A.

In some embodiments, the base editing of the transcript leads to expression of a mutant of vascular endothelial growth factor A, for example, by substituting a non-wild type stop codon with a non-wild type, non-stop codon or substituting a non-wild type, non-stop codon with a different non-wild type, non-stop codon. In some embodiments, the mutant of vascular endothelial growth factor A is functional. In some embodiments, the mutant of vascular endothelial growth factor A is non-functional.

In some embodiments, the base editing of the transcript leads to expression of a non-functional mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the transcript is a non-functional mutant of vascular endothelial growth factor A.

In some embodiments, the base editing of the transcript leads to expression of wild type vascular endothelial growth factor A, for example, by substituting a non-wild type stop codon or a non-wild type, non-stop codon with a wild type non-stop codon, and the resulting expression product of the transcript is wild type vascular endothelial growth factor A.

In some embodiments, the base editing of the transcript leads to expression of a functional mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the transcript is a functional mutant of vascular endothelial growth factor A.

In some embodiments, the base editing of the transcript leads to expression of a non-pathogenic mutant of vascular endothelial growth factor A, for example, by substituting a non-wild type stop codon with a non-wild type, non-stop codon or substituting a non-wild type, non-stop codon with a different non-wild type, non-stop codon, and the resulting expression product of the transcript is non-pathogenic.

In some embodiments, the base editing of the transcript gene does not lead to expression of a pathogenic mutant of vascular endothelial growth factor A.

napBP

For the purpose of the disclosure, the napBP is capable of forming a complex with the guide nucleic acid of the disclosure by complexing with the scaffold sequence of the guide nucleic acid and is thereby guided to the VEGFA gene or transcript thereof via the hybridization of the guide sequence of the guide nucleic acid to the target sequence of the VEGFA gene or transcript. When the napBP is guided to the VEGFA gene or transcript, the activity of the napBP (or the functional domain associated with (e.g., bound to) the napBP) functions to modify the VEGFA gene or transcript thereof, leading to modification of expression of the VEGFA gene. Generally, any such napBP can be used with the guide nucleic acid of the disclosure, and when a napBP is selected, the scaffold sequence compatible to the napBP for complexing with the napBP can also be selected accordingly. The scaffold sequence is generally conserved.

In some embodiments, the napBP (e.g., napDNAbp) is capable of recognizing a protospacer adjacent motif (PAM) on the nontarget strand of the VEGFA gene, wherein the PAM is immediately 5′ or 3′ to a protospacer sequence on the nontarget strand of the VEGFA gene, and wherein the protospacer sequence is fully reversely complementary to the target sequence.

In some embodiments, the PAM comprises sequence 5′-NN-3′, 5′-NNN-3′, 5′-NNNN-3′, 5′-NNNNN-3′, or 5′-NNNNNN-3′, wherein N is A, T, G, or C.

Non-limiting examples of the napBP include CRISPR-associated (Cas) protein, IscB, TAL nuclease, meganuclease, and zinc-finger nuclease. Non-limiting examples of CRISPR-associated (Cas) protein include Cas9 (e.g., dCas9 and nCas9), Cas12a/Cpf1, Cas12b/C2c1, Cas12c/C2c3, Cas12d/CasY, Cas12e/CasX, Cas12f/Cas14, Cas12g, Cas12h, Cas12i, and Cas12k. Non-limiting examples of Cas protein include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12f/Cas14, Cas12g, Cas12h, Cas12i, Cas12k, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, IscB, homologues thereof, or modified or engineered versions thereof. Other napDNAbp are also within the scope of this disclosure, e.g., IscB, IsrB, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference.

In some embodiments, the Cas protein is an endonuclease, a nickase, or a dead Cas.

In some embodiments, the PAM comprises sequence 5′-NTN-3′, wherein N is A, T, G, or C, and wherein the PAM is immediately 5′ to the protospacer sequence. In some embodiments, the PAM comprises sequence 5′-TTN-3′, wherein N is A, T, G, or C. For example, in some embodiments, the napBP is a Class 2, Type V CRISPR-associated protein (Cas12). In some embodiments, the Cas12 is Cas12a (Cpf1), Cas12b (C2c1), Cas12c (C2c3), Cas12d (CasY), Cas12e (CasX), Cas12f (Cas14), Cas12i, or Cas12k (C2c10, C2C7), e.g., Cas12i1, Cas12i1, Cas12i3, Cas12i4, xCas12i (SiCas12i), Cas12Max, hfCas12Max, or a mutant thereof.

In some embodiments, the PAM comprises sequence 5′-NGG-3′, wherein N is A, T, G, or C, and wherein the PAM is immediately 3′ to the protospacer sequence. For example, in some embodiments, the napBP is a Class 2, Type II CRISPR-associated protein (Cas9), e.g., SaCas9, SpCas9, or a mutant thereof.

In some embodiments, the PAM comprises sequence 5′-NNNGAN-3′, wherein N is A, T, G, or C, and wherein the PAM is immediately 3′ to the protospacer sequence. For example, in some embodiments, the napBP is a IscB protein, e.g., OgeuIscB or a mutant thereof.

In some cases, the recognizing ability of the napBP to a target nucleic acid may not be limited to any specific PAM, which means that the napBP can recognize any PAM, such that the PAM is not a substantial restriction on the selection of a protospacer sequence or a target sequence. Such a napBP is called “PAMless”, for example, a PAMless SpCas9 mutant. In some embodiments, the napBP is PAMless.

In some embodiments, the napBP is a Class 2, Type VI CRISPR-associated protein (Cas13). Cas13 can be targeted to RNA by a guide nucleic acid. Cas13 is particularly useful since there is no PAM restriction for eukaryotic transcripts when Cas13 is used as the napBP. In some embodiments, the Cas13 is Cas13a (C2c2), Cas13b (such as, Cas13b1, Cas13b2), Cas13c, Cas13d, Cas13e (Cas13X), Cas13f (Cas13Y), or a mutant thereof. In some embodiments. The napBP is hfCas13Y (hfCas13f/hfCas13f.1) in PCT/CN2022/122833. As used herein, the term “Cas13Y” is used interchangeably with “Cas13f”, and the term “hfCas13Y” is used interchangeably with “hfCas13f” and “hfCas13f.1”.

Typically, a Cas protein (e.g., Cas9, Cas12, Cas13) can associate with a CRISPR RNA (crRNA) comprising a spacer sequence that guides the Cas protein to a specific sequence that is reversely complementary and capable of hybridizing to the spacer sequence of the crRNA. The crRNA also comprises a scaffold sequence capable of complexing with the Cas protein.

In some embodiments, the napBP comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 16 or a N-terminal truncation thereof without the first N-terminal Methionine (SEQ ID NO: 32). In some embodiments, the napBP retains at least 80% (e.g., at least about 80%, 85%, 90%, 91%, 92%, 93%, 94%, 95%, 96%, 97%, 98%, 99%, or 100%) of the guide sequence-specific DNA endonuclease activity of the sequence of SEQ ID NO: 16 or 32.

In some embodiments, the napBP comprises a sequence of SEQ ID NO: 16 or 32.

In some embodiments, the sequence encoding the napBP comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 9 or a 5′ end truncation thereof without the first 5′ ATG codon (SEQ ID NO: 33). In some embodiments, the napBP encoded by the sequence retains at least 80% (e.g., at least about 80%, 85%, 90%, 91%, 92%, 93%, 94%, 95%, 96%, 97%, 98%, 99%, or 100%) of the guide sequence-specific DNA endonuclease activity of the sequence of SEQ ID NO: 9 or 33.

In some embodiments, the sequence encoding the napBP comprises a sequence of SEQ ID NO: 9 or 33.

### Base Editing

In some embodiments, the napBP further comprises a base editing domain.

In some embodiments, the base editing domain is capable of substituting a base of a nucleotide with a different base.

In some embodiments, the base editing domain is capable of deaminating a base of a nucleotide.

In some embodiments, the base editing domain comprises a deaminase domain capable of deaminating a base (e.g., an adenine, a guanine, a cytosine, a thymine, an uracil) of a nucleotide. In some embodiments, the deaminase domain is capable of deaminating an adenine (A) to a hypoxanthine (I). In some embodiments, the deamination of the adenine to the hypoxanthine converts the adenosine (A) or deoxyadenosine (dA) containing the adenine to a guanosine (G) or deoxyguanosine (dG). In some embodiments, the deaminase domain is capable of deaminating a cytosine (C) to an uracil (U). In some embodiments, the deamination of the cytosine to the uracil converts the cytidine (C) or deoxycytidine (dC) containing the cytosine to a uridine (U) or a deoxythymidine (dT).

In some embodiments, the base editing domain is capable of excising a base (e.g., an adenine, a guanine, a cytosine, a thymine, an uracil) of a nucleotide.

In some embodiments, the base editing domain comprises a base excising domain capable of excising a base of a nucleotide.

In some embodiments, the base editing domain comprises a deaminase domain and a base excising domain.

In some embodiments, the deaminase domain is a double-stranded RNA-specific adenosine deaminase (ADAR), such as, ADAR1, ADAR2 (ADARB1), ADAR3 (ADARB2), or the deaminase domain thereof, or a mutant thereof.

In some embodiments, the ADAR is human ADAR or ADAR of non-human species. In some embodiments, the ADAR is octopus ADAR.

In some embodiments, the deaminase domain is human ADAR2 deaminase domain (hADAR2DD).

In some embodiments, the deaminase domain is a mutant of hADAR2DD, e.g., hADAR2DD−E488Q, hADAR2DD−E488Q+T375G, hADAR2DD−E488Q+V351G+S486A+T375S+S370C+P462A+N597I+L332I+I398V+K350I+M383L+D619G+S582T+V440I+S495N+K418E+S661T, hADAR2DD−E488Q+V351G+S486A+T375A+S370C+P462A+N597I+L332I+I398V+K350I+M383L+D619G+S582T+V440I+S495N+K418E+S661T.

In some embodiments, the deaminase domain is apolipoprotein B mRNA editing enzyme, catalytic polypeptide (APOBEC), such as, APOBEC1, APOBEC2, APOBEC3A, APOBEC3B, APOBEC3C, APOBEC3D, APOBEC3E, APOBEC3F, APOBEC3G, APOBEC3H, APOBEC4, or the deaminase domain thereof, or a mutant thereof.

In some embodiments, the deaminase domain is activation-induced cytidine deaminase (AID, AICDA, single-stranded DNA cytosine deaminase), or the deaminase domain thereof, or a mutant thereof.

In some embodiments, the deaminase domain is tRNA adenosine deaminase (TadA), or the deaminase domain thereof, or a mutant thereof.

### Epigenomic Modification

In some embodiments, the napBP further comprises a transcription inhibiting domain (e.g., KRAB domain or SID domain). In some embodiments, the napBP further comprises a KRAB domain.

In some embodiments, the napBP further comprises a DNA methyltransferase, such as, DNMT31, DNMT3a.

In some embodiments, the napBP further comprises a DNMT31 domain and a DNMT3a domain.

In some embodiments, the napBP further comprises a DNMT31 domain, a DNMT3a domain, and a KRAB domain.

In some embodiments, the napBP, the KRAB domain, the DNMT31 domain, and the DNMT3a domain are arranged from N-terminal to C-terminal.

### Design of Protospacer Sequence/Target Sequence; Target Site

For the purpose of the disclosure, in some embodiments, the protospacer sequence or target sequence is located such that the VEGFA gene or transcript thereof can be specifically modified by the napBP or a functional domain associated with the napBP.

To facilitate the evaluation of selected protospacer sequences or target sequence and designed guide sequences in mouse models, in some embodiments, the protospacer sequence or target sequence is located such that a mouse VEGFA gene or transcript thereof can be specifically modified by the napBP or a functional domain associated with the napBP. In some embodiments, the protospacer sequence or target sequence is located such that both a human VEGFA gene or transcript thereof and a mouse VEGFA gene or transcript thereof can be specifically modified by the napBP or a functional domain associated with the napBP. That is, the protospacer sequence or target sequence is selected to be cross-reactive to both human and mouse species.

In some embodiments, the protospacer sequence is a stretch of contiguous nucleotides identified from the nontarget strand of the VEGFA gene by identifying the stretch of contiguous nucleotides immediately 5′ or 3′, and optionally, 3′, to the PAM on the nontarget strand.

In some embodiments, the protospacer sequence is a stretch of about, at least about, or at most about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the nontarget strand of the VEGFA gene, or a stretch of contiguous nucleotides on the nontarget strand of the VEGFA gene in a numerical range between any two of the preceding values, e.g., a stretch of from about 16 to about 50 contiguous nucleotides. In some embodiments, the protospacer sequence is a stretch of about 20, 30, or 50 contiguous nucleotides on the nontarget strand of the VEGFA gene.

In some embodiments, the protospacer sequence on the nontarget strand of the VEGFA gene corresponding to the target sequence is immediately 3′ to PAM of 5′-NTN-3′, wherein N is A, T, G, or C.

In some embodiments, the protospacer sequence on the nontarget strand of the VEGFA gene corresponding to the target sequence is immediately 5′ to PAM of 5′-NGG-3′, wherein N is A, T, G, or C.

In some embodiments, the protospacer sequence on the nontarget strand of the VEGFA gene corresponding to the target sequence is immediately 5′ to PAM of 5′-NNNGAN-3′, wherein N is A, T, G, or C.

In some embodiments, the protospacer sequence is a stretch of about 20, 30, or 50 contiguous nucleotides of the nontarget strand of the VEGFA gene immediately 3′ to PAM of 5′-NTN-3′, wherein N is A, T, G, or C.

In some embodiments, the protospacer sequence is a stretch of about 20, 30, or 50 contiguous nucleotides of the nontarget strand of the VEGFA gene immediately 5′ to PAM of 5′-NGG-3′, wherein N is A, T, G, or C.

In some embodiments, the protospacer sequence is a stretch of about 20, 30, or 50 contiguous nucleotides of the nontarget strand of the VEGFA gene immediately 5′ to PAM of 5′-NNNGAN-3′, wherein N is A, T, G, or C.

In some embodiments, the target sequence is a stretch of contiguous nucleotides identified from the target strand of the VEGFA gene or from the transcript thereof.

In some embodiments, the target sequence is a stretch of about, at least about, or at most about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the target strand of the VEGFA gene or on the transcript thereof, or a stretch of contiguous nucleotides on the target strand of the VEGFA gene or on the transcript thereof in a numerical range between any two of the preceding values, e.g., a stretch of from about 16 to about 50 contiguous nucleotides. In some embodiments, the target sequence is a stretch of about 20, 30, or 50 contiguous nucleotides on the target strand of the VEGFA gene or on the transcript thereof.

In some embodiments, the nontarget strand is the sense strand of the VEGFA gene.

In some embodiments, the nontarget strand is the antisense strand of the VEGFA gene.

In some embodiments, the target strand is the sense strand of the VEGFA gene.

In some embodiments, the target strand is the antisense strand of the VEGFA gene.

In some embodiments, the protospacer sequence on the nontarget strand of the VEGFA gene corresponding to the target sequence or target sequence on the target strand of the VEGFA gene is located at or within an exon of the VEGFA gene or transcript thereof, or at or within a splice donor or a splice acceptor of the exon.

In some embodiments, the exon is selected from the group consisting of Exon 1, Exon 2, Exon 3, Exon 4, Exon 5, Exon 6, Exon 7, and Exon 8.

In some embodiments, the VEGFA gene is human VEGFA gene, non-human primate VEGFA gene, or mouse VEGFA gene. In some embodiments, the VEGFA mRNA is human VEGFA mRNA, non-human primate VEGFA mRNA, or mouse VEGFA mRNA.

In some embodiments, the VEGFA gene or transcript thereof is in a eukaryotic cell, for example, a human cell, a non-human primate cell, or a mouse cell. In some embodiments, the VEGFA gene or transcript thereof is in a retinal cell.

### Design of Guide Sequence According to Protospacer/Target Sequence

In some embodiments, the guide sequence is in a length of about, at least about, or at most about 14 nucleotides, e.g., about, at least about, or at most about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides, or in a length of nucleotides in a numerical range between any two of the preceding values, e.g., in a length of from about 16 to about 50 nucleotides. In some embodiments, the guide sequence is in a length of about 20, 30, or 50 nucleotides.

In some embodiments, (1) the guide sequence is at least about 50%, 55%, 60%, 65%, 70%, 75%, 80%, 85%, 90%, 95%, or 100% (fully), optionally about 100% (fully), reversely complementary to the target sequence; (2) the guide sequence contains no more than 5, 4, 3, 2, or 1 mismatch or contains no mismatch with the target sequence; or (3) the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence when the PAM is immediately 5′ to the protospacer sequence or at the 3′ end of the guide sequence when the PAM is immediately 3′ to the protospacer sequence. In some embodiments, the guide sequence is about 100% (fully), reversely complementary to the target sequence.

In some embodiments, the guide sequence contains 1 mismatch with the target sequence. In some embodiments, the guide sequence is about 98% reversely complementary to the target sequence. In some embodiments, the 1 mismatch in the guide sequence is at a position corresponding the nucleotide of the target sequence that is intended to be substituted.

### Selection of Protospacer/Target/Guide Sequence; Effect of System

In some embodiments, the protospacer sequence, the target sequence, or the guide sequence is selected according to the knockdown efficiency of the napRNAbp guided to the VEGFA transcript by the guide sequence. The knockdown efficiency can be represented by, for example, fold change of VEGFA transcript or protein measured by RT-qPCR or western blot. The higher the knockdown efficiency is, the more suitable the protospacer sequence, the target sequence, or the guide sequence is.

In some embodiments, the protospacer sequence, the target sequence, or the guide sequence is selected according to the level of transcription of the VEGFA gene and/or level of translation of a transcript (e.g., mRNA) of the VEGFA gene (level of expression of the VEGFA gene) in in vivo animal model. The lower the level is, the more suitable the protospacer sequence, the target sequence, or the guide sequence is.

In some embodiments, the level of the transcript (e.g., mRNA) of the VEGFA gene is decreased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell model or the animal model, compared to the level of the transcript (e.g., mRNA) of the VEGFA gene in the same cell model or animal model that does not receive the administration.

In some embodiments, the level of the expression product (e.g., vascular endothelial growth factor A) of the VEGFA gene is decreased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell or the animal model, compared to the level of the expression product (e.g., vascular endothelial growth factor A) of the VEGFA gene in the same cell model or animal model that does not receive the administration.

### Overall Structure of Guide Nucleic Acid

In some embodiments, the guide nucleic acid comprises a scaffold sequence 5′ to a guide sequence. In some embodiments, the guide nucleic acid comprises a scaffold sequence 3′ to a guide sequence.

In some embodiments, the guide nucleic acid comprises one scaffold sequence and one guide sequence.

In some embodiments, the guide nucleic acid comprises one scaffold sequence 5′ to one guide sequence. In some embodiments, the guide nucleic acid comprises one scaffold sequence 3′ to one guide sequence.

In some embodiments, the guide nucleic acid comprises one or more scaffold sequence and/or one or more guide sequence, provided that the guide nucleic acid does not comprise one scaffold sequence and one guide sequence.

In some embodiments, the guide nucleic acid comprises, from 5′ to 3′, one scaffold sequence, one guide sequence, and one scaffold sequence, wherein scaffold sequences are the same or different.

In some embodiments, the guide nucleic acid comprises, from 5′ to 3′, one guide sequence, one scaffold sequence, and one guide sequence, wherein guide sequences are the same or different.

In some embodiments, the guide nucleic acid comprises, from 5′ to 3′, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one guide sequence, one scaffold sequence, one guide sequence, and one scaffold sequence, wherein scaffold sequences are the same or different, and wherein guide sequences are the same or different.

In some embodiments, the guide nucleic acid comprises, from 5′ to 3′, one scaffold sequence, one guide sequence, one scaffold sequence, one guide sequence, and one scaffold sequence, wherein scaffold sequences are the same or different, and wherein guide sequences are the same or different.

In some embodiments, the guide nucleic acid comprises, from 5′ to 3′, one guide sequence, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one scaffold sequence, one guide sequence, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one guide sequence, one scaffold sequence, one guide sequence, one scaffold sequence, one guide sequence, and one scaffold sequence, wherein scaffold sequences are the same or different, and wherein guide sequences are the same or different.

In some embodiments, the guide nucleic acid comprises a linker or no linker between any adjacent scaffold sequence and guide sequence. In some embodiments, the guide nucleic acid comprises no linker between any adjacent scaffold sequence and guide sequence.

### Multiple Guide Nucleic Acid

The system or rAAV vector genome of the disclosure may comprise or encode one guide nucleic acid or comprise or encode multiple (e.g., 2, 3, 4, or more) guide nucleic acids, e.g., for the purpose of improving the editing efficiency of the system.

In some embodiments, the system further comprises one or more additional guide nucleic acids, or the first polynucleotide sequence further comprises one or more additional sequences encoding one or more additional guide nucleic acids, each of the additional guide nucleic acids comprising:
(1) an additional scaffold sequence capable of forming a complex with the napBP, and
(2) an additional guide sequence capable of hybridizing to an additional target sequence on a target strand of the VEGFA gene or an additional target sequence on the transcript thereof, thereby guiding the complex to the VEGFA gene or the transcript.

In some embodiments, the additional protospacer sequence is on the same strand as the protospacer sequence.

In some embodiments, the additional protospacer sequence is on the different strand from the protospacer sequence.

In some embodiments, the additional protospacer sequence is the same or different from the protospacer sequence.

In some embodiments, the additional target sequence is the same or different from the target sequence.

In some embodiments, the additional guide sequence is the same or different from the guide sequence.

In some embodiments, the additional scaffold sequence is the same or different from the scaffold sequence. In some embodiments wherein the system comprises the same napBP and multiple guide nucleic acids, the scaffold sequences of the multiple guide nucleic acids may be the same or different (e.g., different by no more than 5, 4, 3, 2, or 1 nucleotide) to be compatible to the same napBP. In some embodiments wherein that the system comprises different napBP (e.g., a Cas12i and a Cas9) and multiple guide nucleic acids, the scaffold sequences of the multiple guide nucleic acids may be different to be compatible to the different napBP, respectively.

In some embodiments, the additional guide nucleic acid and the guide nucleic acid are operably linked to or under the regulation of the same regulatory element (e.g., promoter) or separate regulatory elements (e.g., separate promoters).

### Nature and Modification of Guide Nucleic Acid

In some embodiments, the guide nucleic acid (e.g., the guide nucleic acid, the additional guide nucleic acid) is an RNA, i.e., a guide RNA (gRNA). In some embodiments, the guide nucleic acid is an unmodified guide RNA. In some embodiments, the guide nucleic acid is a modified guide RNA. In some embodiments, the guide nucleic acid comprises a modification. In some embodiments, the guide nucleic acid is a modified RNA containing a modified ribonucleotide. In some embodiments, the guide nucleic acid is a modified RNA containing a deoxyribonucleotide. In some embodiments, the guide nucleic acid is a modified RNA containing a modified deoxyribonucleotide. In some embodiments, the guide nucleic acid comprises a modified or unmodified deoxyribonucleotide and a modified or unmodified ribonucleotide.

### Specific Target and Guide Sequences

In some embodiments, the guide sequence or the additional guide sequence comprises (1) a sequence of SEQ ID NO: 1 or 2 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 1 or 2 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 1 or 2.

In some embodiments, the guide sequence or the additional guide sequence comprises a sequence of SEQ ID NO: 1 or 2.

In some embodiments, the target sequence or the additional target sequence comprises (1) a sequence of SEQ ID NO: 18 or 19 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 18 or 19 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 18 or 19.

### Specific Scaffold Sequence

For the purpose of the disclosure, the scaffold sequence is compatible with the napBP of the disclosure and is capable of complexing with the napBP. The scaffold sequence may be a naturally occurring scaffold sequence identified along with the napBP, or a variant thereof maintaining the ability to complex with the napBP. Generally, the ability to complex with the napBP is maintained as long as the secondary structure of the variant is substantially identical to the secondary structure of the naturally occurring scaffold sequence. A nucleotide deletion, insertion, or substitution in the primary sequence of the scaffold sequence may not necessarily change the secondary structure of the scaffold sequence (e.g., the relative locations and/or sizes of the stems, bulges, and loops of the scaffold sequence do not significantly deviate from that of the original stems, bulges, and loops). For example, the nucleotide deletion, insertion, or substitution may be in a bulge or loop region of the scaffold sequence so that the overall symmetry of the bulge and hence the secondary structure remains largely the same. The nucleotide deletion, insertion, or substitution may also be in the stems of the scaffold sequence so that the lengths of the stems do not significantly deviate from that of the original stems (e.g., adding or deleting one base pair in each of two stems correspond to 4 total base changes).

In some embodiments, the scaffold sequence or the additional scaffold sequence has substantially the same secondary structure as the secondary structure of the sequence of SEQ ID NO: 5.

In some embodiments, the scaffold sequence or the additional scaffold sequence comprises (1) a sequence of SEQ ID NO: 5 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 5 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 5.

In some embodiments, the scaffold sequence or the additional scaffold sequence comprises the sequence of SEQ ID NO: 5.

### Specific Guide Nucleic Acid Sequence

In some embodiments, the guide nucleic acid comprises a sequence having a sequence identity of at least about 80% (e.g., at least about 80%, 85%, 90%, 91%, 92%, 93%, 94%, 95%, 96%, 97%, 98%, 99%, or 100%) to the sequence of any one of SEQ ID NOs: 17 and 30-31; or a sequence having at most 1, 2, 3, 4, 5, 6, 7, 8, 9, or 10 nucleotide differences, whether consecutive or not, compared to the sequence of any one of SEQ ID NOs: 17 and 30-31.

In some embodiments, the guide nucleic acid comprises a sequence of any one of SEQ ID NOs: 17 and 30-31.

### Regulation of Guide Nucleic Acid

Also provided in the disclosure is a polynucleotide comprising or encoding the guide nucleic acid.

In some embodiments, the polynucleotide comprising or encoding the guide nucleic acid is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the guide nucleic acid is operably linked to or under the regulation of a promoter.

In some embodiments, the first polynucleotide sequence comprises a promoter operably linked to the sequence encoding the guide nucleic acid.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

In some embodiments, the promoter is a U6 promoter.

In some embodiments, the promoter comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 4 or 22; or a sequence having at most 1, 2, 3, 4, 5, 6, 7, 8, 9, or 10 nucleotide differences, whether consecutive or not, compared to the sequence of SEQ ID NO: 4 or 22.

In some embodiments, the first polynucleotide sequence comprises, from 5′ to 3′, a promoter of SEQ ID NO: 4, and a first sequence encoding the guide nucleic acid of any one of SEQ ID NOs: 17 and 30-31.

Regulation of napBP

In some embodiments, the polynucleotide encoding the napBP is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the polynucleotide encoding the napBP is operably linked to or under the regulation of a promoter.

In some embodiments, the second polynucleotide sequence comprises a promoter operably linked to the sequence encoding the napBP.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (le) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a human synapsin (hSyn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a VEGFA promoter, a GRK1 promoter, a CRX promoter, a NRL promoter, a MECP2 promoter, a mMECP2 promoter, a hMECP2 promoter, an APP promoter, and a RCVRN promoter.

In some embodiments, the promoter is an eye or retina specific promoter.

In some embodiments, the promoter is a Cbh promoter.

In some embodiments, the second polynucleotide sequence comprises a Kozak sequence (gccacc) 5′ to the sequence encoding the napBP.

In some embodiments, the second polynucleotide sequence comprises a sequence encoding a nuclear localization signal (NLS) 5′ and/or 3′ to the sequence encoding the napBP.

In some embodiments, the second polynucleotide sequence comprises a sequence encoding a nuclear export signal (NES) 5′ and/or 3′ to the sequence encoding the napBP.

In some embodiments, the second polynucleotide sequence comprises a sequence encoding a first NLS 5′ to the sequence encoding the napBP and a second sequence encoding a second NLS 3′ to the sequence encoding the napBP.

In some embodiments, the NLS, the first NLS, and/or the second NLS is a SV40 NLS (SEQ ID NO: 26), a bpSV40 NLS (SEQ ID NO: 15), or a Nucleoplasmin NLS (npNLS).

In some embodiments, the napBP comprises a bpSV40 NLS at the N-terminal of the napBP and a bpSV40 NLS at the C-terminal of the napBP.

In some embodiments, the napBP comprises a NLS inserted between the N-terminal Met and the remaining amino acid sequence of the napBP.

In some embodiments, the second polynucleotide comprises a sequence encoding a first NLS at the 5′ end of the sequence encoding the Cas13 endonuclease.

In some embodiments, the second polynucleotide comprises a sequence encoding a first NLS inserted between the 5′ start codon ATG and the remaining polynucleotide sequence of the sequence encoding the Cas13 endonuclease.

In some embodiments, the second polynucleotide comprises a sequence encoding a second NLS at the 3′ end of the sequence encoding the Cas13 endonuclease.

In some embodiments, the second polynucleotide sequence comprises a WPRE sequence downstream of the sequence encoding the napBP.

In some embodiments, the WPRE sequence is selected from the group consisting of Woodchuck Hepatitis Virus (WHP) Posttranscriptional Regulatory Element (WPRE), WPRE3 (a shortened WPRE), and a functional variant (e.g., a functional truncation) thereof.

In some embodiments, the second polynucleotide sequence comprises a sequence encoding a polyadenylation (polyA) signal downstream of the sequence encoding the napBP.

In some embodiments, the second polynucleotide sequence comprises, downstream of the sequence encoding the napBP, a WPRE sequence followed by a sequence encoding a polyadenylation (polyA) signal.

In some embodiments, the polyA signal is selected from a group consisting of a bovine growth hormone polyadenylation (bGH polyA) signal, a small polyA (SPA) signal, a human growth hormone polyadenylation (hGH polyA) signal, a SV40 polyA (SV40 polyA) signal, a rabbit beta globin polyA (rBG polyA) signal, a combination of SV40 late polyadenylation signal upstream element and SV40 late polyadenylation signal, and a functional variant (e.g., a functional truncation) thereof.

In some embodiments, the polyA signal is a bGH polyA signal.

In some embodiments, the second polynucleotide sequence comprises, from 5′ to 3′, the promoter, the Kozak sequence, an in-frame start codon ATG, the first sequence encoding the first NLS, the sequence encoding the napBP, the second sequence encoding the second NLS, an in-frame stop codon, the WPRE sequence, and the sequence encoding the polyA signal.

In some embodiments, the promoter comprises a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 6; or a sequence having at most 1, 2, 3, 4, 5, 6, 7, 8, 9, or 10 nucleotide differences, whether consecutive or not, compared to the sequence of SEQ ID NO: 6.

In some embodiments, the Kozak sequence is gccacc.

In some embodiments, the NLS, the first NLS, and/or the second NLS comprises a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 15; or a sequence having at most 1, 2, 3, 4, 5, 6, 7, 8, 9, or 10 amino acid differences, whether consecutive or not, compared to the sequence of SEQ ID NO: 15.

In some embodiments, the sequence encoding the NLS, the first sequence encoding the first NLS, and/or the second sequence encoding the second NLS comprises a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 8 or 10; or a sequence having at most 1, 2, 3, 4, 5, 6, 7, 8, 9, or 10 nucleotide differences, whether consecutive or not, compared to the sequence of SEQ ID NO: 8 or 10.

In some embodiments, the WPRE sequence comprises a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 11; or a sequence having at most 1, 2, 3, 4, 5, 6, 7, 8, 9, or 10 nucleotide differences, whether consecutive or not, compared to the sequence of SEQ ID NO: 11.

In some embodiments, the sequence encoding the polyA signal comprises a sequence encoding a polypeptide having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 12; or a sequence having at most 1, 2, 3, 4, 5, 6, 7, 8, 9, or 10 nucleotide differences, whether consecutive or not, compared to the sequence of SEQ ID NO: 12.

In some embodiments, the second polynucleotide sequence comprises, from 5′ to 3′, the promoter of SEQ ID NO: 6, Kozak sequence of gccacc, an in-frame start codon ATG, the first sequence encoding the first NLS of SEQ ID NO: 15, the sequence encoding the napBP of SEQ ID NO: 16 or 32, the second sequence encoding the second NLS of SEQ ID NO: 15, an in-frame stop codon, the WPRE sequence of SEQ ID NO: 11, and the sequence encoding the polyA signal of SEQ ID NO: 12.

In some embodiments, the second polynucleotide sequence comprises, from 5′ to 3′, the promoter of SEQ ID NO: 6, Kozak sequence of gccacc, an in-frame start codon ATG, the first sequence of SEQ ID NO: 8 or 10 encoding the first NLS, the sequence of SEQ ID NO: 9 or 33 encoding the napBP, the second sequence of SEQ ID NO: 8 or 10 encoding the second NLS, an in-frame stop codon, the WPRE sequence of SEQ ID NO: 11, and the sequence encoding the polyA signal of SEQ ID NO: 12.

Full Length rAAV Vector Genome

In some embodiments, the rAAV vector genome comprises a 5′ inverted terminal repeat (ITR) sequence and a 3′ ITR sequence.

In some embodiments, the 5′ ITR sequence and the 3′ ITR sequence are both wild-type ITR sequences from AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV PHP.eB, or a member of the Clade to which any of the AAV1-AAV13 belong, or a functional variant (e.g., a functional truncation) thereof.

In some embodiments, the 5′ ITR sequence comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 3 or 28.

In some embodiments, the 3′ ITR sequence comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 13 or 29.

In some embodiments, the rAAV vector genome comprises, from 5′ to 3′, the first polynucleotide sequence and the second polynucleotide sequence.

In some embodiments, the rAAV vector genome comprises, from 5′ to 3′, the second polynucleotide sequence and the first polynucleotide sequence.

In some embodiments, the rAAV vector genome comprises, from the 5′ to 3′,
(1) a 5′ ITR as described in the disclosure;
(2) a promoter as described in the disclosure operably linked to a sequence encoding a guide nucleic acid;
(3) a sequence encoding a guide nucleic acid as described in the disclosure;
(4) a promoter as described in the disclosure operably linked to a sequence encoding a napBP;
(5) optionally, a Kozak sequence as described in the disclosure;
(6) an in-frame start codon ATG;
(7) optionally, a first sequence encoding a first NLS as described in the disclosure;
(8) a sequence encoding the napBP as described in the disclosure;
(9) optionally, a second sequence encoding a second NLS as described in the disclosure;
(10) an in-frame stop codon,
(11) optionally, a WPRE sequence as described in the disclosure;
(12) a sequence encoding a polyA signal as described in the disclosure; and
(13) a 3′ ITR as described in the disclosure.

In some embodiments, the rAAV vector genome comprises, from the 5′ to 3′,
(1) the 5′ ITR of SEQ ID NO: 3;
(2) the promoter of SEQ ID NO: 4;
(3) a sequence encoding the scaffold sequence of SEQ ID NO: 5;
(4) a sequence encoding the guide sequence of SEQ ID NO: 1;
(5) a sequence encoding the scaffold sequence of SEQ ID NO: 5;
(6) a sequence encoding the guide sequence of SEQ ID NO: 2;
(7) a sequence encoding the scaffold sequence of SEQ ID NO: 5;
(8) the promoter of SEQ ID NO: 6;
(9) Kozak sequence of gccacc;
(10) in-frame start codon ATG;
(11) the first sequence of SEQ ID NO: 8 encoding the first NLS of SEQ ID NO: 15;
(12) the sequence of SEQ ID NO: 33 encoding the Cas13 endonuclease of SEQ ID NO: 32;
(13) the second sequence SEQ ID NO: 10 encoding the second NLS of SEQ ID NO: 15;
(14) an in-frame stop codon;
(15) the WPRE sequence of SEQ ID NO: 11;
(16) the sequence encoding a polyA signal of SEQ ID NO: 12; and
(17) the 3′ ITR of SEQ ID NO: 13;
   wherein the rAAV vector genome comprises an optional linker between any two adjacent components of the components (1) to (17).

In some embodiments, the rAAV vector genome comprises, consists essentially of, or consists of a polynucleotide having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 14. In some embodiments, the polynucleotide encodes an napRNAn of SEQ ID NO: 32 and a guide RNA targeting VEGFA mRNA.

In some embodiments, the rAAV vector genome comprises, from the 5′ to 3′,
(1) the 5′ITR of SEQ ID NO: 3;
(2) the promoter of SEQ ID NO: 4;
(3) a sequence encoding the scaffold sequence of SEQ ID NO: 5;
(4) a sequence encoding the guide sequence of SEQ ID NO: 1;
(5) a sequence encoding the scaffold sequence of SEQ ID NO: 5;
(6) a sequence encoding the guide sequence of SEQ ID NO: 2;
(7) a sequence encoding the scaffold sequence of SEQ ID NO: 5;
(8) the promoter of SEQ ID NO: 6;
(9) Kozak sequence of gccacc;
(10) in-frame start codon ATG;
(11) the first sequence of SEQ ID NO: 8 encoding the first NLS of SEQ ID NO: 15;
(12) the sequence of SEQ ID NO: 33 encoding the napRNAn of SEQ ID NO: 32;
(13) the second sequence SEQ ID NO: 10 encoding the second NLS of SEQ ID NO: 15;
(14) an in-frame stop codon;
(15) the WPRE sequence of SEQ ID NO: 11;
(16) the sequence encoding a polyA signal of SEQ ID NO: 12; and
(17) the 3′ ITR of SEQ ID NO: 13;
   wherein the rAAV vector genome comprises a sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 14;
   wherein the rAAV vector genome does not contain any nucleotide difference from the sequence of SEQ ID NO: 14 in any one of the components (1) to (17);
   wherein the rAAV vector genome contains one or more nucleotide difference or does not contain any nucleotide difference from the sequence of SEQ ID NO: 14 between any two adjacent components of the components (1) to (17).

In some embodiments, the rAAV vector genome comprises the sequence of SEQ ID NO: 14.

In some embodiments, the rAAV vector genome of the disclosure is a DNA rAAV vector genome or an RNA rAAV vector genome. By “DNA rAAV vector genome” it means that the rAAV vector genome is a DNA that can be encapsulated into a rAAV particle. By “RNA rAAV vector genome” it means that the rAAV vector genome is an RNA that can be encapsulated into a rAAV particle.

rAAV Particle

In yet another aspect, the disclosure provides a recombinant AAV (rAAV) particle comprising the rAAV vector genome of the disclosure. A simple introduction of AAV for delivery may refer to “Adeno-associated Virus (AAV) Guide” (addgene.org/guides/aav/).

Adeno-associated virus (AAV), when engineered to delivery, e.g., a protein-encoding sequence of interest, may be termed as a (r)AAV vector, a (r)AAV vector particle, or a (r)AAV particle, where “r” stands for “recombinant”. And the genome packaged in AAV vectors for delivery may be termed as a (r)AAV vector genome, vector genome, or vg for short, while viral genome may refer to the original viral genome of natural AAVs.

The serotypes of the capsids of rAAV particles can be matched to the types of target cells. For example, Table 2 of WO2018002719A1 lists exemplary cell types that can be transduced by the indicated AAV serotypes (incorporated herein by reference).

In some embodiments, the rAAV particle comprising a capsid with a serotype suitable for delivery into retinal cells. In some embodiments, the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV.PHP.eB, a member of the Clade to which any of the AAV1-AAV13 belong, or a functional variant (e.g., a functional truncation) thereof, encapsidating the rAAV vector genome. In some embodiments, the serotype of the capsid is wild type serotype or a functional variant thereof. In some embodiments, the serotype of the capsid is wild type AAV8 or a functional variant thereof.

General principles of rAAV particle production are known in the art. In some embodiments, rAAV particles may be produced using the triple transfection method (described in detail in U.S. Pat. No. 6,001,650).

The vector titers are usually expressed as vector genomes per ml (vg/ml). In some embodiments, the vector titer is above 1×10⁹, above 5×10¹⁰, above 1×10¹¹, above 5×10¹¹, above 1×10¹², above 5×10¹², or above 1×10¹³ vg/ml.

Instead of packaging a single strand (ss) DNA sequence as a vector genome of a rAAV particle, systems and methods of packaging an RNA sequence as a vector genome into a rAAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of rAAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be a functional RNA sequence for use, or an RNA sequence can be produced from the RNA coding sequence, e.g., by RNA processing, or a protein can be translated from the RNA coding sequence.

For example, a Cas13 coding sequence encoding a Cas13 polypeptide covers either a Cas13 DNA coding sequence from which a Cas13 polypeptide is expressed (indirectly via transcription and translation) or a Cas13 RNA coding sequence from which a Cas13 polypeptide is translated (directly).

For example, a gRNA coding sequence encoding a gRNA covers either a gRNA DNA coding sequence from which a gRNA is transcribed or a gRNA RNA coding sequence (1) which per se is the functional gRNA for use, or (2) from which a gRNA is produced, e.g., by RNA processing.

In some embodiments for rAAV RNA vector genomes, 5′-ITR and/or 3′-ITR as DNA packaging signals may be unnecessary and can be omitted at least partly, while RNA packaging signals can be introduced.

In some embodiments for rAAV RNA vector genomes, a promoter to drive transcription of DNA sequences may be unnecessary and can be omitted at least partly.

In some embodiments for rAAV RNA vector genomes, a sequence encoding a polyA signal may be unnecessary and can be omitted at least partly, while a polyA tail can be introduced.

Similarly, other DNA elements of rAAV DNA vector genomes can be either omitted or replaced with corresponding RNA elements and/or additional RNA elements can be introduced, in order to adapt to the strategy of delivering an RNA vector genome by rAAV particles.

In yet another aspect, the disclosure provides a method for production of a rAAV particle, comprising culturing in a host cell a transgene plasmid comprising the rAAV vector genome of the disclosure, thereby encapsulating the rAAV vector genome into a capsid with a serotype of AAV8. In yet another aspect, the disclosure provides a cell comprising a transgene plasmid comprising the rAAV vector genome of the disclosure. In yet another aspect, the disclosure provides use of the cell comprising a transgene plasmid comprising the rAAV vector genome of the disclosure for the production of rAAV particles.

### Pharmaceutical Composition

In yet another aspect, the disclosure provides a pharmaceutical composition comprising (1) the system of the disclosure or the rAAV particle of the disclosure and (2) a pharmaceutically acceptable excipient.

In some embodiments, the pharmaceutical composition comprises the rAAV particle in a concentration selected from the group consisting of about 1×10¹⁰ vg/mL, 2×10¹⁰ vg/mL, 3×10¹⁰ vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical range between any of two preceding values, e.g., in a concentration of from about 9×10¹⁰ vg/mL to about 8×10¹¹ vg/mL.

In some embodiments, the pharmaceutical composition is an injection formulation, such as subretinal injection formulation.

In some embodiments, the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

### Cells

In yet another aspect, the disclosure provides a cell or a progeny thereof comprising the guide nucleic acid of the disclosure, the system of the disclosure, or the rAAV particle of the disclosure. In some embodiments, the cell is a eukaryote. In some embodiments, the cell is a human cell. In some embodiments, the cell is a human retinal cell.

In yet another aspect, the disclosure provides a cell or a progeny thereof comprising VEGFA gene or transcript thereof modified by the system of the disclosure, the rAAV particle of the disclosure, or the method of the disclosure. In some embodiments, the cell is a eukaryote. In some embodiments, the cell is a human cell. In some embodiments, the cell is a human retinal cell.

In some embodiments, the cell is not within the body of an organism, such as, human or animal. In some embodiments, the cell is not a human embryonic stem cell. In some embodiments, the cell is not a human germ cell.

### Treatment Method

In yet another aspect, the disclosure provides a method for preventing, diagnosing, and/or treating a VEGFA associated disease in a subject in need thereof, comprising administering to the subject the system of the disclosure, the rAAV particle of the disclosure, or the pharmaceutical composition of the disclosure, wherein the napBP modifies VEGFA gene or transcript thereof, and wherein the modification of the VEGFA gene or transcript thereof treats the disease. In some embodiments, the napBP modifies the transcript of VEGFA gene, leading to downregulated expression of VEGFA gene, thereby treating the VEGFA associated disease.

In some embodiments, the VEGFA associated disease is neovascular age-related macular degeneration (nAMD) (also known as wet AMD), Diabetic Macular Edema (DME), or a cancer, such as, non-squamous non-small cell lung cancer (NSCLC), renal cell carcinoma (RCC), glioblastoma multiforme, ovarian cancer, and cervical cancer. In some embodiments, the cancer is carcinomas, sarcomas, myelomas, leukemias, lymphomas and mixed type tumors. Non-limiting examples of cancers that may treated by methods and compositions described herein include, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; plasmacytoma, colorectal cancer, rectal cancer, and hairy cell leukemia.

In some embodiments, the VEGFA gene or transcript thereof is in a eukaryotic cell, for example, a human cell, a non-human primate cell, or a mouse cell, such as, a human retinal cell.

In some embodiments, the administrating comprises local administration or systemic administration.

In some embodiments, the administrating comprises subretinal administration, intrathecal administration, intramuscular administration, intravenous administration, transdermal administration, intranasal administration, oral administration, mucosal administration, intraperitoneal administration, intracranial administration, intracerebroventricular administration, or stereotaxic administration.

In some embodiments, the administration is injection or infusion, e.g., subretinal injection.

In some embodiments, the subject is a human, a non-human primate, or a mouse.

In some embodiments, the subject is not responsive to anti-VEGF therapy, such as, anti-VEGF antibody.

In some embodiments, the level of the transcript (e.g., mRNA) of the VEGFA gene is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the VEGFA gene in the subject prior to the administration.

In some embodiments, the level of the expression product (e.g., vascular endothelial growth factor A) of the VEGFA gene is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., vascular endothelial growth factor A) of the VEGFA gene in the subject prior to the administration.

In some embodiments, the formation of choroidal neovascularization (CNV) is inhibited.

In some embodiments, the median survival of the subject suffering from the disease but receiving the administration is 5 days, 10 days, 20 days, 30 days, 2 months, 3 months, 4 months, 5 months, 6 months, 7 months, 8 months, 9 months, 10 months, 11 months, 12 months, 1.5 year, 2 years, 2.5 years, 3 years, 4 years, 5 years, 6 years, 7 years, 8 years, 9 years, 10 years or more longer than that of a subject or a population of subjects suffering from the disease and not receiving the administration.

The dose of the rAAV particle for treatment of the VEGFA associated diseases may be either via a single dose, or multiple doses. One skilled in the art understands that the actual dose may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In some embodiments, the rAAV particle is administrated in a therapeutically effective dose. For example, the therapeutically effective dose of the rAAV particle may be about 1.0E+7 (1.0×10⁷), 2.0E+7, 3.0E+7, 4.0E+7, 6.0E+7, 8.0E+7, 1.0E+8, 2.0E+8, 3.0E+8, 4.0E+8, 6.0E+8, 8.0E+8, 1.0E+9, 2.0E+9, 3.0E+9, 4.0E+9, 6.0E+9, 8.0E+9, 1.0E+10, 2.0E+10, 3.0E+10, 4.0E+10, 6.0E+10, 8.0E+10, 1.0E+11, 2.0E+11, 3.0E+11, 4.0E+11, 6.0E+11, 8.0E+11, 1.0E+12, 2.0E+12, 3.0E+12, 4.0E+12, 6.0E+12, 8.0E+12, 1.0E+13, 2.0E+13, 3.0E+13, 4.0E+13, 6.0E+13, 8.0E+13, 1.0E+14, 2.0E+14, 3.0E+14, 4.0E+14, 6.0E+14, 8.0E+14, 1.0E+15, 2.0E+15, 3.0E+15, 4.0E+15, 6.0E+15, 8.0E+15, 1.0E+16, 2.0E+16, 3.0E+16, 4.0E+16, 6.0E+16, 8.0E+16, or 1.0E+17 vg, or within a range of any two of the those point values. vg stands for vector genomes of rAAV particles for administration. In some embodiments, the therapeutically effective dose of the rAAV particle is in a unit of vg per eye.

### Kit

In yet another aspect, the disclosure provides a kit comprising the system of the disclosure, the rAAV particle of the disclosure, or the pharmaceutical composition of the disclosure, or any one, two, or all components of the same.

In some embodiments, the kit further comprises an instruction to use the component(s) contained therein, and/or instructions for combining with additional component(s) that may be available or necessary elsewhere.

In some embodiments, the kit further comprises one or more buffers that may be used to dissolve any of the component(s) contained therein, and/or to provide suitable reaction conditions for one or more of the component(s). Such buffers may include one or more of PBS, HEPES, Tris, MOPS, Na₂CO₃, NaHCO₃, NaB, or combinations thereof. In some embodiments, the reaction condition includes a proper pH, such as a basic pH. In some embodiments, the pH is between 7-10.

In some embodiments, any one or more of the kit components may be stored in a suitable container or at a suitable temperature, e.g., 4 degree Celsius.

Further embodiments are illustrated in the following Examples which are given for illustrative purposes only and are not intended to limit the scope of the disclosure.

### EXAMPLES

The following examples are provided to further illustrate some embodiments of the disclosure but are not intended to limit the scope of the invention; it will be understood by their exemplary nature that other procedures, methodologies, or techniques known to those skilled in the art may alternatively be used.

### Example 1. In Vitro Knockdown of VEGFA mRNA by napRNAn System

This Example demonstrates the in vitro knockdown efficiency of a napRNAn system of the disclosure comprising a VEGFA mRNA-targeting guide sequence.

The napRNAn system of the disclosure was composed of a napRNAn and a guide RNA (gRNA) comprising a scaffold sequence capable of forming a complex with the napRNAn (compatible with the napRNAn) and a guide sequence designed to hybridize to human VEGFA mRNA. The napRNAn is hfCas13Y (containing D160A, D642A, Y666A, Y677A four mutations; see WO2023/051734 (PCT/CN2022/122833), which is incorporated herein by reference in its entirety). The amino acid sequence of the napRNAn is set forth in SEQ ID NO: 16, and the polynucleotide encoding the napRNAn is set forth in SEQ ID NO: 9. The guide sequence is set forth in SEQ ID NO: 1 or 2 (g1-g2, respectively), and the corresponding target sequence of VEGFA mRNA is set forth in SEQ ID NO: 18 or 19 (T1-T2, respectively). The scaffold sequence is set forth in SEQ ID NO: 5. The gRNA is set forth in SEQ ID NO: 30 or 31 (corresponding to g1-g2, respectively) in a configuration of scaffold-g1-scaffold or scaffold-g2-scaffold (S-G-S) (DR-spacer-DR).

An expression plasmid encoding the VEGFA-targeting gRNA driven by U6 promoter and the napRNAn driven by CBH promoter was transfected into HEK293T cells. An NLS was inserted between the N-terminal Met and the remaining amino acid sequence of the napRNAn. In other words, an NLS coding sequence was inserted between the 5′ In-frame Start Codon ATG and the remaining polynucleotide sequence of the napRNAn coding sequence. The VEGFA knockdown efficiency of the napRNAn system was measured as the fold change of VEGFA mRNA and protein in the transfected HEK293T cells.

hfCas13Y-Mediated RNA Knockdown of VEGFA mRNA In Vitro

It was observed that the designed gRNA along with hfCas13Y showed high expression and significantly reduced VEGFA expression in vitro.

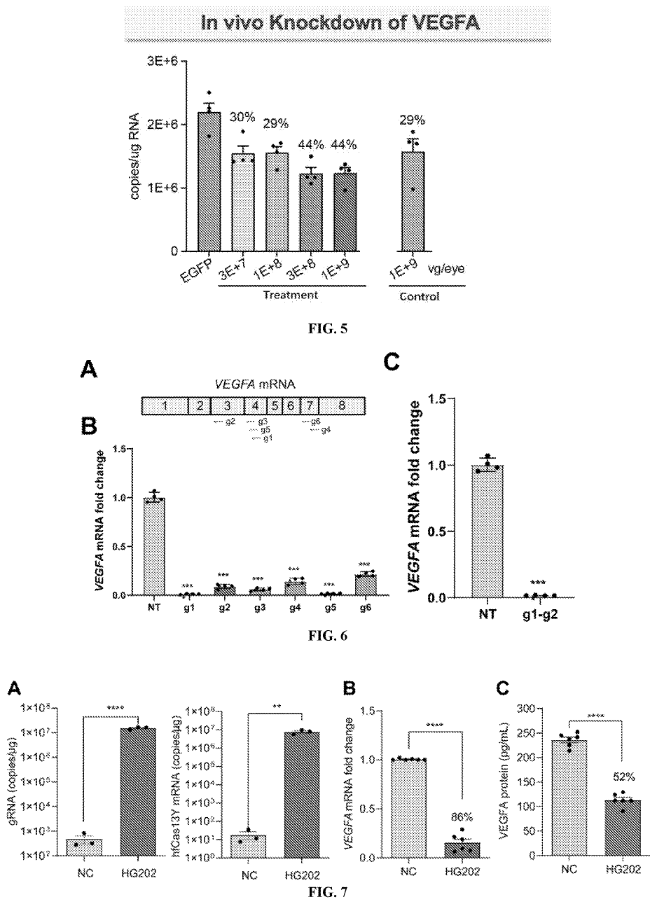

FIG. 6 and FIG. 7 show the results of VEGFA-targeting gRNA screening in vitro. FIG. 6A shows schematic of designed gRNAs (with guide sequences g1-g6) targeting VEGFA mRNA. FIG. 6B shows the knockdown efficiency of hfCas13Y guided by various individual gRNA comprising a VEGFA-targeting guide sequence (one of g1-g6) in HEK293T cells, where gRNA-1 (SEQ ID NO: 30; with guide sequence g1 of SEQ ID NO: 1) and gRNA-2 (SEQ ID NO: 31; with guide sequence g2 of SEQ ID NO: 2) exhibited high VEGFA mRNA knockdown efficiency. NT (nontargeting) means a nontargeting guide sequence incapable of hybridizing to VEGFA mRNA was used in place of the VEGFA targeting guide sequence to serve as a negative control (NC). ***P<0.001.

Then the two guide sequences g1 and g2 were combined, resulting in a gRNA bearing both g1 and g2 as set forth in SEQ ID NO: 17 in a configuration of scaffold-g1-scaffold-g2-scaffold (S-G-S-G-S) (DR-spacer-DR-spacer-DR). It was shown that hfCas13Y along with the gRNA (SEQ ID NO: 17) exhibited ~100% VEGFA mRNA knockdown efficiency in HEK293T cells (FIG. 6C). Both hfCas13Y and the gRNA (SEQ ID NO: 17) were highly expressed in HEK293T cells (FIG. 7A). Both VEGFA mRNA (FIG. 7B) and protein (FIG. 7C) levels were significantly reduced by 86% and 52%, respectively, compared to negative control in HEK293T cells. **P<0.01. ****P<0.0001.

### Off-Target Analysis In Vitro

It was further observed that the combination (denoted as “HG202” in the indicated drawings) of hfCas13Y along with gRNA (SEQ ID NO: 17) showed highly specific targeting and only downregulated VEGFA in vitro, demonstrating its high safety.

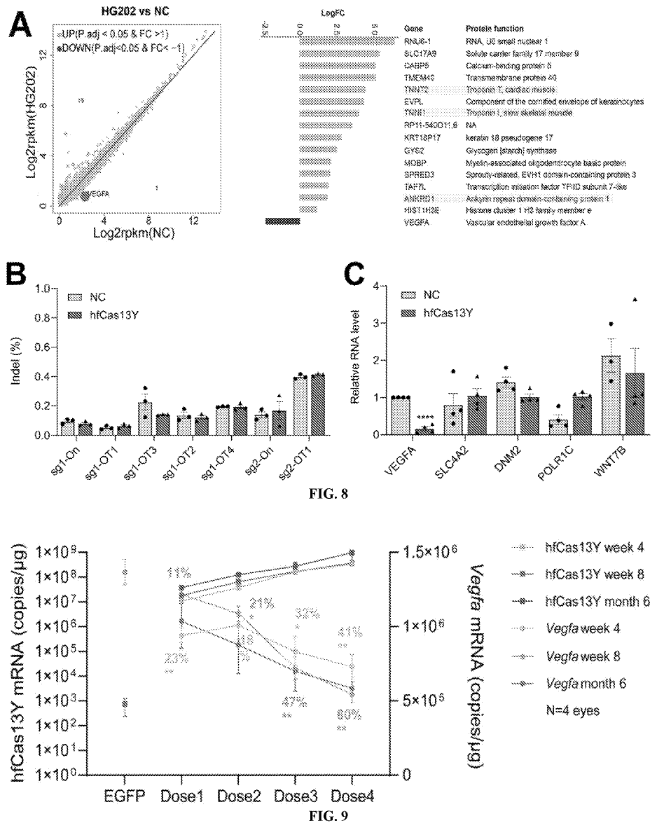

FIG. 8 shows off-target analysis. RNA-seq results exhibited that the HG202 combination specifically targeted and only downregulated VEGFA RNA (FIG. 8A). FIG. 8B shows Indel efficiencies of on-target VEGFA and potential DNA off-target sites. FIG. 8C shows knockdown efficiencies at VEGFA and potential RNA off-target sites. NC=Negative control. ****P<0.0001. Each circle or triangle represents one sample.

### Example 2. In Vivo Knockdown of VEGFA mRNA by AAV-Delivered napRNAn System

This Example demonstrates the in vivo knockdown efficiency of AAV-delivered napRNAn system of the disclosure comprising a VEGFA mRNA-targeting guide sequence.

The napRNAn system of the disclosure was composed of a napRNAn and a gRNA comprising a scaffold sequence capable of forming a complex with the napRNAn (compatible with the napRNAn) and two distinct guide sequences designed to hybridize to human VEGFA mRNA. The napRNAn is hfCas13Y (containing D160A, D642A, Y666A, Y677A four mutations; see WO2023/051734 (PCT/CN2022/122833), which is incorporated herein by reference in its entirety). The amino acid sequence of the napRNAn is set forth in SEQ ID NO: 16, and the polynucleotide encoding the napRNAn is set forth in SEQ ID NO: 9. The two distinct guide sequences are set forth in SEQ ID NO: 1-2 (g1-g2), respectively, and the corresponding target sequences of VEGFA mRNA are set forth in SEQ ID NOs: 18-19 (T1-T2), respectively. The scaffold sequence is set forth in SEQ ID NO: 5. The gRNA is set forth in SEQ ID NO: 17 in a configuration of scaffold-g1-scaffold-g2-scaffold (S-G-S-G-S) (DR-spacer-DR-spacer-DR).

A control napRNAn system according to PCT/CN2022/079890 was used for comparison with the napRNAn system of the disclosure. The control napRNAn system was composed of a napRNAn and a gRNA comprising a scaffold sequence capable of forming a complex with the napRNAn (compatible with the napRNAn) and two distinct guide sequences designed to hybridize to human VEGFA mRNA. The napRNAn is hfCas13X (containing Y672A+Y676A two mutations; see WO2022/068912 (PCT/CN2021/121926), which is incorporated herein by reference in its entirety). The amino acid sequence of the napRNAn is set forth in SEQ ID NO: 27, and the polynucleotide encoding the napRNAn is set forth in SEQ ID NO: 20. The two distinct guide sequences are set forth in SEQ ID NO: 1-2 (g1-g2), respectively, and the corresponding target sequences are set forth in SEQ ID NOs: 18-19 (T1-T2), respectively. The scaffold sequence is set forth in SEQ ID NO: 23. The gRNA is set forth in SEQ ID NO: 25 in a configuration of scaffold-g1-scaffold-g2-scaffold (S-G-S-G-S) (DR-spacer-DR-spacer-DR).

### Transgene Plasmid Construction for AAV Packaging:

For the napRNAn system of the disclosure described above, a treatment rAAV transgene plasmid comprising a treatment rAAV vector genome for packaging into wild type AAV8 capsid to prepare all-in-one treatment rAAV8 particles (named as “HG202”) was designed to comprise, from 5′ to 3′, the elements in Table 1 below. An NLS was inserted between the N-terminal Met and the remaining amino acid sequence of the napRNAn. In other words, an NLS coding sequence was inserted between the 5′ In-frame Start Codon ATG and the remaining polynucleotide sequence of the napRNAn coding sequence.

TABLE 1
Elements of treatment the rAAV vector genome
of the treatment rAAV transgene plasmid

| | | SEQ ID |
|---|---|---|
| Elements | | NO: |
| | AAV2 5′ ITR | 3 |
| | U6 promoter | 4 |
| sequence | sequence encoding hfCas13Y scaffold sequence | 5 |
| encoding | sequence encoding guide sequence g1 | 1 |
| gRNA | sequence encoding hfCas13Y scaffold sequence | 5 |
| (SEQ ID | sequence encoding guide sequence g2 | 2 |
| NO: 17) | sequence encoding hfCas13Y scaffold sequence | 5 |
| | Cbh promoter | 6 |
| | Kozak sequence | 7 |
| | 5′ In-frame start codon ATG | |
| | bpSV40 NLS coding sequence 1 | 8 |
| | napRNAn (hfCas13Y) coding sequence with 5′ start | 33 |
| | codon ATG removed | |
| | bpSV40 NLS coding sequence 2 | 10 |
| | In-frame stop codon | |
| | WPRE3 | 11 |
| | sequence encoding polyA signal | 12 |
| | AAV2 3′ ITR | 13 |

The full length of the 5′ ITR-to-3′ ITR sequence of the treatment rAAV vector genome of the treatment rAAV transgene plasmid is set forth in SEQ ID NO: 14.

For the control napRNAn system described above, a control rAAV transgene plasmid comprising a control rAAV vector genome for packaging into wild type AAV9 capsid to prepare all-in-one control rAAV9 particles (named as “HG203” or “HG-203”) was designed to comprise, from 5′ to 3′, the elements in Table 2 below. An NLS was inserted between the N-terminal Met and the remaining amino acid sequence of the napRNAn. In other words, an NLS coding sequence was inserted between the 5′ In-frame Start Codon ATG and the remaining polynucleotide sequence of the napRNAn coding sequence.

TABLE 2
Elements of the control rAAV vector genome
of the control rAAV transgene plasmid

| | | SEQ ID |
|---|---|---|
| Elements | | NO: |
| | AAV2 5′ ITR | 28 |
| | EFS promoter | 18 |
| | Kozak sequence | 7 |
| | 5′ In-frame start codon ATG | |
| | SV40 NLS coding sequence | 19 |
| | napRNAn (hfCas13X) coding sequence with 5′ start | 35 |
| | codon ATG removed | |
| | SV40 NLS coding sequence | 19 |
| | In-frame stop codon | |
| | sequence encoding polyA signal | 21 |
| | U6 promoter | 22 |
| sequence | sequence encoding hfCas13X scaffold sequence | 23 |
| encoding | sequence encoding guide sequence g1 | 1 |
| gRNA | sequence encoding hfCas13X scaffold sequence | 23 |
| (SEQ ID | sequence encoding guide sequence g2 | 2 |
| NO: 25) | sequence encoding hfCas13X scaffold sequence | 23 |
| | AAV2 3′ ITR | 29 |

The full length of the 5′ ITR-to-3′ ITR sequence of the control rAAV vector genome of the control rAAV transgene plasmid is set forth in SEQ ID NO: 24.

rAAV Particle Preparation:

The treatment rAAV8 particles (HG202) with wild type AAV8 capsid and the control rAAV9 particles (HG203) with wild type AAV9 capsid were produced using conventional triple-plasmid transfection system mutatis mutandis, by co-transfecting the treatment or control rAAV transgene plasmid, packaging plasmid, and helper plasmid in a weight ratio of 1:1:2 into HEK293T cells. The treatment or control rAAV transgene plasmid was packaged by AAV8 or AAV9 capsid to form the genome inside the capsid, and together the genome and the AAV8 or AAV9 capsid constituted rAAV8 or rAAV9 particles.

Specifically, the HEK293T cells were cultured in competent DMEM medium, and the cells were plated 24 hours before transfection of the plasmids. Shortly before transfection, the culture medium was replaced with fresh DMEM containing 2% FBS. PEI-MAX was used as the transfection reagent. The transfected HEK293T cells were harvested from the media at 72 hours post translation. The rAAV particles were purified from the cells by using iodixanol density gradient ultracentrifugation.

### Experimental Procedure:

The laser induced CNV mouse model, rAAV particle administration by subretinal injection (FIG. 13), and knockdown detection (e.g., inhibition of CNV growth) were established and conducted according to the Examples of PCT/CN2022/079890 unless otherwise indicated herein.

### Results:

### Comparison of VEGFA Knockdown Efficiency of HG202 and HG203

The treatment rAAV8 particles HG202 (with wt AAV8 capsid and ITR-to-ITR sequence of SEQ ID NO: 14) differs from the control rAAV9 particles HG203 (with wt AAV9 capsid and ITR-to-ITR sequence of SEQ ID NO: 24) in various aspects, including, among others, the selection of wt AAV8 capsid over wt AAV9 capsid, the selection of hfCas13Y (and compatible scaffold sequence) over hfCas13X (and compatible scaffold sequence), the selection of Cbh promoter (SEQ ID NO: 6) for expressing hfCas13Y over EFS promoter (SEQ ID NO: 18) for expressing hfCas13X, the selection of NLS and polyA signal, and the selection of the positional relationship of the gRNA expression cassette and the Cas13 expression cassette in 5′-3′ or 3′-5′ direction.

Referring to FIG. 5, the in vivo mVEGFA mRNA knockdown results show that:
(1) at the same does of 1E9 vg/eye, the mVEGFA mRNA knockdown efficiency of the treatment rAAV8 particles (44%, 1E9 vg/eye) was significantly higher than that of the control rAAV9 particles (29%, 1E9 vg/eye);
(2) even at about 1/3.3 dose, the mVEGFA mRNA knockdown efficiency of the treatment rAAV8 particles (44%, 3E8 vg/eye) was still significantly higher than that of the control rAAV9 particles (29%, 1E9 vg/eye);
(3) even at about 1/10 dose, the mVEGFA mRNA knockdown efficiency of the treatment rAAV8 particles (29%, 1E8 vg/eye) was still comparable to that of the control rAAV9 particles (29%, 1E9 vg/eye); and
(4) even at about 1/33 dose, the mVEGFA mRNA knockdown efficiency of the treatment rAAV8 particles (30%, 3E7 vg/eye) was still comparable to that of the control rAAV9 particles (29%, 1E9 vg/eye).

Overall, the knockdown results demonstrate that the treatment rAAV8 particles HG202 achieved a significant effect of knocking down VEGFA mRNA in vivo, and in particular, achieved significantly improved knockdown efficiency than the control rAAV9 particles HG203 as reflected by the significantly higher knockdown efficiency at about 1/3.3 dose and comparable knockdown efficiency at about 1/10 dose to about 1/33 dose, which also highlights the higher safety of the treatment rAAV8 particles HG202 compared to the control rAAV9 particles HG203 due to the reduced dose for effectiveness.

HG202-Mediated RNA Knockdown of Vegfa mRNA In Vivo

It was observed that HG202 highly expressed hfCas13Y mRNA and downregulated Vegfa mRNA in the treated mouse eyes.

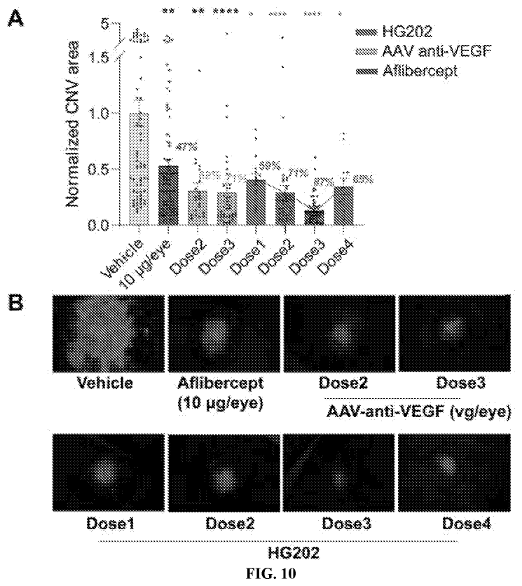

FIG. 9 shows VEGFA knockdown in vivo. Subretinal injection of HG202 at different doses resulted in significant dose-related expression of hfCas13Y mRNA in the treated mouse eyes, while a dose-dependent decrease of Vegfa mRNA expression was observed. Dose 1=3E+7 vg/eye; Dose 2=1E+8 vg/eye; Dose 3=3E+8 vg/eye; Dose 4=1E+9 vg/eye.

### CNV (Choroidal Neovascularization) Inhibition Efficiency of HG202 in Mice

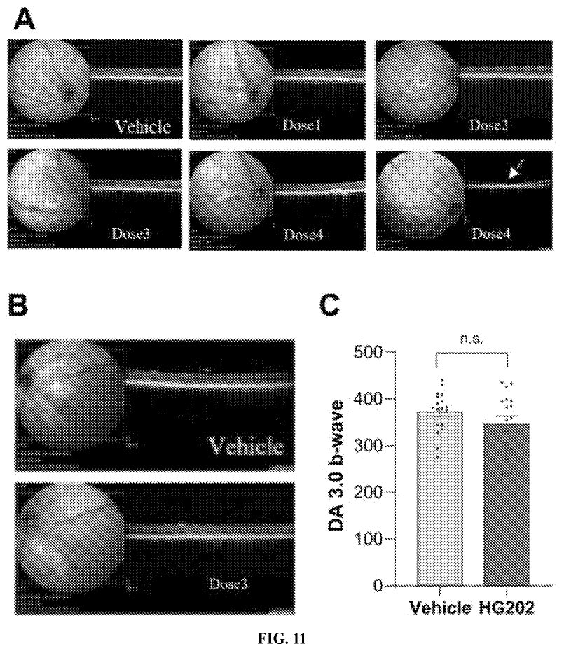

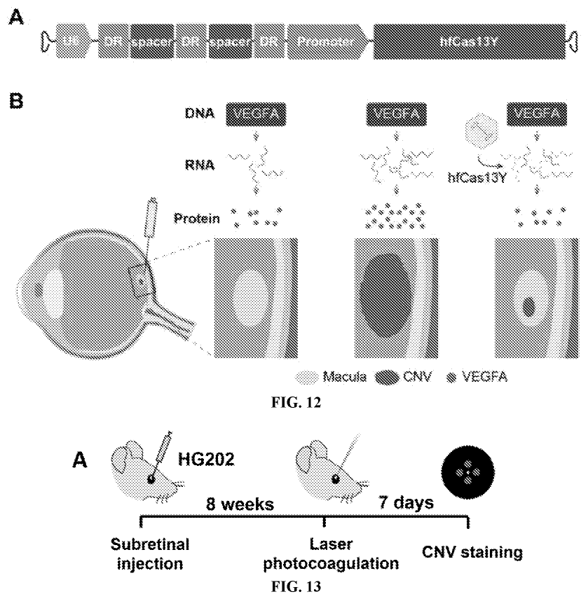

FIG. 10 shows reduction of CNV in vivo by HG202. The area of laser-induced CNV was markedly reduced in the mouse eyes treated with HG202 at Week 8 (FIG. 10A). Moreover, the reduction of CNV by HG202 at all the doses (59% to 87%) was superior to that by Aflibercept (47%), an anti-VEGF agent. FIG. 10B are representative images of CNV staining at different doses of HG202. *P<0.05; **P<0.01; ****P<0.0001.

### HG202 was Well Tolerated in Mice

It was observed that HG202 showed no significant impact on retinal structure and function at effective doses.

FIG. 11 shows safety of HG202. OCT results show that there was no significant impact on retinal structure 8 weeks after subretinal injection of HG202 at Dose 1-3 (FIG. 11A). OCT (FIG. 11B) and ERG (FIG. 11C) results show that there was no significant change of retinal structure and function 6 months after subretinal injection of HG202 at Dose 3. NS=No Significance; DA=Dark Adapted.

It is indicated that the delivery of the hfCas13Y system targeting VEGFA mRNA with rAAV8 particles may be a promising therapy for treating VEGFA associated diseases, such as, wet AMD, Diabetic Macular Edema (DME). The single subretinal injection of HG202 enables long-term transgene expression and RNA editing, resulting in the elimination of 1- or 2-month repeated injection and providing long-lasting therapeutic benefits for patients (with or without anti-VEGF antibody) to treat nAMD and DME.

Various modifications and variations of the described products, methods, and uses of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### Exemplary Sequences

### Guide Sequence, SEQ ID NO: 1-2

| | Guide | SEQ | gRNA | SEQ | | Target | SEQ |
|---|---|---|---|---|---|---|---|
| | sequence | ID | sequence | ID | | sequence | ID |
| No. | (5′-3′) | NO: | (5′-3′) | NO: | No. | (5′-3′) | NO: |
| g1 | GTGCTGTA | 1 | GCTGTGAT | 30 | T1 | CACATAG | 18 |
| | GGAAGCTC | | AGACCTCG | | | GAGAGAT | |
| | ATCTCTCC | | ATTTGTGG | | | GAGCTTC | |
| | TATGTG | | GGTAGTAA | | | CTACAGC | |
| | | | CAGCGTGC | | | AC | |
| | | | TGTAGGAA | | | | |
| | | | GCTCATCT | | | | |
| | | | CTCCTATG | | | | |
| | | | TGGCTGTG | | | | |
| | | | ATAGACCT | | | | |
| | | | CGATTTGT | | | | |
| | | | GGGGTAGT | | | | |
| | | | AACAGC | | | | |
| g2 | GGTACTCC | 2 | GCTGTGAT | 31 | T2 | AGACCCT | 19 |
| | TGGAAGAT | | AGACCTCG | | | GGTGGAC | |
| | GTCCACCA | | ATTTGTGG | | | ATCTTCC | |
| | GGGTCT | | GGTAGTAA | | | AGGAGTA | |
| | | | CAGCGGTA | | | CC | |
| | | | CTCCTGGA | | | | |
| | | | AGATGTCC | | | | |
| | | | ACCAGGGT | | | | |
| | | | CTGCTGTG | | | | |
| | | | ATAGACCT | | | | |
| | | | CGATTTGT | | | | |
| | | | GGGGTAGT | | | | |
| | | | AACAGC | | | | |

AAV2 5′ ITR sequence,
SEQ ID NO: 3
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCT
U6 promoter sequence,
SEQ ID NO: 4
GAGGGCCTATTTCCCATGATTCCTTCATATTTGCATATACGATACAAGGCTGTTAGAGAGATAATTAGAA
TTAATTTGACTGTAAACACAAAGATATTAGTACAAAATACGTGACGTAGAAAGTAATAATTTCTTGGGT
AGTTTGCAGTTTTAAAATTATGTTTTAAAATGGACTATCATATGCTTACCGTAACTTGAAAGTATTTCGA
TTTCTTGGCTTTATATATCTTGTGGAAAGGACGAAACACCG
hfCas13Y scaffold sequence,
SEQ ID NO: 5
GCTGTGATAGACCTCGATTTGTGGGGTAGTAACAGC
Cbh promoter,
SEQ ID NO: 6
CGTTACATAACTTACGGTAAATGGCCCGCCTGGCTGACCGCCCAACGACCCCCGCCCATTGACGTCAA
TAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGCC
TGGCATTGTGCCCAGTACATGACCTTATGGGACTTTCCTACTTGGCAGTACATCTACGTATTAGTCATCG
CTATTACCATGGTCGAGGTGAGCCCCACGTTCTGCTTCACTCTCCCCATCTCCCCCCCCTCCCCACCCC
CAATTTTGTATTTATTTATTTTTTAATTATTTTGTGCAGCGATGGGGGCGGGGGGGGGGGGGGGGCGCG
CGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTATAAA
AAGCGAAGCGCGCGGCGGGCGGGAGTCGCTGCGCGCTGCCTTCGCCCCGTGCCCCGCTCCGCCGCC
GCCTCGCGCCGCCCGCCCCGGCTCTGACTGACCGCGTTACTCCCACAGGTGAGCGGGCGGGACGGCC
CTTCTCCTCCGGGCTGTAATTAGCTGAGCAAGAGGTAAGGGTTTAAGGGATGGTTGGTTGGTGGGGTA
TTAATGTTTAATTACCTGGAGCACCTGCCTGAAATCACTTTTTTTCAGGTTGG
Kozak sequence,
SEQ ID NO: 7
GCCACC
bpSV40 NLS coding sequence 1,
SEQ ID NO: 8
AAGCGCACAGCCGACGGCAGCGAGTTTGAAAGCCCTAAGAAGAAAAGAAAGGTG
napRNAn (hfCas13Y) coding sequence,
SEQ ID NO: 9
ATGAACGGAATCGAGCTGAAGAAGGAGGAAGCCGCCTTTTACTTCAATCAGGCCGAGCTGAACTTAA
AGGCCATCGAGGACAACATCTTTGACAAGGAGCGGAGAAAGACCCTGCTGAATAACCCTCAGATCCT
GGCCAAGATGGAGAACTTCATATTCAACTTCAGAGATGTGACAAAGAACGCCAAGGGCGAGATTGAC
TGCCTGCTGCTGAAACTGAGAGAACTGAGAAACTTCTACAGCCACTACGTGCACAAGAGAGACGTG
CGGGAACTATCTAAAGGCGAGAAGCCTATCCTGGAGAAGTATTACCAGTTTGCCATCGAAAGCACCG
GCAGCGAAAACGTCAAGCTGGAAATCATCGAGAATGACGCTTGGCTGGCTGATGCCGGGGTGCTGTT
TTTCCTGTGCATCTTCCTGAAGAAGTCCCAAGCCAACAAGCTCATTAGCGGCATCAGCGGCTTCAAGA
GAAACGACGCTACCGGCCAGCCAAGAAGAAATCTGTTCACCTACTTCAGCATCAGAGAAGGCTACAA
GGTGGTGCCCGAGATGCAGAAGCACTTCCTGCTGTTCTCTCTGGTGAACCACCTGAGCAATCAGGAC
GACTATATCGAGAAGGCCCACCAACCCTACGACATCGGAGAGGGCCTGTTCTTCCACAGAATCGCCA
GCACCTTCCTGAACATCAGCGGCATCCTGAGAAACATGAAATTCTACACATACCAAAGCAAAAGACT
GGTCGAGCAGAGAGGCGAGCTGAAAAGGGAGAAGGACATTTTCGCCTGGGAAGAGCCTTTCCAGGG
CAACAGCTACTTCGAGATCAATGGCCACAAAGGAGTTATCGGCGAAGATGAACTGAAGGAACTGTGT
TACGCCTTCCTGATCGGCAATCAAGACGCCAACAAGGTAGAAGGCAGAATCACACAGTTCCTGGAAA
AGTTCAGAAACGCCAACAGCGTGCAGCAGGTGAAGGACGATGAAATGCTGAAGCCTGAATACTTTCC
TGCAAACTACTTTGCCGAGAGCGGTGTGGGCAGAATCAAGGACCGGGTGCTGAACCGGCTGAACAA
GGCCATAAAGAGCAATAAAGCTAAGAAGGGCGAAATCATCGCCTACGATAAGATGCGGGAAGTGATG
GCCTTCATCAACAACAGCCTACCAGTGGATGAGAAGCTGAAGCCCAAGGATTACAAGCGGTACCTGG
GAATGGTCCGGTTCTGGGATAGAGAGAAGGATAACATCAAGCGCGAATTCGAGACAAAAGAGTGGTC
CAAATACCTGCCTAGCAATTTCTGGACCGCCAAGAACCTGGAGCGGGTGTACGGACTGGCCCGTGAG
AAAAACGCAGAGCTGTTCAACAAGCTGAAAGCTGACGTGGAAAAAATGGATGAGAGAGAACTGGA
GAAATACCAGAAGATCAACGACGCCAAAGACCTGGCTAACCTGCGGCGGCTGGCCTCTGACTTCGGC
GTGAAGTGGGAAGAGAAGGACTGGGACGAGTACTCTGGCCAGATCAAGAAGCAGATCACCGACAGC
CAGAAGCTGACCATCATGAAGCAGAGAATTACCGCAGGCCTGAAAAAAAAGCACGGCATCGAAAAC
CTGAACCTGAGAATCACCATCGACATCAATAAGAGCAGAAAAGCCGTGCTGAATAGAATTGCCATCCC
TAGAGGCTTTGTGAAGAGACATATCCTCGGATGGCAGGAGTCTGAGAAGGTGTCCAAAAAAATCAGA
GAAGCCGAGTGCGAGATCCTGCTCTCCAAGGAGTATGAAGAGCTGAGCAAACAGTTCTTCCAGAGCA
AGGACTACGACAAAATGACCAGAATCAACGGCCTGTACGAGAAAAACAAGCTTATCGCCCTGATGGC
CGTGTACCTGATGGGCCAGCTGCGGATCCTGTTTAAGGAACACACAAAGCTGGCTGATATCACAAAA
ACCACAGTGGATTTCAAGATCAGCGACAAGGTGACAGTGAAGATTCCCTTCAGCAACGCTCCTAGCC
TGGTGTATACCATGAGCAGCAAGGCCGTGGACAACATCGGAAATTACGGCTTCAGCAACAAAGACAA
GGATAAGCCCATCCTGGGCAAGATCGATGTGATCGAGAAGCAGCGGATGGAGTTTATCAAGGAAGTC
CTGGGATTCGAGAAGTACCTGTTCGATGATAAGATCATCGACAAGAGCAAGTTCGCCGACACCGCCA
CCCACATCTCTTTCGCCGAGATCGTTGAAGAGCTGGTGGAAAAGGGATGGGACAAAGATCGGCTGAC
CAAGCTGAAGGACGCTAGAAACAAGGCTCTGCATGGCGAAATCCTGACCGGCACCAGCTTCGACGA
GACAAAGTCCCTGATCAACGAGCTGAAGAAG
bpSV40 NLS coding sequence 2,
SEQ ID NO: 10
AAGAGAACCGCCGACGGCTCTGAGTTCGAAAGTCCTAAGAAGAAGCGGAAGGTG
WPRE3 sequence,
SEQ ID NO: 11
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTCC
TTGTATAAATCCTGGTTAGTTCTTGCCACGGCGGAACTCATCGCCGCCTGCCTTGCCCGCTGCTGGAC
AGGGGCTCGGCTGTTGGGCACTGACAATTCCGTGGTGTT
Sequence encoding bGH polyA signal,
SEQ ID NO: 12
CTGTGCCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCATTCTA
TTCTGGGGGGTGGGGTGGGGCAGGACAGCAAGGGGGAGGATTGGGAAGACAATAGCAGGCATGCTG
GGGATGCGGTGGGCTCTATGG
AAV2 3′ ITR sequence,
SEQ ID NO: 13
AGGAACCCCTAGTGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCGAGCGCGCAG
ITR-to-ITR sequence,
SEQ ID NO: 14
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCG
GCCATCGATACTAGTAAGGTCGGGCAGGAAGAGGGCCTATTTCCCATGATTCCTTCATATTTGCATATAC
GATACAAGGCTGTTAGAGAGATAATTAGAATTAATTTGACTGTAAACACAAAGATATTAGTACAAAATA
CGTGACGTAGAAAGTAATAATTTCTTGGGTAGTTTGCAGTTTTAAAATTATGTTTTAAAATGGACTATC
ATATGCTTACCGTAACTTGAAAGTATTTCGATTTCTTGGCTTTATATATCTTGTGGAAAGGACGAAACAC
CGGCTGTGATAGACCTCGATTTGTGGGGTAGTAACAGCGTGCTGTAGGAAGCTCATCTCTCCTATGTG
GCTGTGATAGACCTCGATTTGTGGGGTAGTAACAGCGGTACTCCTGGAAGATGTCCACCAGGGTCTGC
TGTGATAGACCTCGATTTGTGGGGTAGTAACAGCTTTTTTTTACGCGCCCGTTACATAACTTACGGTAA
ATGGCCCGCCTGGCTGACCGCCCAACGACCCCCGCCCATTGACGTCAATAGTAACGCCAATAGGGACT
TTCCATTGACGTCAATGGGTGGAGTATTTACGGTAAACTGCCCACTTGGCAGTACATCAAGTGTATCAT
ATGCCAAGTACGCCCCCTATTGACGTCAATGACGGTAAATGGCCCGCCTGGCATTGTGCCCAGTACAT
GACCTTATGGGACTTTCCTACTTGGCAGTACATCTACGTATTAGTCATCGCTATTACCATGGTCGAGGTG
AGCCCCACGTTCTGCTTCACTCTCCCCATCTCCCCCCCCTCCCCACCCCCAATTTTGTATTTATTTATTT
TTTAATTATTTTGTGCAGCGATGGGGGCGGGGGGGGGGGGGGGGCGCGCGCCAGGCGGGGCGGGGC
GGGGCGAGGGGCGGGGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGCGAAGCGCGCGGCGGG
CGGGAGTCGCTGCGCGCTGCCTTCGCCCCGTGCCCCGCTCCGCCGCCGCCTCGCGCCGCCCGCCCCG
GCTCTGACTGACCGCGTTACTCCCACAGGTGAGCGGGCGGGACGGCCCTTCTCCTCCGGGCTGTAAT
TAGCTGAGCAAGAGGTAAGGGTTTAAGGGATGGTTGGTTGGTGGGGTATTAATGTTTAATTACCTGGA
GCACCTGCCTGAAATCACTTTTTTTCAGGTTGGACCGGTGCCACCATGAAGCGCACAGCCGACGGCA
GCGAGTTTGAAAGCCCTAAGAAGAAAAGAAAGGTGAACGGAATCGAGCTGAAGAAGGAGGAAGCC
GCCTTTTACTTCAATCAGGCCGAGCTGAACTTAAAGGCCATCGAGGACAACATCTTTGACAAGGAGC
GGAGAAAGACCCTGCTGAATAACCCTCAGATCCTGGCCAAGATGGAGAACTTCATATTCAACTTCAG
AGATGTGACAAAGAACGCCAAGGGCGAGATTGACTGCCTGCTGCTGAAACTGAGAGAACTGAGAAA
CTTCTACAGCCACTACGTGCACAAGAGAGACGTGCGGGAACTATCTAAAGGCGAGAAGCCTATCCTG
GAGAAGTATTACCAGTTTGCCATCGAAAGCACCGGCAGCGAAAACGTCAAGCTGGAAATCATCGAGA
ATGACGCTTGGCTGGCTGATGCCGGGGTGCTGTTTTTCCTGTGCATCTTCCTGAAGAAGTCCCAAGCC
AACAAGCTCATTAGCGGCATCAGCGGCTTCAAGAGAAACGACGCTACCGGCCAGCCAAGAAGAAAT
CTGTTCACCTACTTCAGCATCAGAGAAGGCTACAAGGTGGTGCCCGAGATGCAGAAGCACTTCCTGC
TGTTCTCTCTGGTGAACCACCTGAGCAATCAGGACGACTATATCGAGAAGGCCCACCAACCCTACGAC
ATCGGAGAGGGCCTGTTCTTCCACAGAATCGCCAGCACCTTCCTGAACATCAGCGGCATCCTGAGAA
ACATGAAATTCTACACATACCAAAGCAAAAGACTGGTCGAGCAGAGAGGCGAGCTGAAAAGGGAGA
AGGACATTTTCGCCTGGGAAGAGCCTTTCCAGGGCAACAGCTACTTCGAGATCAATGGCCACAAAGG
AGTTATCGGCGAAGATGAACTGAAGGAACTGTGTTACGCCTTCCTGATCGGCAATCAAGACGCCAAC
AAGGTAGAAGGCAGAATCACACAGTTCCTGGAAAAGTTCAGAAACGCCAACAGCGTGCAGCAGGTG
AAGGACGATGAAATGCTGAAGCCTGAATACTTTCCTGCAAACTACTTTGCCGAGAGCGGTGTGGGCA
GAATCAAGGACCGGGTGCTGAACCGGCTGAACAAGGCCATAAAGAGCAATAAAGCTAAGAAGGGCG
AAATCATCGCCTACGATAAGATGCGGGAAGTGATGGCCTTCATCAACAACAGCCTACCAGTGGATGAG
AAGCTGAAGCCCAAGGATTACAAGCGGTACCTGGGAATGGTCCGGTTCTGGGATAGAGAGAAGGATA
ACATCAAGCGCGAATTCGAGACAAAAGAGTGGTCCAAATACCTGCCTAGCAATTTCTGGACCGCCAA
GAACCTGGAGCGGGTGTACGGACTGGCCCGTGAGAAAAACGCAGAGCTGTTCAACAAGCTGAAAGC
TGACGTGGAAAAAATGGATGAGAGAGAACTGGAGAAATACCAGAAGATCAACGACGCCAAAGACCT
GGCTAACCTGCGGCGGCTGGCCTCTGACTTCGGCGTGAAGTGGGAAGAGAAGGACTGGGACGAGTA
CTCTGGCCAGATCAAGAAGCAGATCACCGACAGCCAGAAGCTGACCATCATGAAGCAGAGAATTACC
GCAGGCCTGAAAAAAAAGCACGGCATCGAAAACCTGAACCTGAGAATCACCATCGACATCAATAAG
AGCAGAAAAGCCGTGCTGAATAGAATTGCCATCCCTAGAGGCTTTGTGAAGAGACATATCCTCGGATG
GCAGGAGTCTGAGAAGGTGTCCAAAAAAATCAGAGAAGCCGAGTGCGAGATCCTGCTCTCCAAGGA
GTATGAAGAGCTGAGCAAACAGTTCTTCCAGAGCAAGGACTACGACAAAATGACCAGAATCAACGG
CCTGTACGAGAAAAACAAGCTTATCGCCCTGATGGCCGTGTACCTGATGGGCCAGCTGCGGATCCTGT
TTAAGGAACACACAAAGCTGGCTGATATCACAAAAACCACAGTGGATTTCAAGATCAGCGACAAGGT
GACAGTGAAGATTCCCTTCAGCAACGCTCCTAGCCTGGTGTATACCATGAGCAGCAAGGCCGTGGAC
AACATCGGAAATTACGGCTTCAGCAACAAAGACAAGGATAAGCCCATCCTGGGCAAGATCGATGTGA
TCGAGAAGCAGCGGATGGAGTTTATCAAGGAAGTCCTGGGATTCGAGAAGTACCTGTTCGATGATAA
GATCATCGACAAGAGCAAGTTCGCCGACACCGCCACCCACATCTCTTTCGCCGAGATCGTTGAAGAG
CTGGTGGAAAAGGGATGGGACAAAGATCGGCTGACCAAGCTGAAGGACGCTAGAAACAAGGCTCTG
CATGGCGAAATCCTGACCGGCACCAGCTTCGACGAGACAAAGTCCCTGATCAACGAGCTGAAGAAG
TCCGGCGGCTCTAAGAGAACCGCCGACGGCTCTGAGTTCGAAAGTCCTAAGAAGAAGCGGAAGGTG
TGAGCTAGCAATCAACCTCTGGATTACAAAATTTGTGAAAGATTGACTGGTATTCTTAACTATGTTGCT
CCTTTTACGCTATGTGGATACGCTGCTTTAATGCCTTTGTATCATGCTATTGCTTCCCGTATGGCTTTCAT
TTTCTCCTCCTTGTATAAATCCTGGTTAGTTCTTGCCACGGCGGAACTCATCGCCGCCTGCCTTGCCCG
CTGCTGGACAGGGGCTCGGCTGTTGGGCACTGACAATTCCGTGGTGTTCTGTGCCTTCTAGTTGCCAG
CCATCTGTTGTTTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGGGGTGGGGTGGG
GCAGGACAGCAAGGGGGAGGATTGGGAAGACAATAGCAGGCATGCTGGGGATGCGGTGGGCTCTAT
GGGCGGCCGCAGGAACCCCTAGTGATGGAGTTGGCCACTCCCTCTCTGCGCGCTCGCTCGCTCACTG
AGGCCGGGCGACCAAAGGTCGCCCGACGCCCGGGCTTTGCCCGGGCGGCCTCAGTGAGCGAGCGAG
CGCGCAG
bpSV40 NLS amino acid sequence,
SEQ ID NO: 15
KRTADGSEFESPKKKRKV
napRNAn (hfCas13Y) amino acid sequence,
SEQ ID NO: 16
MNGIELKKEEAAFYFNQAELNLKAIEDNIFDKERRKTLLNNPQILAKMENFIFNFRDVTKNAKGEIDCLLL
KLRELRNFYSHYVHKRDVRELSKGEKPILEKYYQFAIESTGSENVKLEIIENDAWLADAGVLFFLCIFLKK
SQANKLISGISGFKRNDATGQPRRNLFTYFSIREGYKVVPEMQKHFLLFSLVNHLSNQDDYIEKAHQPYDI
GEGLFFHRIASTFLNISGILRNMKFYTYQSKRIVEQRGELKREKDIFAWEEPFQGNSYFEINGHKGVIGEDE
LKELCYAFLIGNQDANKVEGRITQFLEKFRNANSVQQVKDDEMLKPEYFPANYFAESGVGRIKDRVLNRL
NKAIKSNKAKKGEIIAYDKMREVMAFINNSLPVDEKLKPKDYKRYLGMVRFWDREKDNIKREFETKEWS
KYLPSNFWTAKNLERVYGLAREKNAELFNKLKADVEKMDERELEKYQKINDAKDLANLRRLASDFGVK
WEEKDWDEYSGQIKKQITDSQKLTIMKQRITAGLKKKHGIENLNLRITIDINKSRKAVLNRIAIPRGFVKRH
ILGWQESEKVSKKIREAECEILLSKEYEELSKQFFQSKDYDKMTRINGLYEKNKLIALMAVYLMGQLRILF
KEHTKLADITKTTVDFKISDKVTVKIPFSNAPSLVYTMSSKAVDNIGNYGFSNKDKDKPILGKIDVIEKQR
MEFIKEVLGFEKYLFDDKIIDKSKFADTATHISFAEIVEELVEKGWDKDRLTKLKDARNKALHGEILTGTSF
DETKSLINELKK
gRNA (S-g1-S-g2-S) for hfCas13Y,
SEQ ID NO: 17
GCTGTGATAGACCTCGATTTGTGGGGTAGTAACAGCGTGCTGTAGGAAGCTCATCTCTCCTATGTGGC
TGTGATAGACCTCGATTTGTGGGGTAGTAACAGCGGTACTCCTGGAAGATGTCCACCAGGGTCTGCTG
TGATAGACCTCGATTTGTGGGGTAGTAACAGC
EFS promoter,
SEQ ID NO: 18
TAGGTCTTGAAAGGAGTGGGAATTGGCTCCGGTGCCCGTCAGTGGGCAGAGCGCACATCGCCCACAG
TCCCCGAGAAGTTGGGGGGAGGGGTCGGCAATTGATCCGGTGCCTAGAGAAGGTGGCGCGGGGTAA
ACTGGGAAAGTGATGTCGTGTACTGGCTCCGCCTTTTTCCCGAGGGTGGGGGAGAACCGTATATAAGT
GCAGTAGTCGCCGTGAACGTTCTTTTTCGCAACGGGTTTGCCGCCAGAACACAGGGTTGG
SV40 coding sequence,
SEQ ID NO: 19
CCCAAGAAGAAGCGGAAGGTG
napRNAn (hfCas13X) coding sequence,
SEQ ID NO: 20
ATGGCCCAGGTGAGCAAGCAGACCTCCAAGAAGAGGGAGCTGAGCATCGACGAGTACCAGGGCGCC
CGGAAGTGGTGCTTCACCATTGCCTTCAACAAGGCCCTGGTGAACCGGGACAAGAACGACGGCCTGT
TCGTGGAAAGCCTGCTGAGACACGAGAAGTACAGCAAGCACGACTGGTACGACGAAGATACCCGGG
CCCTGATCAAGTGCAGCACCCAGGCCGCCAACGCCAAGGCTGAAGCCCTGCGGAACTACTTCAGTCA
CTACCGGCATAGCCCTGGCTGCCTGACCTTCACCGCCGAGGACGAACTGCGGACCATCATGGAGAGA
GCCTATGAGCGGGCCATCTTCGAGTGCAGAAGAAGAGAGACAGAGGTGATCATCGAGTTTCCCAGCC
TGTTCGAGGGCGACCGGATCACCACCGCCGGCGTGGTGTTTTTCGTGAGCTTTTTCGTGGAAAGAAG
AGTGCTGGATCGGCTGTATGGAGCCGTGTCCGGCCTGAAGAAGAATGAGGGACAGTACAAGCTGACC
CGGAAGGCCCTGAGCATGTACTGCCTGAAGGACAGCAGATTCACCAAGGCCTGGGATAAGCGGGTGC
TGCTGTTCAGAGACATCCTGGCCCAGCTGGGAAGAATCCCCGCCGAGGCCTACGAGTACTACCACGG
CGAGCAGGGTGATAAGAAGAGAGCTAACGACAATGAGGGCACAAATCCCAAGCGGCACAAGGACAA
GTTCATCGAATTTGCACTGCACTACCTGGAAGCCCAGCACAGCGAGATCTGCTTCGGCAGACGCCAC
ATCGTGCGGGAAGAGGCCGGCGCCGGCGATGAGCACAAGAAGCACCGGACCAAGGGAAAGGTGGT
GGTGGACTTCAGCAAGAAGGACGAGGACCAGAGCTACTATATCTCCAAGAACAACGTGATCGTGCGG
ATCGACAAGAACGCCGGCCCTAGAAGCTACCGGATGGGCCTGAACGAGCTGAAGTACCTCGTGCTGC
TGAGCCTGCAGGGGAAGGGCGACGATGCCATCGCCAAGCTGTACAGATACAGACAGCACGTGGAGA
ACATCCTGGATGTGGTGAAGGTGACCGATAAGGATAACCACGTGTTCCTGCCCCGCTTCGTGCTGGAG
CAGCACGGCATCGGCAGAAAGGCCTTCAAGCAGCGGATCGATGGACGGGTGAAGCACGTGCGGGGC
GTGTGGGAGAAGAAGAAGGCCGCCACCAATGAAATGACCCTGCACGAGAAGGCCAGAGACATCCTG
CAGTACGTGAACGAAAACTGCACCCGGTCCTTCAACCCTGGCGAATACAACAGACTGCTGGTGTGCC
TGGTGGGCAAGGACGTGGAGAACTTTCAGGCCGGCCTGAAGCGGCTGCAGCTGGCCGAAAGGATCG
ATGGCCGGGTGTACTCCATCTTCGCCCAGACCAGCACCATCAATGAGATGCACCAGGTGGTGTGCGAC
CAGATCCTGAACCGGCTGTGCAGAATCGGCGACCAGAAGCTGTACGATTACGTGGGACTGGGCAAGA
AGGACGAAATCGACTACAAGCAGAAGGTGGCCTGGTTCAAGGAGCACATCAGCATCCGGAGAGGAT
TCCTGAGAAAGAAGTTCTGGTACGATAGCAAGAAGGGATTCGCAAAGCTGGTGGAGGAACACCTGG
AGTCCGGCGGCGGCCAGCGCGACGTGGGCCTGGACAAGAAGTACTACCACATCGACGCCATCGGCA
GATTCGAGGGCGCCAACCCCGCCCTGTACGAGACCCTGGCCAGAGATCGGCTGTGCCTCATGATGGC
CCAGTACTTCCTGGGCAGCGTGAGAAAGGAACTGGGCAACAAGATTGTGTGGAGCAACGACAGCAT
CGAACTGCCTGTGGAAGGCTCTGTGGGAAATGAGAAGAGCATCGTGTTCTCCGTGTCTGACTACGGC
AAGCTGTACGTGCTGGACGATGCCGAATTCCTGGGCCGGATCTGCGAATACTTCATGCCCCACGAAAA
GGGCAAGATCCGGGCCCACACAGTGGCCGAAAAGGGCTTTAGAGCATACAACGACCTGCAGAAGAA
GTGCGTGGAGGCCGTGCTGGCTTTCGAAGAGAAGGTGGTGAAGGCCAAGAAGATGAGCGAGAAGG
AAGGCGCCCACTACATCGACTTCCGGGAGATCCTGGCCCAGACCATGTGCAAGGAGGCCGAAAAGA
CCGCAGTGAACAAGGTGAGACGCGCCTTCTTCCACCACCACCTGAAGTTCGTGATTGACGAGTTCGG
CCTGTTCAGCGACGTGATGAAGAAGTACGGCATCGAGAAGGAATGGAAGTTCCCTGTCAAG
Sequence encoding SV40 polyA signal,
SEQ ID NO: 21
AACTTGTTTATTGCAGCTTATAATGGTTACAAATAAAGCAATAGCATCACAAATTTCACAAATAAAGCA
TTTTTTTCACTGCATTCTAGTTGTGGTTTGTCCAAACTCATCAATGTATCTTATCATGTCTGGATC
U6 promoter,
SEQ ID NO: 22
GAGGGCCTATTTCCCATGATTCCTTCATATTTGCATATACGATACAAGGCTGTTAGAGAGATAATTGGAA
TTAATTTGACTGTAAACACAAAGATATTAGTACAAAATACGTGACGTAGAAAGTAATAATTTCTTGGGT
AGTTTGCAGTTTTAAAATTATGTTTTAAAATGGACTATCATATGCTTACCGTAACTTGAAAGTATTTCGA
TTTCTTGGCTTTATATATCTTGTGGAAAGGACGAAACACCG
hfCas13X scaffold sequence,
SEQ ID NO: 23
GCTGGAGCAGCCCCCGATTTGTGGGGTGATTACAGC
ITR-to-ITR (control),
SEQ ID NO: 24
CCTGCAGGCAGCTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAACTCCATCACTA
GGGGTTCCTGCGGCCATCGATGGCGCGCCGGATTCGACATTGATTATTGATAGGTCTTGAAAGGAGTG
GGAATTGGCTCCGGTGCCCGTCAGTGGGCAGAGCGCACATCGCCCACAGTCCCCGAGAAGTTGGGG
GGAGGGGTCGGCAATTGATCCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAGTAGTCGCCGTGAA
CGTTCTTTTTCGCAACGGGTTTGCCGCCAGAACACAGGGTTGGACCGGTGCCACCATGCCCAAGAAG
AAGCGGAAGGTGGCCCAGGTGAGCAAGCAGACCTCCAAGAAGAGGGAGCTGAGCATCGACGAGTA
CCAGGGCGCCCGGAAGTGGTGCTTCACCATTGCCTTCAACAAGGCCCTGGTGAACCGGGACAAGAA
CGACGGCCTGTTCGTGGAAAGCCTGCTGAGACACGAGAAGTACAGCAAGCACGACTGGTACGACGA
AGATACCCGGGCCCTGATCAAGTGCAGCACCCAGGCCGCCAACGCCAAGGCTGAAGCCCTGCGGAA
CTACTTCAGTCACTACCGGCATAGCCCTGGCTGCCTGACCTTCACCGCCGAGGACGAACTGCGGACC
ATCATGGAGAGAGCCTATGAGCGGGCCATCTTCGAGTGCAGAAGAAGAGAGACAGAGGTGATCATCG
AGTTTCCCAGCCTGTTCGAGGGCGACCGGATCACCACCGCCGGCGTGGTGTTTTTCGTGAGCTTTTTC
GTGGAAAGAAGAGTGCTGGATCGGCTGTATGGAGCCGTGTCCGGCCTGAAGAAGAATGAGGGACAG
TACAAGCTGACCCGGAAGGCCCTGAGCATGTACTGCCTGAAGGACAGCAGATTCACCAAGGCCTGGG
ATAAGCGGGTGCTGCTGTTCAGAGACATCCTGGCCCAGCTGGGAAGAATCCCCGCCGAGGCCTACGA
GTACTACCACGGCGAGCAGGGTGATAAGAAGAGAGCTAACGACAATGAGGGCACAAATCCCAAGCG
GCACAAGGACAAGTTCATCGAATTTGCACTGCACTACCTGGAAGCCCAGCACAGCGAGATCTGCTTC
GGCAGACGCCACATCGTGCGGGAAGAGGCCGGCGCCGGCGATGAGCACAAGAAGCACCGGACCAA
GGGAAAGGTGGTGGTGGACTTCAGCAAGAAGGACGAGGACCAGAGCTACTATATCTCCAAGAACAA
CGTGATCGTGCGGATCGACAAGAACGCCGGCCCTAGAAGCTACCGGATGGGCCTGAACGAGCTGAA
GTACCTCGTGCTGCTGAGCCTGCAGGGGAAGGGCGACGATGCCATCGCCAAGCTGTACAGATACAGA
CAGCACGTGGAGAACATCCTGGATGTGGTGAAGGTGACCGATAAGGATAACCACGTGTTCCTGCCCC
GCTTCGTGCTGGAGCAGCACGGCATCGGCAGAAAGGCCTTCAAGCAGCGGATCGATGGACGGGTGA
AGCACGTGCGGGGCGTGTGGGAGAAGAAGAAGGCCGCCACCAATGAAATGACCCTGCACGAGAAG
GCCAGAGACATCCTGCAGTACGTGAACGAAAACTGCACCCGGTCCTTCAACCCTGGCGAATACAACA
GACTGCTGGTGTGCCTGGTGGGCAAGGACGTGGAGAACTTTCAGGCCGGCCTGAAGCGGCTGCAGC
TGGCCGAAAGGATCGATGGCCGGGTGTACTCCATCTTCGCCCAGACCAGCACCATCAATGAGATGCAC
CAGGTGGTGTGCGACCAGATCCTGAACCGGCTGTGCAGAATCGGCGACCAGAAGCTGTACGATTACG
TGGGACTGGGCAAGAAGGACGAAATCGACTACAAGCAGAAGGTGGCCTGGTTCAAGGAGCACATCA
GCATCCGGAGAGGATTCCTGAGAAAGAAGTTCTGGTACGATAGCAAGAAGGGATTCGCAAAGCTGGT
GGAGGAACACCTGGAGTCCGGCGGCGGCCAGCGCGACGTGGGCCTGGACAAGAAGTACTACCACAT
CGACGCCATCGGCAGATTCGAGGGCGCCAACCCCGCCCTGTACGAGACCCTGGCCAGAGATCGGCTG
TGCCTCATGATGGCCCAGTACTTCCTGGGCAGCGTGAGAAAGGAACTGGGCAACAAGATTGTGTGGA
GCAACGACAGCATCGAACTGCCTGTGGAAGGCTCTGTGGGAAATGAGAAGAGCATCGTGTTCTCCGT
GTCTGACTACGGCAAGCTGTACGTGCTGGACGATGCCGAATTCCTGGGCCGGATCTGCGAATACTTCA
TGCCCCACGAAAAGGGCAAGATCCGGGCCCACACAGTGGCCGAAAAGGGCTTTAGAGCATACAACG
ACCTGCAGAAGAAGTGCGTGGAGGCCGTGCTGGCTTTCGAAGAGAAGGTGGTGAAGGCCAAGAAG
ATGAGCGAGAAGGAAGGCGCCCACTACATCGACTTCCGGGAGATCCTGGCCCAGACCATGTGCAAGG
AGGCCGAAAAGACCGCAGTGAACAAGGTGAGACGCGCCTTCTTCCACCACCACCTGAAGTTCGTGA
TTGACGAGTTCGGCCTGTTCAGCGACGTGATGAAGAAGTACGGCATCGAGAAGGAATGGAAGTTCCC
TGTCAAGCCCAAGAAGAAGCGGAAGGTGTAGAAGCTTAACTTGTTTATTGCAGCTTATAATGGTTACA
AATAAAGCAATAGCATCACAAATTTCACAAATAAAGCATTTTTTTCACTGCATTCTAGTTGTGGTTTGT
CCAAACTCATCAATGTATCTTATCATGTCTGGATCACTAGTGAGGGCCTATTTCCCATGATTCCTTCATAT
TTGCATATACGATACAAGGCTGTTAGAGAGATAATTGGAATTAATTTGACTGTAAACACAAAGATATTA
GTACAAAATACGTGACGTAGAAAGTAATAATTTCTTGGGTAGTTTGCAGTTTTAAAATTATGTTTTAAA
ATGGACTATCATATGCTTACCGTAACTTGAAAGTATTTCGATTTCTTGGCTTTATATATCTTGTGGAAAG
GACGAAACACCGGCTGGAGCAGCCCCCGATTTGTGGGGTGATTACAGCGTGCTGTAGGAAGCTCATC
TCTCCTATGTGGCTGGAGCAGCCCCCGATTTGTGGGGTGATTACAGCGGTACTCCTGGAAGATGTCCA
CCAGGGTCTGCTGGAGCAGCCCCCGATTTGTGGGGTGATTACAGCTTTTTTTACGCGTTAGTTATTAAT
AGTGCGTCGGCCGCAGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGGCCTCAGTGAGCGAG
CGAGCGCGCAGCTGCCTGCAGG
gRNA (S-g1-S-g2-S),
SEQ ID NO: 25
GCTGGAGCAGCCCCCGATTTGTGGGGTGATTACAGCGTGCTGTAGGAAGCTCATCTCTCCTATGTGGC
TGGAGCAGCCCCCGATTTGTGGGGTGATTACAGCGGTACTCCTGGAAGATGTCCACCAGGGTCTGCT
GGAGCAGCCCCCGATTTGTGGGGTGATTACAGC
SV40 NLS amino acid sequence,
SEQ ID NO: 26
PKKKRKV
napRNAn (hfCas13X) amino acid sequence,
SEQ ID NO: 27
MAQVSKQTSKKRELSIDEYQGARKWCFTIAFNKALVNRDKNDGLFVESLLRHEKYSKHDWYDEDTRAL
IKCSTQAANAKAEALRNYFSHYRHSPGCLTFTAEDELRTIMERAYERAIFECRRRETEVIIEFPSLFEGDRIT
TAGVVFFVSFFVERRVLDRLYGAVSGLKKNEGQYKLTRKALSMYCLKDSRFTKAWDKRVLLFRDILAQL
GRIPAEAYEYYHGEQGDKKRANDNEGTNPKRHKDKFIEFALHYLEAQHSEICFGRRHIVREEAGAGDEH
KKHRTKGKVVVDFSKKDEDQSYYISKNNVIVRIDKNAGPRSYRMGLNELKYLVLLSLQGKGDDAIAKLY
RYRQHVENILDVVKVTDKDNHVFLPRFVLEQHGIGRKAFKQRIDGRVKHVRGVWEKKKAATNEMTLHE
KARDILQYVNENCTRSFNPGEYNRLLVCLVGKDVENFQAGLKRLQLAERIDGRVYSIFAQTSTINEMHQV
VCDQILNRLCRIGDQKLYDYVGLGKKDEIDYKQKVAWFKEHISIRRGFLRKKFWYDSKKGFAKLVEEHLE
SGGGQRDVGLDKKYYHIDAIGRFEGANPALYETLARDRLCLMMAQYFLGSVRKELGNKIVWSNDSIELP
VEGSVGNEKSIVFSVSDYGKLYVLDDAEFLGRICEYFMPHEKGKIRAHTVAEKGFRAYNDLQKKCVEAVL
AFEEKVVKAKKMSEKEGAHYIDFREILAQTMCKEAEKTAVNKVRRAFFHHHLKFVIDEFGLFSDVMKKY
GIEKEWKFPVK
5′ ITR,
SEQ ID NO: 28
CCTGCAGGCAGCTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAACTCCATCACTA
GGGGTTCCT
3′ ITR,
SEQ ID NO: 29
AGGAACCCCTAGTGATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCGAGCGCGCAGCTG
CCTGCAGG
napRNAn (hfCas13Y) amino acid sequence with N-terminal Met removed,
SEQ ID NO: 32
NGIELKKEEAAFYENQAELNLKAIEDNIFDKERRKTLLNNPQILAKMENFIFNFRDVTKNAKGEIDCLLLK
LRELRNFYSHYVHKRDVRELSKGEKPILEKYYQFAIESTGSENVKLEIIENDAWLADAGVLFFLCIFLKKS
QANKLISGISGFKRNDATGQPRRNLFTYFSIREGYKVVPEMQKHFLLFSLVNHLSNQDDYIEKAHQPYDIG
EGLFFHRIASTFLNISGILRNMKFYTYQSKRIVEQRGELKREKDIFAWEEPFQGNSYFEINGHKGVIGEDEL
KELCYAFLIGNQDANKVEGRITQFLEKFRNANSVQQVKDDEMLKPEYFPANYFAESGVGRIKDRVLNRL
NKAIKSNKAKKGEIIAYDKMREVMAFINNSLPVDEKLKPKDYKRYLGMVRFWDREKDNIKREFETKEWS
KYLPSNFWTAKNLERVYGLAREKNAELFNKLKADVEKMDERELEKYQKINDAKDLANLRRLASDFGVK
WEEKDWDEYSGQIKKQITDSQKLTIMKQRITAGLKKKHGIENLNLRITIDINKSRKAVLNRIAIPRGFVKRH
ILGWQESEKVSKKIREAECEILLSKEYEELSKQFFQSKDYDKMTRINGLYEKNKLIALMAVYLMGQLRILF
KEHTKLADITKTTVDFKISDKVTVKIPFSNAPSLVYTMSSKAVDNIGNYGFSNKDKDKPILGKIDVIEKQR
MEFIKEVLGFEKYLFDDKIIDKSKFADTATHISFAEIVEELVEKGWDKDRLTKLKDARNKALHGEILTGTSF
DETKSLINELKK
napRNAn (hfCas13Y) coding sequence with 5′ start codon ATG removed,
SEQ ID NO: 33
AACGGAATCGAGCTGAAGAAGGAGGAAGCCGCCTTTTACTTCAATCAGGCCGAGCTGAACTTAAAG
GCCATCGAGGACAACATCTTTGACAAGGAGCGGAGAAAGACCCTGCTGAATAACCCTCAGATCCTGG
CCAAGATGGAGAACTTCATATTCAACTTCAGAGATGTGACAAAGAACGCCAAGGGCGAGATTGACTG
CCTGCTGCTGAAACTGAGAGAACTGAGAAACTTCTACAGCCACTACGTGCACAAGAGAGACGTGCG
GGAACTATCTAAAGGCGAGAAGCCTATCCTGGAGAAGTATTACCAGTTTGCCATCGAAAGCACCGGC
AGCGAAAACGTCAAGCTGGAAATCATCGAGAATGACGCTTGGCTGGCTGATGCCGGGGTGCTGTTTT
TCCTGTGCATCTTCCTGAAGAAGTCCCAAGCCAACAAGCTCATTAGCGGCATCAGCGGCTTCAAGAG
AAACGACGCTACCGGCCAGCCAAGAAGAAATCTGTTCACCTACTTCAGCATCAGAGAAGGCTACAAG
GTGGTGCCCGAGATGCAGAAGCACTTCCTGCTGTTCTCTCTGGTGAACCACCTGAGCAATCAGGACG
ACTATATCGAGAAGGCCCACCAACCCTACGACATCGGAGAGGGCCTGTTCTTCCACAGAATCGCCAG
CACCTTCCTGAACATCAGCGGCATCCTGAGAAACATGAAATTCTACACATACCAAAGCAAAAGACTG
GTCGAGCAGAGAGGCGAGCTGAAAAGGGAGAAGGACATTTTCGCCTGGGAAGAGCCTTTCCAGGGC
AACAGCTACTTCGAGATCAATGGCCACAAAGGAGTTATCGGCGAAGATGAACTGAAGGAACTGTGTT
ACGCCTTCCTGATCGGCAATCAAGACGCCAACAAGGTAGAAGGCAGAATCACACAGTTCCTGGAAAA
GTTCAGAAACGCCAACAGCGTGCAGCAGGTGAAGGACGATGAAATGCTGAAGCCTGAATACTTTCCT
GCAAACTACTTTGCCGAGAGCGGTGTGGGCAGAATCAAGGACCGGGTGCTGAACCGGCTGAACAAG
GCCATAAAGAGCAATAAAGCTAAGAAGGGCGAAATCATCGCCTACGATAAGATGCGGGAAGTGATGG
CCTTCATCAACAACAGCCTACCAGTGGATGAGAAGCTGAAGCCCAAGGATTACAAGCGGTACCTGGG
AATGGTCCGGTTCTGGGATAGAGAGAAGGATAACATCAAGCGCGAATTCGAGACAAAAGAGTGGTCC
AAATACCTGCCTAGCAATTTCTGGACCGCCAAGAACCTGGAGCGGGTGTACGGACTGGCCCGTGAGA
AAAACGCAGAGCTGTTCAACAAGCTGAAAGCTGACGTGGAAAAAATGGATGAGAGAGAACTGGAG
AAATACCAGAAGATCAACGACGCCAAAGACCTGGCTAACCTGCGGCGGCTGGCCTCTGACTTCGGCG
TGAAGTGGGAAGAGAAGGACTGGGACGAGTACTCTGGCCAGATCAAGAAGCAGATCACCGACAGCC
AGAAGCTGACCATCATGAAGCAGAGAATTACCGCAGGCCTGAAAAAAAAGCACGGCATCGAAAACC
TGAACCTGAGAATCACCATCGACATCAATAAGAGCAGAAAAGCCGTGCTGAATAGAATTGCCATCCCT
AGAGGCTTTGTGAAGAGACATATCCTCGGATGGCAGGAGTCTGAGAAGGTGTCCAAAAAAATCAGAG
AAGCCGAGTGCGAGATCCTGCTCTCCAAGGAGTATGAAGAGCTGAGCAAACAGTTCTTCCAGAGCAA
GGACTACGACAAAATGACCAGAATCAACGGCCTGTACGAGAAAAACAAGCTTATCGCCCTGATGGCC
GTGTACCTGATGGGCCAGCTGCGGATCCTGTTTAAGGAACACACAAAGCTGGCTGATATCACAAAAA
CCACAGTGGATTTCAAGATCAGCGACAAGGTGACAGTGAAGATTCCCTTCAGCAACGCTCCTAGCCT
GGTGTATACCATGAGCAGCAAGGCCGTGGACAACATCGGAAATTACGGCTTCAGCAACAAAGACAAG
GATAAGCCCATCCTGGGCAAGATCGATGTGATCGAGAAGCAGCGGATGGAGTTTATCAAGGAAGTCC
TGGGATTCGAGAAGTACCTGTTCGATGATAAGATCATCGACAAGAGCAAGTTCGCCGACACCGCCAC
CCACATCTCTTTCGCCGAGATCGTTGAAGAGCTGGTGGAAAAGGGATGGGACAAAGATCGGCTGACC
AAGCTGAAGGACGCTAGAAACAAGGCTCTGCATGGCGAAATCCTGACCGGCACCAGCTTCGACGAG
ACAAAGTCCCTGATCAACGAGCTGAAGAAG
napRNAn (hfCas13X) amino acid sequence with N-terminal Met removed,
SEQ ID NO: 34
AQVSKQTSKKRELSIDEYQGARKWCFTIAFNKALVNRDKNDGLFVESLLRHEKYSKHDWYDEDTRALIK
CSTQAANAKAEALRNYFSHYRHSPGCLTFTAEDELRTIMERAYERAIFECRRRETEVIIEFPSLFEGDRITTA
GVVFFVSFFVERRVLDRLYGAVSGLKKNEGQYKLTRKALSMYCLKDSRFTKAWDKRVLLFRDILAQLGRI
PAEAYEYYHGEQGDKKRANDNEGTNPKRHKDKFIEFALHYLEAQHSEICFGRRHIVREEAGAGDEHKKH
RTKGKVVVDFSKKDEDQSYYISKNNVIVRIDKNAGPRSYRMGLNELKYLVLLSLQGKGDDAIAKLYRYR
QHVENILDVVKVTDKDNHVFLPRFVLEQHGIGRKAFKQRIDGRVKHVRGVWEKKKAATNEMTLHEKAR
DILQYVNENCTRSFNPGEYNRLLVCLVGKDVENFQAGLKRLQLAERIDGRVYSIFAQTSTINEMHQVVCD
QILNRLCRIGDQKLYDYVGLGKKDEIDYKQKVAWFKEHISIRRGFLRKKFWYDSKKGFAKLVEEHLESGG
GQRDVGLDKKYYHIDAIGRFEGANPALYETLARDRLCLMMAQYFLGSVRKELGNKIVWSNDSIELPVEG
SVGNEKSIVFSVSDYGKLYVLDDAEFLGRICEYFMPHEKGKIRAHTVAEKGFRAYNDLQKKCVEAVLAFE
EKVVKAKKMSEKEGAHYIDFREILAQTMCKEAEKTAVNKVRRAFFHHHLKFVIDEFGLFSDVMKKYGIE
KEWKFPVK
napRNAn (hfCas13X) coding sequence with 5′ start codon ATG removed,
SEQ ID NO: 35
GCCCAGGTGAGCAAGCAGACCTCCAAGAAGAGGGAGCTGAGCATCGACGAGTACCAGGGCGCCCGG
AAGTGGTGCTTCACCATTGCCTTCAACAAGGCCCTGGTGAACCGGGACAAGAACGACGGCCTGTTCG
TGGAAAGCCTGCTGAGACACGAGAAGTACAGCAAGCACGACTGGTACGACGAAGATACCCGGGCCC
TGATCAAGTGCAGCACCCAGGCCGCCAACGCCAAGGCTGAAGCCCTGCGGAACTACTTCAGTCACTA
CCGGCATAGCCCTGGCTGCCTGACCTTCACCGCCGAGGACGAACTGCGGACCATCATGGAGAGAGCC
TATGAGCGGGCCATCTTCGAGTGCAGAAGAAGAGAGACAGAGGTGATCATCGAGTTTCCCAGCCTGT
TCGAGGGCGACCGGATCACCACCGCCGGCGTGGTGTTTTTCGTGAGCTTTTTCGTGGAAAGAAGAGT
GCTGGATCGGCTGTATGGAGCCGTGTCCGGCCTGAAGAAGAATGAGGGACAGTACAAGCTGACCCGG
AAGGCCCTGAGCATGTACTGCCTGAAGGACAGCAGATTCACCAAGGCCTGGGATAAGCGGGTGCTGC
TGTTCAGAGACATCCTGGCCCAGCTGGGAAGAATCCCCGCCGAGGCCTACGAGTACTACCACGGCGA
GCAGGGTGATAAGAAGAGAGCTAACGACAATGAGGGCACAAATCCCAAGCGGCACAAGGACAAGTT
CATCGAATTTGCACTGCACTACCTGGAAGCCCAGCACAGCGAGATCTGCTTCGGCAGACGCCACATC
GTGCGGGAAGAGGCCGGCGCCGGCGATGAGCACAAGAAGCACCGGACCAAGGGAAAGGTGGTGGT
GGACTTCAGCAAGAAGGACGAGGACCAGAGCTACTATATCTCCAAGAACAACGTGATCGTGCGGATC
GACAAGAACGCCGGCCCTAGAAGCTACCGGATGGGCCTGAACGAGCTGAAGTACCTCGTGCTGCTGA
GCCTGCAGGGGAAGGGCGACGATGCCATCGCCAAGCTGTACAGATACAGACAGCACGTGGAGAACA
TCCTGGATGTGGTGAAGGTGACCGATAAGGATAACCACGTGTTCCTGCCCCGCTTCGTGCTGGAGCA
GCACGGCATCGGCAGAAAGGCCTTCAAGCAGCGGATCGATGGACGGGTGAAGCACGTGCGGGGCGT
GTGGGAGAAGAAGAAGGCCGCCACCAATGAAATGACCCTGCACGAGAAGGCCAGAGACATCCTGCA
GTACGTGAACGAAAACTGCACCCGGTCCTTCAACCCTGGCGAATACAACAGACTGCTGGTGTGCCTG
GTGGGCAAGGACGTGGAGAACTTTCAGGCCGGCCTGAAGCGGCTGCAGCTGGCCGAAAGGATCGAT
GGCCGGGTGTACTCCATCTTCGCCCAGACCAGCACCATCAATGAGATGCACCAGGTGGTGTGCGACC
AGATCCTGAACCGGCTGTGCAGAATCGGCGACCAGAAGCTGTACGATTACGTGGGACTGGGCAAGAA
GGACGAAATCGACTACAAGCAGAAGGTGGCCTGGTTCAAGGAGCACATCAGCATCCGGAGAGGATT
CCTGAGAAAGAAGTTCTGGTACGATAGCAAGAAGGGATTCGCAAAGCTGGTGGAGGAACACCTGGA
GTCCGGCGGCGGCCAGCGCGACGTGGGCCTGGACAAGAAGTACTACCACATCGACGCCATCGGCAG
ATTCGAGGGCGCCAACCCCGCCCTGTACGAGACCCTGGCCAGAGATCGGCTGTGCCTCATGATGGCC
CAGTACTTCCTGGGCAGCGTGAGAAAGGAACTGGGCAACAAGATTGTGTGGAGCAACGACAGCATC
GAACTGCCTGTGGAAGGCTCTGTGGGAAATGAGAAGAGCATCGTGTTCTCCGTGTCTGACTACGGCA
AGCTGTACGTGCTGGACGATGCCGAATTCCTGGGCCGGATCTGCGAATACTTCATGCCCCACGAAAAG
GGCAAGATCCGGGCCCACACAGTGGCCGAAAAGGGCTTTAGAGCATACAACGACCTGCAGAAGAAG
TGCGTGGAGGCCGTGCTGGCTTTCGAAGAGAAGGTGGTGAAGGCCAAGAAGATGAGCGAGAAGGA
AGGCGCCCACTACATCGACTTCCGGGAGATCCTGGCCCAGACCATGTGCAAGGAGGCCGAAAAGACC
GCAGTGAACAAGGTGAGACGCGCCTTCTTCCACCACCACCTGAAGTTCGTGATTGACGAGTTCGGCC
TGTTCAGCGACGTGATGAAGAAGTACGGCATCGAGAAGGAATGGAAGTTCCCTGTCAAG

1. A system comprising:
(1) a guide nucleic acid comprising
c) a scaffold sequence capable of forming a complex with a Cas13 endonuclease, and
d) a guide sequence capable of hybridizing to a target sequence on a transcript of a VEGFA gene, thereby guiding the complex to the transcript, or a polynucleotide encoding the guide nucleic acid; and
(2) the Cas13 endonuclease or a polynucleotide encoding the Cas13 endonuclease;
wherein the Cas13 endonuclease comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO: 16 or 32, comprises substitutions D160A, D642A, Y666A, and Y677A relative to the wild type of the Cas13 endonuclease of SEQ ID NO: 16, and having substantially the same (e.g., at least about 80%, 90%, 95%, 99%, or more) guide sequence-specific RNA endonuclease activity as the Cas13 endonuclease of SEQ ID NO: 16;
wherein the guide sequence comprises (1) a sequence of SEQ ID NO: 1 or 2 or a 5′ or 3′ end truncation thereof with 1, 2, 3, 4, 5, or 6 nucleotides truncated at the 5′ or 3′ end; or (2) a sequence having a sequence identity of at least about 70%, 75%, 80%, 85%, 90%, 95%, or 100% to SEQ ID NO: 1 or 2 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 1 or 2.

2. The system of any preceding claim, wherein the scaffold sequence has substantially the same secondary structure as the secondary structure of the sequence of SEQ ID NO: 5; or wherein the scaffold sequence comprises (1) a sequence of SEQ ID NO: 5 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 5 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 5.

3. The system of any preceding claim, wherein the guide nucleic acid comprises, from 5′ to 3′, one said scaffold sequence, one said guide sequence, and one said scaffold sequence, wherein scaffold sequences are the same or different.

4. The system of any preceding claim, wherein the guide nucleic acid comprises, from 5′ to 3′, one said scaffold sequence, one said guide sequence, one said scaffold sequence, one said guide sequence, and one said scaffold sequence, wherein scaffold sequences are the same or different, and wherein guide sequences are the same or different.

5. The system of any preceding claim, wherein the guide nucleic acid comprises a sequence having a sequence identity of at least about 80% (e.g., at least about 80%, 85%, 90%, 91%, 92%, 93%, 94%, 95%, 96%, 97%, 98%, 99%, or 100%) to the sequence of any one of SEQ ID NOs: 17 and 30-31; or a sequence having at most 1, 2, 3, 4, 5, 6, 7, 8, 9, or 10 nucleotide differences, whether consecutive or not, compared to the sequence of any one of SEQ ID NOs: 17 and 30-31.

6. The system of any preceding claim, wherein the transcript of the VEGFA gene is a VEGFA pre-mRNA or a VEGFA mRNA.

7. The system of any preceding claim, wherein the VEGFA gene is human VEGFA gene.

8. A recombinant adeno-associated virus (rAAV) vector genome comprising:
(1) a first polynucleotide sequence comprising a sequence comprising or encoding the guide nucleic acid as defined in any preceding claim; and
(2) a second polynucleotide sequence comprising a sequence encoding the Cas13 endonuclease as defined in any preceding claim,
wherein the rAAV vector genome is adapted to be encapsulated into a rAAV particle.

9. The rAAV vector genome of any preceding claim, wherein the first polynucleotide sequence comprises a U6 promoter (e.g., SEQ ID NO: 4 or 22) operably linked to the sequence comprising or encoding the guide nucleic acid.

10. The rAAV vector genome of any preceding claim, wherein the second polynucleotide sequence comprises a Cbh promoter (e.g., SEQ ID NO: 6) operably linked to the sequence encoding the Cas13 endonuclease.

11. The rAAV vector genome of any preceding claim, wherein the second polynucleotide comprises a first sequence encoding a first NLS at the 5′ end of the sequence encoding the Cas13 endonuclease and/or a second sequence encoding a second NLS at the 3′ end of the sequence encoding the Cas13 endonuclease.

12. The rAAV vector genome of any preceding claim, wherein the first NLS and/or the second NLS is a SV40 NLS (e.g., SEQ ID NO: 26), a bpSV40 NLS (e.g., SEQ ID NO: 15), or a Nucleoplasmin NLS (npNLS).

13. The rAAV vector genome of any preceding claim, wherein the second polynucleotide sequence comprises a WPRE sequence downstream of the sequence encoding the Cas13 endonuclease; optionally wherein the WPRE sequence is selected from the group consisting of Woodchuck Hepatitis Virus (WHP) Posttranscriptional Regulatory Element (WPRE), WPRE3 (e.g., SEQ ID NO: 11), and a functional variant (e.g., a functional truncation) thereof.

14. The rAAV vector genome of any preceding claim, wherein the second polynucleotide sequence comprises a sequence encoding a bGH polyA signal (e.g., SEQ ID NO: 12) downstream of the sequence encoding the Cas13 endonuclease.

15. The rAAV vector genome of any preceding claim, wherein the second polynucleotide sequence comprises, from 5′ to 3′, the promoter of SEQ ID NO: 6, Kozak sequence of gccacc, an in-frame start codon ATG, the first sequence encoding the first NLS of SEQ ID NO: 15, the sequence encoding the Cas13 endonuclease of SEQ ID NO: 16 or 32, the second sequence encoding the second NLS of SEQ ID NO: 15, an in-frame stop codon, the WPRE sequence of SEQ ID NO: 11, and the sequence encoding the polyA signal of SEQ ID NO: 12.

16. The rAAV vector genome of any preceding claim, wherein the rAAV vector genome comprises, from 5′ to 3′, the first polynucleotide sequence and the second polynucleotide sequence; or comprises, from 5′ to 3′, the second polynucleotide sequence and the first polynucleotide sequence.

17. The rAAV vector genome of any preceding claim, wherein the rAAV vector genome comprises, from the 5′ to 3′,
(1) the 5′ITR of SEQ ID NO: 3;
(2) the promoter of SEQ ID NO: 4;
(3) a sequence encoding the scaffold sequence of SEQ ID NO: 5;
(4) a sequence encoding the guide sequence of SEQ ID NO: 1;
(5) a sequence encoding the scaffold sequence of SEQ ID NO: 5;
(6) a sequence encoding the guide sequence of SEQ ID NO: 2;
(7) a sequence encoding the scaffold sequence of SEQ ID NO: 5;
(8) the promoter of SEQ ID NO: 6;
(9) Kozak sequence of gccacc;
(10) in-frame start codon ATG;
(11) the first sequence of SEQ ID NO: 8 encoding the first NLS of SEQ ID NO: 15;
(12) the sequence of SEQ ID NO: 33 encoding the Cas13 endonuclease of SEQ ID NO: 32;
(13) the second sequence SEQ ID NO: 10 encoding the second NLS of SEQ ID NO: 15;
(14) an in-frame stop codon;
(15) the WPRE sequence of SEQ ID NO: 11;
(16) the sequence encoding a polyA signal of SEQ ID NO: 12; and
(17) the 3′ ITR of SEQ ID NO: 13;
wherein the rAAV vector genome comprises an optional linker between any two adjacent components of the components (1) to (17).

18. The rAAV vector genome of any preceding claim, wherein the rAAV vector genome comprises, consists essentially of, or consists of a polynucleotide having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 14.

19. The rAAV vector genome of any preceding claim, wherein the rAAV vector genome comprises, from the 5′ to 3′,
(1) the 5′ ITR of SEQ ID NO: 3;
(2) the promoter of SEQ ID NO: 4;
(3) a sequence encoding the scaffold sequence of SEQ ID NO: 5;
(4) a sequence encoding the guide sequence of SEQ ID NO: 1;
(5) a sequence encoding the scaffold sequence of SEQ ID NO: 5;
(6) a sequence encoding the guide sequence of SEQ ID NO: 2;
(7) a sequence encoding the scaffold sequence of SEQ ID NO: 5;
(8) the promoter of SEQ ID NO: 6;
(9) Kozak sequence of gccacc;
(10) in-frame start codon ATG;
(11) the first sequence of SEQ ID NO: 8 encoding the first NLS of SEQ ID NO: 15;
(12) the sequence of SEQ ID NO: 33 encoding the Cas13 endonuclease of SEQ ID NO: 32;
(13) the second sequence SEQ ID NO: 10 encoding the second NLS of SEQ ID NO: 15;
(14) an in-frame stop codon;
(15) the WPRE sequence of SEQ ID NO: 11;
(16) the sequence encoding a polyA signal of SEQ ID NO: 12; and
(17) the 3′ ITR of SEQ ID NO: 13;
wherein the rAAV vector genome comprises a sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the sequence of SEQ ID NO: 14;
wherein the rAAV vector genome does not contain any nucleotide difference from the sequence of SEQ ID NO: 14 in any one of the components (1) to (17);
wherein the rAAV vector genome contains one or more nucleotide difference or does not contain any nucleotide difference from the sequence of SEQ ID NO: 14 between any two adjacent components of the components (1) to (17).

20. The rAAV vector genome of any preceding claim, wherein the rAAV vector genome comprises, consists essentially of, or consists of the sequence of SEQ ID NO: 14.

21. A recombinant AAV (rAAV) particle comprising the rAAV vector genome of any preceding claim.

22. The rAAV particle of any preceding claim, wherein the rAAV particle comprising a capsid with a serotype of AAV8.

23. The rAAV particle of any preceding claim, wherein the AAV8 is wild type AAV8 or a functional variant thereof.

24. A method for production of a rAAV particle, comprising culturing in a host cell a transgene plasmid comprising the rAAV vector genome of any preceding claim, thereby encapsulating the rAAV vector genome into a capsid with a serotype of AAV8.

25. A cell comprising a transgene plasmid comprising the rAAV vector genome of any preceding claim.

26. Use of the cell comprising a transgene plasmid comprising the rAAV vector genome of any preceding claim for the production of rAAV particles.

27. A pharmaceutical composition comprising (1) the system of any preceding claim or the rAAV particle of any preceding claim and (2) a pharmaceutically acceptable excipient.

28. A method of downregulating expression of a VEGFA gene, comprising contacting the transcript of the VEGFA gene with the system of any preceding claim, the rAAV particle of any preceding claim, or the pharmaceutical composition of any preceding claim.

29. A cell or a progeny thereof comprising the system of any preceding claim, or the rAAV particle of any preceding claim.

30. A cell or a progeny thereof comprising a VEGFA transcript modified by the system of any preceding claim, the rAAV particle of any preceding claim, or the method of any preceding claim.

31. A method for preventing, diagnosing, and/or treating a VEGFA associated disease in a subject in need thereof, comprising administering to the subject the system of any preceding claim, the rAAV particle of any preceding claim, or the pharmaceutical composition of any preceding claim, wherein the Cas13 endonuclease modifies the transcript of VEGFA gene, leading to downregulated expression of VEGFA gene, thereby treating the VEGFA associated disease.

32. The method of any preceding claim, wherein the VEGFA associated disease is neovascular age-related macular degeneration (nAMD), Diabetic Macular Edema (DME), or a cancer.

33. The method of any preceding claim, wherein the subject is not responsive to anti-VEGF therapy, such as, anti-VEGF antibody.
